(19) United States
(12) Patent Application Publication
(10) Pub. No.: US 2025/0362305 A1
(43) Pub. Date: Nov. 27, 2025

(54) METHODS FOR DETERMINING ONE OR MORE CRITICAL QUALITY ATTRIBUTES OF CO-FORMULATED ANTIBODIES

(71) Applicant: MERCK SHARP & DOHME LLC, Rahway, NJ (US)

(72) Inventors: Chengdong XU, East Brunswick, NJ (US); Christoper A. STRULSON, Scotch Plains, NJ (US); Nicholas A. PIERSON, Cranford, NJ (US); Brent A. KOCHERT, Montclair, NJ (US)

(73) Assignee: MERCK SHARP & DOHME LLC, Rahway, NJ (US)

(21) Appl. No.: 19/100,667

(22) PCT Filed: Aug. 2, 2023
(86) PCT No.: PCT/US2023/029257

§ 371 Date: Feb. 3, 2025

Related U.S. Application Data
(60) Provisional application No. 63/395,201, filed on Aug. 4, 2022.

(51) Int. Cl. G01N 33/68

(52) U.S. Cl. CPC G01N 33/6848; G01N 33/6854; G01N 2333/70503; G01N 2333/70521; G01N 2333/95

(57) ABSTRACT

The present disclosure relates to methods for determining one or more critical quality attributes of co-formulated antibodies. The present disclosure also relates to methods comprising preparing a sample of a co-formulation comprising two or more different types of antibodies; and performing an analytical method on the sample to measures the critical quality attribute of each of the two or more different types of antibodies simultaneously.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Dec. 8, 2022, is named 25567-WO-PCT_SL. XML and is 113,235 bytes in size.

### BACKGROUND OF THE INVENTION

The strategy of using two or more antibodies as combination therapy has been explored in recent years. Among the combination therapy strategies, co-formulated drug products offer unique advantages of reducing dosing complexity, adverse events, or health care costs, as well as directly improving patient convenience and compliance. In 2020, the first co-formulated antibody drug product was approved by U.S. Food and Drug Administration for breast cancer therapy.

Regulatory agencies have recommended a Quality by Design (QbD) approach for the manufacturing of therapeutic molecules. A QbD strategy requires understanding at the molecular level the attributes that are important for safety and efficacy, and insuring that the desired quality of the protein drug product is met at the end of the manufacturing process.

Therapeutic proteins are subject to various post-translational modifications (PTMs). Certain PTMs may affect bioactivity, stability, or the pharmacokinetics and pharmacodynamics profile. Identifying, monitoring and controlling these PTMs are usually key elements of the QbD approach.

### SUMMARY OF THE INVENTION

The present disclosure extends an insight that co-formulated drug products (e.g., drug products comprising two or more different antibodies or antigen binding fragments thereof) can create analytical challenges, including analysis of PTMs (e.g., oxidation, isomerization, deamidation, disulfide bond modification, glycosylation, etc.). For example, when two antibodies have similar physicochemical properties and/or large concentration disparity, analysis can be difficult.

The present disclosure is based, in part, on an insight that a co-formulation of two or more different antibodies or antigen binding fragments thereof may be analyzed differently from a mono-formulation comprising one type of antibodies or antigen binding fragments thereof. Chromatography-based methods, such as hydrophobic interaction chromatography (HIC) and reverse phase liquid chromatography (RPLC), have been applied to analyze protein PTMs (e.g., oxidation) with limited success. Low selectivity and sensitivity of ETC often results in peak co-elution and insufficient accuracy. The present disclosure recognizes that this challenge can be exacerbated when two co-formulated antibodies have similar properties, e.g., hydrophobicity. Similarly, RPLC has been used for analysis of PTMs (e.g., total oxidation) of intact antibody. The present disclosure also appreciates that, while RPLC can provide improved selectivity compared to HIC, the resolution between antibodies with PTMs (e.g., oxidation) and native antibodies is often not sufficient to allow reliable quantitation of the PTMs for coformulation.

In one aspect, provided are methods for determining a critical quality attribute of a co-formulation, that include a step of preparing a sample of a co-formulation, and performing an analytical method on the sample. In some embodiments, the co-formulation comprises two or more different types of antibodies or antigen binding fragments thereof. In some embodiments, the analytical method measures a critical quality attribute of each of the two or more different types of antibodies or antigen binding fragments thereof simultaneously.

In some embodiments, wherein the co-formulation comprises an anti-PD-1 antibody or antigen binding fragment thereof and an anti-TIGIT antibody or antigen binding fragment thereof.

In some embodiments, the anti-PD-1 antibody or antigen binding fragment thereof is a monoclonal antibody. In some embodiments, the anti-PD-1 antibody or antigen binding fragment thereof is a humanized antibody. In some embodiments, the anti-PD-1 antibody or antigen binding fragment thereof is a human antibody.

In some embodiments, the anti-TIGIT antibody or antigen binding fragment thereof is a monoclonal antibody. In some embodiments, the anti-TIGIT antibody or antigen binding fragment thereof is a humanized antibody. In some embodiments, the anti-TIGIT antibody or antigen binding fragment thereof is a human antibody.

In some embodiments, the anti-PD-1 antibody or antigen binding fragment thereof comprises a light chain variable region (V_{L}) complementarity determining region (CDR) 1, a V_{L} CDR2, and a V_{L} CDR3 comprising amino acid sequences as set forth in SEQ ID NOs:1, 2, and 3, respectively, and a heavy chain variable region (V_{H}) CDR1, a V_{H} CDR2, and a V_{H} CDR3 comprising amino acid sequences as set forth in SEQ ID NOS:6, 7, and 8, respectively. In some embodiments, the anti-PD-1 antibody or antigen binding fragment thereof comprises a V_{L} region comprising an amino acid sequence as set forth in SEQ ID NO:4, and a V_{H} region comprising an amino acid sequence as set forth in SEQ LD NO:9. In some embodiments, the anti-PD-1 antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10.

In some embodiments, the anti-PD-1 antibody or antigen binding fragment thereof comprises a V_{L} CDR1, a V_{L} CDR2, and a V_{L} CDR3 comprising amino acid sequences as set forth in SEQ ID NOs:31, 32, and 33, respectively, and a V_{H} CDR1, a V_{H} CDR2, and a V_{H} CDR3 comprising amino acid sequences as set forth in SEQ ID NOs:36, 37, and 38, respectively. In some embodiments, the anti-PD-1 antibody or antigen binding fragment thereof comprises a V_{L} region comprising an amino acid sequence as set forth in SEQ ID NO:34, and a V_{H} region comprising an amino acid sequence as set forth in SEQ ID NO:39. In some embodiments, the anti-PD-1 antibody or antigen binding fragment thereof comprises a light chain comprising or consisting of an amino acid sequence as set forth in SEQ ID NO:35 and a heavy chain comprising or consisting of an amino acid sequence as set forth in SEQ ID NO:40.

In some embodiments, the anti-PD-1 antibody or antigen binding fragment thereof is pembrolizumab.

In some embodiments, the anti-PD-1 antibody or antigen binding fragment thereof is nivolumab.

In some embodiments, the anti-PD-1 antibody or antigen binding fragment thereof is cemiplimab.

In some embodiments, the anti-TIGIT antibody or antigen binding fragment thereof comprises a V_{L} CDR1, a V_{L} CDR2, and a V_{L} CDR3 comprising amino acid sequences as set forth in SEQ ID NOs:21, 22, and 23, respectively, and a V_{H} CDR1, a V_{H} CDR2, and a V_{H} CDR3 comprising amino acid sequences as set forth in SEQ ID NOs:26, 27, and 28, respectively. In some embodiments, the anti-TIGIT antibody or antigen binding fragment thereof comprises a V_{L} region comprising an amino acid sequence as set forth in SEQ ID NO:24, and a V_{H} region comprising an amino acid sequence as set forth in SEQ ID NO:29. In some embodiments, the anti-TIGIT antibody or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.

In some embodiments, the anti-PD-1 antibody or antigen binding fragment thereof comprises a V_{L} CDR1, a V_{L} CDR2, and a V_{L} CDR3 comprising amino acid sequences as set forth in SEQ ID NOs:1, 2, and 3, respectively, and a V_{H} CDR1, a V_{H} CDR2, and a V_{H} CDR3 comprising amino acid sequences as set forth in SEQ ID NOs:6, 7, and 8, respectively; and the anti-TIGIT antibody or antigen binding fragment thereof comprises a V_{L} CDR1, a V_{L} CDR2, and a V_{L} CDR3 comprising amino acid sequences as set forth in SEQ ID NOs:21, 22, and 23, respectively, and a V_{H} CDR1, a V_{H} CDR2, and a V_{H} CDR3 comprising amino acid sequences as set forth in SEQ ID NOs 26, 27, and 28, respectively.

In some embodiments, the anti-PD-1 antibody or antigen binding fragment thereof comprises a V_{L} region comprising an amino acid sequence as set forth in SEQ ID NO:4, and a V_{H} region comprising an amino acid sequence as set forth in SEQ ID NO:9; and the anti-TIGIT antibody or antigen binding fragment thereof comprises a V_{L} region comprising an amino acid sequence as set forth in SEQ ID NO:24, and a V_{H} region comprising an amino acid sequence as set forth in SEQ ID NO:29.

In some embodiments, the anti-PD-1 antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10; and the anti-TIGIT antibody or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.

In some embodiments, a ratio of the anti-PD-1 antibody or antigen binding fragment thereof to the anti-TIGIT antibody or antigen binding fragment thereof is within a range of about 2:1 to 1:2.

In some embodiments, the analytical method excludes hydrophobic interaction chromatography (HIC) and reverse phase liquid chromatography (RPLC). In some embodiments, the analytical method is a liquid chromatography-mass spectrometry (LC-MS) technique. In some embodiments, the LC-MS technique comprises the use of a quadruple Dalton mass detector.

In some embodiments, the step of preparing the sample comprises digesting the two or more different types of antibodies by mixing a protease with the sample. In some embodiments, the protease is selected from the group consisting of Arg-C, Asp-N, chymotrypsin, elastase, endo H, Glu-C, IdeS Protease, IdeZ Protease, Lys-C, Lys-N, pepsin, PNGase F, rAsp-N, rLys-C, thermolysin, trypsin, and combinations thereof. In some embodiments, the protease comprises Lys-C. In some embodiments, a concentration of Lys-C is within a range of about 0.005 to 0.01 g/L. In some embodiments, the protease is mixed with the sample for about 60 mins to 70 mins at a temperature within a range of about 35° C. to 40° C.

In some embodiments, the step of digesting comprises mixing a reducing agent solution with the sample. In some embodiments, the reducing agent solution comprises dithiothreitol and/or tris(2-carboxyethyl)phosphine. In some embodiments, the reducing agent solution is mixed with the sample for about 25 mins to 35 mins at a temperature within a range of about 35° C. to 40° C.

In some embodiments, the step of digesting comprises mixing an alkylating agent with the sample. In some embodiments, the alkylating agent comprises iodoacetamide. In some embodiments, the alkylating agent is mixed with the sample for about 25 mins to 35 mins at a temperature within a range of about 35° C. to 40° C.

In some embodiments, the analytical method comprises (i) applying the co-formulation to a chromatography material; and (ii) eluting with a solution comprising a mobile phase A and a mobile phase B. In some embodiments, the mobile phase A comprises formic acid or trifluoracetic acid. In some embodiments, the mobile phase B comprises formic acid or trifluoracetic acid. In some embodiments, the mobile phase A comprises acetic acid in water. In some embodiments, the mobile phase B comprises acetic acid in acetonitrile. In some embodiments, an initial ratio of the mobile phase B to the mobile phase A is within a range of about 10% to 20% with a flow rate within a range of about 0.1 mL/min to 1 mL/min.

In some embodiments, the chromatography is conducted at a temperature within a range of a range of about 60° C. to 100° C.

In some embodiments, the chromatography is ultra-performance liquid chromatography (UPLC).

In some embodiments, the elution is a gradient elution.

In some embodiments, an electrospray ionization probe of the quadruple Dalton mass detector is at a temperature within a range of about 350° C. to 450° C.

In some embodiments, a capillary voltage of the quadruple Dalton mass detector is at a temperature within a range of about 1 to 2 kV.

In some embodiments, the co-formulation comprises an anti-PD-1 antibody or antigen binding fragment thereof and an anti-LAG3 antibody or antigen binding fragment thereof.

In some embodiments, the anti-PD-1 antibody or antigen binding fragment thereof is a monoclonal antibody. In some embodiments, the anti-PD-1 antibody or antigen binding fragment thereof is a humanized antibody. In some embodiments, the anti-PD-1 antibody or antigen binding fragment thereof is a human antibody.

In some embodiments, the anti-LAG3 antibody or antigen binding fragment thereof is a monoclonal antibody. In some embodiments, the anti-LAG3 antibody or antigen binding fragment thereof is a humanized antibody. In some embodiments, the anti-LAG3 antibody or antigen binding fragment thereof is a human antibody.

In some embodiments, the anti-PD-1 antibody or antigen binding fragment thereof comprises a light chain variable region (V_{L}) complementarity determining region (CDR) 1, a V_{L} CDR2, and a V_{L} CDR3 comprising amino acid sequences as set forth in SEQ ID NOs:1, 2, and 3, respectively, and a heavy chain variable region (V_{H}) CDR1, a V_{H} CDR2, and a V_{H} CDR3 comprising amino acid sequences as set forth in SEQ ID NOS:6, 7, and 8, respectively. In some embodiments, the anti-PD-1 antibody or antigen binding fragment thereof comprises a V_{L} region comprising an amino acid sequence as set forth in SEQ ID NO:4, and a V_{H} region comprising an amino acid sequence as set forth in SEQ ID NO:9. In some embodiments, the anti-PD-1 antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10.

In some embodiments, the anti-PD-1 antibody or antigen binding fragment thereof comprises a V_{L} CDR1, a V_{L} CDR2, and a V_{L} CDR3 comprising amino acid sequences as set forth in SEQ ID NOs:31, 32, and 33, respectively, and a V_{H} CDR1, a V_{H} CDR2, and a V_{H} CDR3 comprising amino acid sequences as set forth in SEQ ID NOs:36, 37, and 38, respectively. In some embodiments, the anti-PD-1 antibody or antigen binding fragment thereof comprises a V_{L} region comprising an amino acid sequence as set forth in SEQ ID NO:34, and a V_{H} region comprising an amino acid sequence as set forth in SEQ ID NO:39. In some embodiments, the anti-PD-1 antibody or antigen binding fragment thereof comprises a light chain comprising or consisting of an amino acid sequence as set forth in SEQ ID NO:35 and a heavy chain comprising or consisting of an amino acid sequence as set forth in SEQ ID NO:40.

In some embodiments, the anti-PD-1 antibody or antigen binding fragment thereof is pembrolizumab.

In some embodiments, the anti-PD-1 antibody or antigen binding fragment thereof is nivolumab.

In some embodiments, the anti-PD-1 antibody or antigen binding fragment thereof is cemiplimab.

In some embodiments, the anti-LAG3 antibody or antigen binding fragment thereof comprises a V_{L} CDR1, a V_{L} CDR2, and a V_{L} CDR3 comprising amino acid sequences as set forth in SEQ ID NOs:11, 12, and 13, respectively, and a V_{H} CDR1, a V_{H} CDR2, and a V_{H} CDR3 comprising amino acid sequences as set forth in SEQ ID NOS:16, 17, and 18, respectively. In some embodiments, the anti-LAG3 antibody or antigen binding fragment thereof comprises a V_{L} region comprising an amino acid sequence as set forth in SEQ ID NO:14, and a V_{H} region comprising an amino acid sequence as set forth in SEQ ID NO:19. In some embodiments, the anti-LAG3 antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:20.

In some embodiments, the anti-PD-1 antibody or antigen binding fragment thereof comprises a V_{L} CDR1, a V_{L} CDR2, and a V_{L} CDR3 comprising amino acid sequences as set forth in SEQ ID NOs:1, 2, and 3, respectively, and a V_{H} CDR1, a V_{H} CDR2, and a V_{H} CDR3 comprising amino acid sequences as set forth in SEQ ID NOs:6, 7, and 8, respectively; and the anti-LAG3 antibody or antigen binding fragment thereof comprises a V_{L} CDR1, a V_{L} CDR2, and a V_{L} CDR3 comprising amino acid sequences as set forth in SEQ ID NOs:11, 12, and 13, respectively, and a V_{H} CDR1, a V_{H} CDR2, and a V_{H} CDR3 comprising amino acid sequences as set forth in SEQ ID NOs:16, 17, and 18, respectively.

In some embodiments, the anti-PD-1 antibody or antigen binding fragment thereof comprises a V_{L} region comprising an amino acid sequence as set forth in SEQ ID NO:4, and a V_{H} region comprising an amino acid sequence as set forth in SEQ ID NO:9; and the anti-LAG3 antibody or antigen binding fragment thereof comprises a V_{L} region comprising an amino acid sequence as set forth in SEQ ID NO:14, and a V_{H} region comprising an amino acid sequence as set forth in SEQ ID NO:19.

In some embodiments, the anti-PD-1 antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10; and the anti-LAG3 antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:20.

In some embodiments, a ratio of the anti-PD-1 antibody or antigen binding fragment thereof to the anti-LAG3 antibody or antigen binding fragment thereof is within a range of about 1:1 to 1:10.

In some embodiments, the analytical method excludes hydrophobic interaction chromatography (HIC) and reverse phase liquid chromatography (RPLC). In some embodiments, the analytical method is a liquid chromatography-mass spectrometry (LC-MS) technique. In some embodiments, the LC-MS technique comprises the use of a quadruple Dalton mass detector.

In some embodiments, the step of preparing the sample comprises digesting the two or more different types of antibodies by mixing a protease with the sample. In some embodiments, the protease is selected from the group consisting of Arg-C, Asp-N, chymotrypsin, elastase, endo H, Glu-C, IdeS Protease, IdeZ Protease, Lys-C, Lys-N, pepsin, PNGase F, rAsp-N, rLys-C, thermolysin, trypsin, and combinations thereof. In some embodiments, the protease comprises trypsin. In some embodiments, a concentration of trypsin in the sample is within a range from about 0.008 to 0.02 g/L. In some embodiments, the protease is mixed with the sample for about 25 mins to 35 mins at a temperature within a range of about 35° C. to 40° C.

In some embodiments, the step of digesting comprises mixing a reducing agent solution with the sample. In some embodiments, the reducing agent solution comprises dithiothreitol. In some embodiments, the reducing agent solution is mixed with the sample for about 5 mins to 15 mins at a temperature within a range of about 70° C. to 90° C.

In some embodiments, the analytical method comprises (i) applying the co-formulation to a chromatography material, and (ii) eluting with a solution comprising a mobile phase A and a mobile phase B. In some embodiments, the mobile phase A comprises formic acid or trifluoracetic acid. In some embodiments, the mobile phase B comprises formic acid or trifluoracetic acid. In some embodiments, the mobile phase A comprises acetic acid in water. In some embodiments, the mobile phase B comprises acetic acid in acetonitrile. In some embodiments, an initial ratio of the mobile phase B to the mobile phase A is within a range of about 10% to 30% with a flow rate within a range of about 0.1 mL/min to 1 mL/min.

In some embodiments, the chromatography is conducted at a temperature within a range of about 45° C. to 85° C.

In some embodiments, the chromatography is ultra-performance liquid chromatography (UPLC).

In some embodiments, the elution is a gradient elution.

In some embodiments, an electrospray ionization probe of the quadruple Dalton mass detector is at a temperature within a range of about 500° C. to 650° C.

In some embodiments, a capillary voltage of the quadruple Dalton mass detector is at a temperature within a range of about 1 to 2 kV.

In some embodiments, the co-formulation comprises an anti-PD-1 antibody or antigen binding fragment thereof and an anti-ILT4 antibody or antigen binding fragment thereof.

In some embodiments, the anti-PD-1 antibody or antigen binding fragment thereof is a monoclonal antibody. In some embodiments, the anti-PD-1 antibody or antigen binding fragment thereof is a humanized antibody. In some embodiments, the anti-PD-1 antibody or antigen binding fragment thereof is a human antibody.

In some embodiments, the anti-ILT4 antibody or antigen binding fragment thereof is a monoclonal antibody. In some embodiments, the anti-ILT4 antibody or antigen binding fragment thereof is a humanized antibody. In some embodiments, the anti-ILT4 antibody or antigen binding fragment thereof is a human antibody.

In some embodiments, the anti-PD-1 antibody or antigen binding fragment thereof comprises a light chain variable region (V_{L}) complementarity determining region (CDR) 1, a V_{L} CDR2, and a V_{L} CDR3 comprising amino acid sequences as set forth in SEQ ID NOs:1, 2, and 3, respectively, and a heavy chain variable region (V_{H}) CDR1, a V_{H} CDR2, and a V_{H} CDR3 comprising amino acid sequences as set forth in SEQ ID NOS:6, 7, and 8, respectively. In some embodiments, the anti-PD-1 antibody or antigen binding fragment thereof comprises a V_{L} region comprising an amino acid sequence as set forth in SEQ ID NO:4, and a V_{H} region comprising an amino acid sequence as set forth in SEQ ID NO:9. In some embodiments, the anti-PD-1 antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10.

In some embodiments, the anti-PD-1 antibody or antigen binding fragment thereof comprises a V_{L} CDR1, a V_{L} CDR2, and a V_{L} CDR3 comprising amino acid sequences as set forth in SEQ ID NOs:31, 32, and 33, respectively, and a V_{H} CDR1, a V_{H} CDR2, and a V_{H} CDR3 comprising amino acid sequences as set forth in SEQ ID NOs:36, 37, and 38, respectively. In some embodiments, the anti-PD-1 antibody or antigen binding fragment thereof comprises a V_{L} region comprising an amino acid sequence as set forth in SEQ ID NO:34, and a V_{H} region comprising an amino acid sequence as set forth in SEQ ID NO:39. In some embodiments, the anti-PD-1 antibody or antigen binding fragment thereof comprises a light chain comprising or consisting of an amino acid sequence as set forth in SEQ ID NO:35 and a heavy chain comprising or consisting of an amino acid sequence as set forth in SEQ ID NO:40.

In some embodiments, the anti-PD-1 antibody or antigen binding fragment thereof is pembrolizumab.

In some embodiments, the anti-PD-1 antibody or antigen binding fragment thereof is nivolumab.

In some embodiments, the anti-PD-1 antibody or antigen binding fragment thereof is cemiplimab.

In some embodiments, the anti-ILT4 antibody or antigen binding fragment thereof comprises a V_{L} CDR1, a V_{L} CDR2, and a V_{L} CDR3 comprising amino acid sequences as set forth in SEQ ID NOs:105, 106, and 107, respectively, and a V_{H} CDR1, a V_{H} CDR2, and a V_{H} CDR3 comprising amino acid sequences as set forth in SEQ ID NOs:110, 111, and 112, respectively. In some embodiments, the anti-ILT4 antibody or antigen binding fragment thereof comprises a V_{L} region comprising an amino acid sequence as set forth in SEQ ID NO:108, and a V_{H} region comprising an amino acid sequence as set forth in SEQ ID NO:113. In some embodiments, the anti-ILT4 antibody or antigen binding fragment thereof comprises a light chain comprising or consisting of an amino acid sequence as set forth in SEQ ID NO:109 and a heavy chain comprising or consisting of an amino acid sequence as set forth in SEQ ID NO:114.

In some embodiments, the anti-PD-1 antibody or antigen binding fragment thereof comprises a V_{L} CDR), a V_{L} CDR2, and a V_{L} CDR3 comprising amino acid sequences as set forth in SEQ ID NOs:1, 2, and 3, respectively, and a V_{H} CDR1, a V_{H} CDR2, and a V_{H} CDR3 comprising amino acid sequences as set forth in SEQ ID NOs:6, 7, and 8, respectively; and the anti-ILT4 antibody or antigen binding fragment thereof comprises a V_{L} CDR1, a V_{L} CDR2, and a V_{L} CDR3 comprising amino acid sequences as set forth in SEQ ID NOs:105, 106, and 107, respectively, and a V_{H} CDR1, a V_{H} CDR2, and a V_{H} CDR3 comprising amino acid sequences as set forth in SEQ ID NOs:110, 111, and 112, respectively.

In some embodiments, the anti-PD-1 antibody or antigen binding fragment thereof comprises a V_{L} region comprising an amino acid sequence as set forth in SEQ ID NO:4, and a V_{H} region comprising an amino acid sequence as set forth in SEQ ID NO:9; and the anti-ILT4 antibody or antigen binding fragment thereof comprises a V_{L} region comprising an amino acid sequence as set forth in SEQ ID NO:108, and a V_{H} region comprising an amino acid sequence as set forth in SEQ ID NO:113.

In some embodiments, the anti-PD-1 antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10; and the anti-ILT4 antibody or antigen binding fragment thereof comprises a light chain comprising or consisting of an amino acid sequence as set forth in SEQ ID NO:109 and a heavy chain comprising or consisting of an amino acid sequence as set forth in SEQ ID NO: 114.

In some embodiments, a ratio of the anti-PD-1 antibody or antigen binding fragment thereof to the anti-ILT4 antibody or antigen binding fragment thereof is within a range of about 2:1 to 1:2.

In some embodiments, the analytical method excludes hydrophobic interaction chromatography (HIC) and reverse phase liquid chromatography (RPLC). In some embodiments, the analytical method is a liquid chromatography-mass spectrometry (LC-MS) technique. In some embodiments, the LC-MS technique comprises the use of a quadruple Dalton mass detector.

In some embodiments, the step of preparing the sample comprises digesting the two or more different types of antibodies by mixing a protease with the sample. In some embodiments, the protease is selected from the group consisting of Arg-C. Asp-N, chymotrypsin, elastase, endo H, GIu-C, IdeS Protease, IdeZ Protease, Lys-C, Lys-N, pepsin, PNGase F, rAsp-N, rLys-C, thermolysin, trypsin, and combinations thereof. In some embodiments, the protease comprises Lys-C. In some embodiments, a concentration of Lys-C in the sample is within a range from about 0.005 to 0.01 g/L In some embodiments, the protease is mixed with the sample for about 60 mins to 70 mins at a temperature within a range of about 35° C. to 40° C.

In some embodiments, the step of digesting comprises mixing a reducing agent solution with the sample. In some embodiments, the reducing agent solution comprises dithiothreitol. In some embodiments, the reducing agent solution is mixed with the sample for about 25 mins to 35 mins at a temperature within a range of about 35° C. to 40° C.

In some embodiments, the step of digesting comprises mixing an alkylating agent with the sample. In some embodiments, the alkylating agent comprises iodoacetamide. In some embodiments, the alkylating agent is mixed with the sample for about 15 mins to 45 mins.

In some embodiments, the analytical method comprises (i) applying the co-formulation to a chromatography material; and (ii) eluting with a solution comprising a mobile phase A and a mobile phase B. In some embodiments, the mobile phase A comprises formic acid or trifluoracetic acid. In some embodiments, the mobile phase B comprises formic acid or trifluoracetic acid. In some embodiments, the mobile phase A comprises acetic acid in water. In some embodiments, the mobile phase B comprises acetic acid in acetonitrile. In some embodiments, an initial ratio of the mobile phase B to the mobile phase A is within a range of about 10% to 20% with a flow rate within a range of about 0.1 mL/min to 1 mL/min.

In some embodiments, the chromatography is conducted at a temperature within a range of about 40° C. to 80° C.

In some embodiments, the chromatography is ultra-performance liquid chromatography (UPLC).

In some embodiments, the elution is a gradient elution.

In some embodiments, an electrospray ionization probe of the quadruple Dalton mass detector is at a temperature within a range of about 350° C. to 450° C.

In some embodiments, a capillary voltage of the quadruple Dalton mass detector is at a temperature within a range of about 1 to 2 kV.

In some embodiments, the critical quality attribute is selected from the group consisting of oxidation, isomerization, deamidation, disulfide bond modification, and glycosylation. In some embodiments, the critical quality attribute is oxidation. In some embodiments, the two or more different types of antibodies have different critical quality attributes. In some embodiments, the two or more different types of antibodies have a common critical quality attribute.

In another aspect, provided are methods for determining a critical quality attribute of a co-formulation comprising an anti-PD-1 antibody and an anti-TIGIT antibody. In some embodiments, the method comprises (i) preparing a sample of the co-formulation; and (ii) performing a liquid chromatography-mass spectrometry (LC-MS) technique on the sample, wherein the LC-MS measures the critical quality attribute.

In some embodiments, the critical quality attribute of the anti-PD-1 antibody is an oxidation at an amino acid corresponding to M105 in SEQ ID NO: 8. In some embodiments, the critical quality attribute of the anti-PD-1 antibody is an oxidation at an amino acid corresponding to M105 in SEQ ID NO: 9. In some embodiments, the critical quality attribute of the anti-PD-1 antibody is an oxidation at an amino acid corresponding to M105 in SEQ ID NO: 10.

In some embodiments, the critical quality attribute of the anti-TIGIT antibody is an oxidation at an amino acid corresponding to W104 in SEQ ID NO: 28. In some embodiments, the critical quality attribute of the anti-TIGIT antibody is an oxidation at an amino acid corresponding to W104 in SEQ ID NO: 29. In some embodiments, the critical quality attribute of the anti-TIGIT antibody is an oxidation at an amino acid corresponding to W104 in SEQ ID NO: 30.

In another aspect, provided are methods for determining a critical quality attribute of a co-formulation comprising an anti-PD-1 antibody and an anti-LAG3 antibody. In some embodiments, the method comprises (i) preparing a sample of the co-formulation, and (ii) performing a liquid chromatography-mass spectrometry (LC-MS) technique on the sample, wherein the LC-MS measures the critical quality attribute.

In some embodiments, the critical quality attribute of the anti-PD-1 antibody is an oxidation at an amino acid corresponding to M105 in SEQ ID NO: 8. In some embodiments, the critical quality attribute of the anti-PD-1 antibody is an oxidation at an amino acid corresponding to M105 in SEQ ID NO: 9. In some embodiments, the critical quality attribute of the anti-PD-1 antibody is an oxidation at an amino acid corresponding to M105 in SEQ ID NO: 10.

In some embodiments, the critical quality attribute of the anti-LAG3 antibody is an oxidation at an amino acid corresponding to W102 in SEQ ID NO: 18. In some embodiments, the critical quality attribute of the anti-LAG3 antibody is an oxidation at an amino acid corresponding to W102 in SEQ ID NO: 19. In some embodiments, the critical quality attribute of the anti-LAG3 antibody is an oxidation at an amino acid corresponding to W102 in SEQ ID NO. 20.

In another aspect, provided are methods for determining a critical quality attribute of a co-formulation comprising an anti-PD-1 antibody and an anti-ILT4 antibody. In some embodiments, the method comprises (i) preparing a sample of the co-formulation; and (ii) performing a liquid chromatography-mass spectrometry (LC-MS) technique on the sample, wherein the LC-MS measures the critical quality attribute.

In some embodiments, the critical quality attribute of the anti-PD-1 antibody is an oxidation at an amino acid corresponding to M105 in SEQ ID NO: 8. In some embodiments, the critical quality attribute of the anti-PD-1 antibody is an oxidation at an amino acid corresponding to M105 in SEQ ID NO: 9. In some embodiments, the critical quality attribute of the anti-PD-1 antibody is an oxidation at an amino acid corresponding to M105 in SEQ ID NO: 10.

In some embodiments, the critical quality attribute of the anti-ILT4 antibody is an oxidation at an amino acid corresponding to W102 in SEQ ID NO: 112. In some embodiments, the critical quality attribute of the anti-ILT4 antibody is an oxidation at an amino acid corresponding to W102 in SEQ ID NO: 113. In some embodiments, the critical quality attribute of the anti-ILT4 antibody is an oxidation at an amino acid corresponding to W102 in SEQ ID NO: 114.

In some embodiments, the critical quality attribute of the anti-ILT4 antibody is an oxidation at an amino acid corresponding to W7 in SEQ ID NO: 113. In some embodiments, the critical quality attribute of the anti-LAG3 antibody is an oxidation at an amino acid corresponding to W7 in SEQ ID NO: 114.

In another aspect, provided are methods for determining a critical quality attribute of an anti-PD-1 antibody that comprises a light chain variable region comprising three light chain CDRs comprising CDRL1 of SEQ ID NO:1, CDRL2 of SEQ ID NO:2 and CDRL3 of SEQ ID NO:3 and a heavy chain variable region comprising three heavy chain CDRs of CDRH1 of SEQ ID NO:6, CDRH2 of SEQ ID NO:7 and CDRH3 of SEQ ID NO:8. In some embodiments, the method comprises (i) performing reduced-peptide mapping on a sample comprising the anti-PD-1 antibody, comprising digesting with Lys-C or trypsin; and (ii) performing a liquid chromatography-mass spectrometry (LC-MS) technique on a digested sample, wherein the LC-MS measures the critical quality attribute.

In some embodiments, the critical quality attribute of the anti-PD-1 antibody is an oxidation at an amino acid corresponding to M105 in SEQ ID NO: 10.

In some embodiments, the Lys-C or trypsin is mixed with the sample for about 30 mins to 90 mins at a temperature within a range of about 30° C. to 40° C.

In some embodiments, the method further comprises mixing a reducing agent solution with the sample. In some embodiments, the reducing agent solution comprises dithiothreitol. In some embodiments, a concentration of dithiothreitol in the reducing agent solution is within a range of about 100 μM to 300 μM. In some embodiments, the reducing agent solution is mixed with the sample for about 25 mins to 45 mins at a temperature within a range of about 25° C. to 40° C.

In some embodiments, the step of digesting comprises mixing an alkylating agent with the sample. In some embodiments, the alkylating agent comprises iodoacetamide. In some embodiments, the alkylating agent is mixed with the sample for about 25 mins to 45 mins.

In some embodiments, the analytical method comprises (i) applying the digested sample to a chromatography material; and (ii) eluting with a solution comprising a mobile phase A and a mobile phase B. In some embodiments, the mobile phase A comprises formic acid or trifluoracetic acid. In some embodiments, the mobile phase B comprises formic acid or trifluoracetic acid.

### BRIEF DESCRIPTION OF THE DRAWINGS

FIG. 1 illustrates analysis of critical quality attributes (CQAs) by liquid chromatography-mass spectrometry (LC-MS) of two antibodies that are co-formulated. Figure discloses SEQ ID NOS 115-116, respectively, in order of appearance.

FIG. 2A shows the results from reversed phase liquid chromatography (RPLC) separation of the intact co-formulated mAbs. FIG. 2B depicts the results from RPLC separation of the co-formulated mAbs using limited digestion with IdeS Protease followed by disulfide reduction. FIG. 2C shows hydrophobic interaction chromatography (HIC) separation of the co-formulated mAbs.

FIG. 3A shows electrospray ionization (ESI) charge state distributions of M105 (mAb-1)(top) and W104 (mAb-2) (bottom) by HRMS (Thermo Scientific™ Q Exactive™). FIG. 3B depicts ESI charge state distributions of M105 (mAb-1) (top) and W104 (mAb-2) (bottom) by QDa mass spectrometry. FIG. 3C shows MassLynx Software calculated theoretical m/z envelope (top) and isotope distributions of +5 charge state (bottom) of M105 (mAb-1). FIG. 3D depicts MassLynx Software calculated theoretical m/z envelope (top) and isotope distributions of +5 charge state (bottom) of W104 (mAb-2).

FIGS. 4A-4G are LC-MS chromatograms with selected ion recording (SIR) channels. FIG. 4A depicts Overlay of SIR channels 1-6. FIG. 4B shows SIR channel 1 for the native M105 peptide from mAb-1. FIG. 4C shows SIR channel 2 for oxidized M105 (+16 Da) peptide from mAb-1. FIG. 4D shows SIR channel 3 for native W104 peptide from mAb-2. FIG. 4E shows SIR channel 4 for W104 (+4 Da) peptide from mAb-2. FIG. 4F shows SIR channel 5 for W104 (+16 Da) peptide from mAb-2. FIG. 4G shows SIR channel 6 for W104 (+32 Da) peptide from mAb-2.

FIG. 5 is a fishbone diagram for QDa-based focused peptide mapping (FPM) method parameters in both sample preparation and instrument running. Critical parameters are highlighted

FIGS. 6A and 6B are scatterplots of mAb-1 M105 and mAb-2 W104% oxidation in co-formulated drug product. FIG. 6A shows Stage 1 DoE-1 robustness study and FIG. 6B shows Stage 1 DoE-2 robustness study. Dashed lines represent the ARR.

FIGS. 7A and 7B are prediction profiles of mAb-1 M105 and mAb-2 W104% oxidation in co-formulated drug product. FIG. 7A shows Stage 1 DoE-1 robustness study and FIG. 7B shows Stage 1 DoE-2 robustness study. Dashed lines represent the ARR.

FIGS. 8A-8C are scatterplots of mAb-1 M105 and mAb-2 W104% oxidation in co-formulated drug product from Stage 2 robustness study. FIG. 8A shows Probe Temperature. FIG. 8B shows Capillary Voltage. FIG. 8C shows Lys-C Lot. Dashed lines represent the ARR.

FIG. 9 depicts comparison of % oxidation calculated from the peak areas of the +5 charge state and the peak areas of the sum of all charge states.

FIG. 10 shows analysis of non-stressed and ICH light stressed co-formulated drug product (DP).

FIG. 11 shows QDa SIR channels 1-5 for mAb-4 non-oxidized and oxidized W102 components of interest from a non-stressed sample.

FIG. 12 shows QDa SIR channels 1-5 for mAb-4 non-oxidized and oxidized W102 peaks of interest from a 2X ICH light-stressed sample.

FIG. 13 shows QDa SIR channels 6-9 overlay for mAb-3 non-oxidized and oxidized M105 peaks of interest from a 2X ICH light-stressed sample.

FIG. 14 shows QDa SIR channels 1 and 2 for mAb-5 non-oxidized and oxidized M105 peaks of interest.

FIG. 15 shows QDa SIR channels 3-6 for mAb-6 non-oxidized and oxidized W102 peaks of interest.

FIG. 16 shows QDa SIR channels 7-10 for mAb-6 non-oxidized and oxidized W7 peaks of interest.

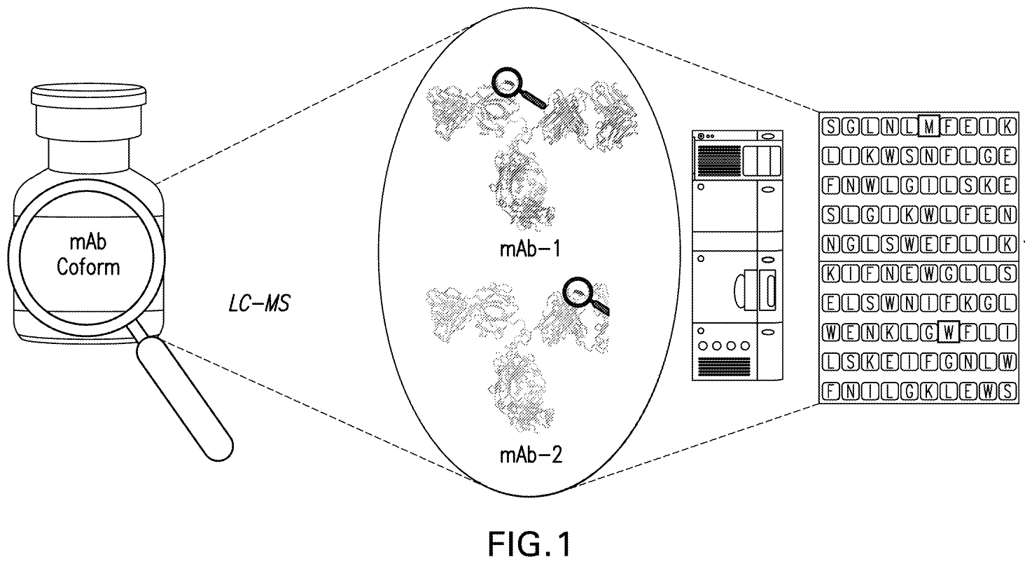

### DETAILED DESCRIPTIONS

In one aspect, the present disclosure provides methods for determining (to include in specific embodiments of the present invention, assessing, analyzing, detecting, monitoring and/or quantifying) a co-formulation during production. Also provided are methods comprising preparing a sample of a co-formulation comprising two or more different types of antibodies, and performing an analytical method on the sample to measures a critical quality attribute of each of the two or more different types of antibodies simultaneously.

The present disclosure is based, in part, on an insight that a co-formulation comprising two or more kinds of antibodies can create analytical challenges during development, for example, when the two antibodies have similar physicochemical properties and/or large concentration disparity. Analysis of PTMs in each antibody at release and during the stability studies is critical, for example, for assessing developability, screening formulations, and product quality. In some embodiments, oxidation of tryptophan and methionine residues, for example, those located in CDRs, can often be linked to changes in potency or other clinically relevant attributes with consequent classification as Critical Quality Attributes (CQA).

Characterization and quantitation of the oxidized amino acid residues can be performed via LC-MS analysis of digested proteins. While reduced peptide mapping (RPM) by RPLC coupled with high resolution mass spectrometry (HRMS) may provide selectivity and sensitivity, the method is often not quality control (QC)-friendly, and the method validation is challenging in both in-process control (IPC) and QC environments. Moreover, low throughput, high instrument cost, and need for well-trained personnel, also can impede implementation in commercial QC labs. In some embodiments. QDa-based LC-MS method may provide advantages in the robustness, high throughput, low cost, ease of use, and easy transfer to QC labs.

The present disclosure provides robust, reproducible, and/or sensitive methods for simultaneous detection of specific non-oxidized and oxidized peptides from two different antibodies in a co-formulation.

### 1. Definitions

Certain technical and scientific terms are specifically defined below. Unless specifically defined elsewhere in this document, all other technical and scientific terms used herein have the meaning commonly understood by one of ordinary skill in the art to which this disclosure relates.

“About” when used to modify a numerically defined parameter means that the parameter is within 20%, within 15%, within 10%, within 9% i4, within 8%, within 7%, within 6%, within 5%, within 4%, within 3%, within 2%, within 1%, or less of the stated numerical value or range for that parameter; where appropriate, the stated parameter may be rounded to the nearest whole number.

As used herein, including the appended claims, the singular forms of words such as “a,” “an,” and “the,” include their corresponding plural references unless the context clearly dictates otherwise.

As used herein, the term “critical quality attribute” refers to a property or characteristic that should be within an appropriate limit, range, or distribution to ensure the desired product quality. In some embodiments, a critical quality attribute may be a physical, chemical, biological, or microbiological property or characteristic. In some embodiments, a critical quality attribute is a level of oxidation, isomerization, deamidation, disulfide bond modification, or glycosylation. In specific embodiments, the critical quality attribute is oxidation.

As used herein, the term “determining” refers to quantitative or qualitative analysis of one or more CQAs via, for example, spectroscopy, chromatography, and the like. In some embodiments, “determining” includes assessing, analyzing, detecting, monitoring and/or quantifying.

As used herein, the term “reducing agent” refers to any compound or mixture of compounds that is capable of reducing another compound. In this process the proportion of hydrogen is increased while the proportion of oxygen is decreased and the number of multiple bonds is decreased. In some embodiments, the term encompasses any agent that is capable of unfolding proteins from their native configurations, e.g., by breaking disulfide bonds.

As used herein, the term “analytical method” refers to all laboratory methods and protocols that are used to identify, quantify, distinguish or characterize CQA(s). In some embodiments, analytical methods may include liquid chromatography, gas chromatography, gel electrophoresis, mass spectrometry (MS), densitometry, colorimetrics, spectrophotometry, energy magnetic radiation, nuclear magnetic resonance (NMR), and combinations thereof. In some embodiments, analytical methods may include liquid chromatography-mass spectrometry (LC-MS), hydrophobic interaction chromatography (HIC) and/and reverse phase liquid chromatography (RPLC).

As used herein, the term “antibody” refers to any form of immunoglobulin molecule that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic. As used herein, the term “antibody” encompasses not only intact polyclonal or monoclonal antibodies, but also, unless otherwise specified, fusion proteins comprising an antigen binding portion, and any other modified configuration of the immunoglobulin molecule that comprises an antigen binding fragment thereof that competes with the intact antibody for specific binding.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

“Variable regions” or “V region” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may be referred to as “V_{H}.” The variable region of the light chain may be referred to as “V_{L}”.

Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. As referred to herein the light chain CDRs are CDRL1, CDRL2 and CDRL3, respectively, and the heavy chain CDRs are CDRH1, CDRH2 and CDRH3, respectively. The assignment of amino acids to each domain is, generally, in accordance with the definitions of Sequences of Proteins of Immunological Interest, Kabat, et al.; National Institutes of Health, Bethesda, Md; 5th ed.; NIH PubL. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

A “CDR” refers to one of three hypervariable regions (H1, H2, or H3) within the non-framework region of the antibody VH β-sheet framework, or one of three hypervariable regions (L1, L2, or L3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved b-sheet framework, and thus are able to adapt to different conformation. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art and shown below in Table 1. In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system.

TABLE 1
Correspondence between the CDR Numbering Systems

| | Kabat + | | | | | |
|---|---|---|---|---|---|---|
| | Chothia | IMGT | Kabat | AbM | Chothia | Contact |
| VH CDR1 | 26-35 | 27-38 | 31-35 | 26-35 | 26-32 | 30-35 |
| VH CDR2 | 50-65 | 56-65 | 50-65 | 50-58 | 52-56 | 47-58 |
| VH CDR3 | 95-102 | 105-117 | 95-102 | 95-102 | 95-102 | 93-101 |
| VL CDRI | 24-34 | 27-38 | 24-34 | 24-34 | 24-34 | 30-36 |
| VL CDR2 | 50-56 | 56-65 | 50-56 | 50-56 | 50-56 | 46-55 |
| VL CDR3 | 89-97 | 105-117 | 89-97 | 89-97 | 89-97 | 89-96 |

“Chimeric antibody” refers to an antibody in which a portion of the heavy and/or light chain contains sequences derived from a particular species (e.g., human) or belonging to a particular antibody class or subclass, while the remainder of the chain(s) is derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences or derivatives thereof. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences or derivatives thereof, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” may be added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used herein, unless otherwise indicated, “antibody fragment” or “antigen binding fragment” refers to a fragment of an antibody that retains the ability to bind specifically to the antigen, e.g., fragments that retain one or more CDR regions and the ability to bind specifically to the antigen. An antibody that “specifically binds to” PD-1, TIGIT, LAG3 or ILT4 is an antibody that exhibits preferential binding to PD-1, TIGIT, LAG3 or ILT4 (as appropriate) as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, will bind to the target protein with an affinity that is at least two-fold greater, preferably at least ten times greater, more preferably at least 20-times greater, and most preferably at least 100-times greater than the affinity with non-target proteins.

Antigen binding portions include, for example, Fab, Fab′, F(ab′)2, Fd′, Fv, fragments including CDRs, and single chain variable fragment antibodies (scFv), and polypeptides that contain at least a portion of an immunoglobulin that is sufficient to confer specific antigen binding to the antigen (e.g., PD-1, TIGIT, LAG3 or ILT4). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, igD, IgE, IgG, and IgM, and several of these may be further divided into subclasses (isotypes), e.g., IgG1, IgG2, IgG3, IgG4, IgA1, and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

As used herein, the terms “at least one” item or “one or more” item each include a single item selected from the list as well as mixtures of two or more items selected from the list.

The term “variant” when used in relation to an antibody or an amino acid region within the antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a variant of an anti-PD-1 antibody may result from one or more (such as, for example, about 1 to about 25, about 1 to about 20, about 1 to about 15, about 1 to about 10, or about 1 to about 5) changes to an amino acid sequence of a native or previously unmodified antibody. Variants may be naturally occurring or may be artificially constructed. Polypeptide variants may be prepared from the corresponding nucleic acid molecules encoding the variants. In specific embodiments, an antibody variant at least retains the antibody functional activity. In some embodiments, an anti-PD-1 antibody variant binds to PD-1 and/or is antagonistic to PD-1 activity. In specific embodiments, an anti-TIGIT antibody variant binds to TIGIT and/or is antagonistic to TIGIT activity. In specific embodiments, an anti-LAG3 antibody variant binds to LAG3 and/or is antagonistic to LAG3 activity. In specific embodiments, an anti-ILT4 antibody variant binds to ILT4 and/or is antagonistic to ILT4 activity.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 2 below.

TABLE 2
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared 100. For example, if 8 of 10 of the positions in two sequences are matched when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul. S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al, (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, NatI. Biomed. Res. Found., Washington, DC: Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

“Co-formulated” or “co-formulation” or “coformulation” or “coformulated” as used herein refers to at least two different antibodies or antigen binding fragments thereof which are formulated together and stored as a combined product in a single vial or vessel (for example an injection device) rather than being formulated and stored individually and then mixed before administration or separately administered. In one embodiment, the co-formulation contains two different antibodies or antigen binding fragments thereof.

The term “pharmaceutically acceptable carrier” refers to any inactive substance that is suitable for use in a formulation for the delivery of a therapeutic agent. A carrier may be an anti-adherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like), dextrose, vegetable oils (such as olive oil), saline, buffer, buffered saline, and isotonic agents such as sugars, polyalcohols, sorbitol, and sodium chloride.

It is understood that wherever embodiments are described herein with the language “comprising,” otherwise analogous embodiments described in terms of “consisting of” and/or “consisting essentially of” are also provided.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. Numerical values provided herein, and the use of the term “about”, may include variations of ±1%, ±2%, ±3%, ±4%, ±5%, ±10%, ±15%, and ±20% and their numerical equivalents. All ranges also are intended to include all included sub-ranges, although not necessarily explicitly set forth. For example, a range of 3 to 7 days is intended to include 3, 4, 5, 6, and 7 days. In addition, the term “or,” as used herein, denotes alternatives that may, where appropriate, be combined; that is, the term “or” includes each listed alternative separately as well as their combination.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members.

The present disclosure also envisages the explicit exclusion of one or more of any of the group members in the claims.

Unless otherwise defined, all technical and scientific terms used herein have the same meaning as commonly understood by one of ordinary skill in the art to which this disclosure relates. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g” or “for example” is not meant to be exhaustive or limiting.

Exemplary methods and materials are described herein, although methods and materials similar or equivalent to those described herein can also be used in the practice or testing of the present disclosure. The materials, methods, and examples are illustrative only and not intended to be limiting.

### 2. Anti-PD-1 Antibodies and Antigen Binding Fragments Thereof

Provided herein are anti-PD-1 antibodies or antigen binding fragments thereof that can be used in the various methods disclosed herein. Any antibodies that bind to a PD-1 polypeptide, a PD-1 polypeptide fragment, a PD-1 peptide, or a PD-1 epitope and block the interaction between PD-1 and its ligand PD-L1 or PD-L2 can be used. In some embodiments, the anti-PD-1 antibody binds to a PD-1 polypeptide, a PD-1 polypeptide fragment, a PD-1 peptide, or a PD-1 epitope and blocks the interaction between PD-1 and PD-L1. In other embodiments, the anti-PD-1 antibody binds to a PD-1 polypeptide, a PD-1 polypeptide fragment, a PD-1 peptide, or a PD-1 epitope and blocks the interaction between PD-1 and PD-L2. In yet other embodiments, the anti-PD-1 antibody binds to a PD-1 polypeptide, a PD-1 polypeptide fragment, a PD-1 peptide, or a PD-1 epitope and blocks the interaction between PD-1 and PD-L1 and the interaction between PD-1 and PD-L2.

In certain embodiments of various methods provided herein, the anti-PD-1 antibody or antigen binding fragment thereof comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively.

In some embodiments of various methods provided herein, the anti-PD-1 antibody or antigen binding fragment thereof comprises a V_{L} region comprising an amino acid sequence as set forth in SEQ ID NO:4, and a V_{H} region comprising an amino acid sequence as set forth in SEQ ID NO:9.

In other embodiments of various methods provided herein, the anti-PD-1 antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10.

In certain embodiments of various methods provided herein, the anti-PD-1 antibody or antigen binding fragment thereof comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively.

In some embodiments of various methods provided herein, the anti-PD-1 antibody or antigen binding fragment thereof comprises a V_{L} region comprising an amino acid sequence as set forth in SEQ ID NO:34, and a V_{H} region comprising an amino acid sequence as set forth in SEQ ID NO:39.

In other embodiments of various methods provided herein, the anti-PD-1 antibody or antigen binding fragment thereof comprises a light chain comprising or consisting of an amino acid sequence as set forth in SEQ ID NO:35 and a heavy chain comprising or consisting of an amino acid sequence as set forth in SEQ ID NO:40.

In various embodiments, the anti-PD-1 antibody or antigen binding fragment thereof comprises a variant of the amino acid sequences disclosed herein. A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the anti-PD-1 antibody or antigen binding fragment thereof has a V_{L} domain with at least 95%, 90%, 85%, 80%, 75% or 50% sequence homology to one of the V_{L} domains described herein, and exhibits specific binding to PD-1. In another embodiment, the anti-PD-1 antibody or antigen binding fragment thereof has a V_{H} domain with at least 95%, 90%, 85%, 80%, 75% or 50% sequence homology to one of the V_{H} domains described herein, and exhibits specific binding to PD-1. In yet another embodiment, the anti-PD-1 antibody or antigen binding fragment thereof has a V_{L} domain with at least 95%, 90%, 85%, 80%, 75% or 50% sequence homology to one of the V_{L} domains described herein and a V_{H} domain with at least 95%, 90%, 85%, 80%, 75% or 50% sequence homology to one of the V_{H} domains described herein, and exhibits specific binding to PD-1.

In one embodiment, the anti-PD-1 antibody or antigen binding fragment thereof has a V_{L} domain having up to 1, 2, 3, 4, 5 or more amino acid substitutions, deletions and/or additions in one of the V_{L} domains described herein, and exhibits specific binding to PD-1. In another embodiment, the anti-PD-1 antibody or antigen binding fragment thereof has a V_{H} domain having up to 1, 2, 3, 4, 5 or more amino acid substitutions, deletions, and/or additions in one of the V_{H} domains described herein, and exhibits specific binding to PD-1. In yet another embodiment, the anti-PD-1 antibody or antigen binding fragment thereof has a V_{L} domain having up to 1, 2, 3, 4, 5 or more amino acid substitutions, deletions, and/or additions in one of the V_{L} domains described herein and a V_{H} domain having up to 1, 2, 3, 4, 5 or more amino acid substitutions, deletions, and/or additions in one of the V_{H} domains described herein, and exhibits specific binding to PD-1.

In various embodiments, the anti-PD-1 antibody or antigen binding fragment thereof is selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances, an IgG4 constant domain, for example, may be used. In various embodiments, the heavy chain constant domain contains one or more amino acid mutations (e.g., IgG4 with S228P mutation) to generate desired characteristics of the antibody. These desired characteristics include but are not limited to modified effector functions, physical or chemical stability, half-life of antibody, etc.

Ordinarily, amino acid sequence variants of the anti-PD-1 antibodies and antigen binding fragments thereof disclosed herein will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3. M. O. Dayhoff (ed.), pp. 353-358, Natl. Biomed. Res. Found., Washington, DC; Altschul, S. F., (1991) J. Mol. Biol. 219.555-565; States, D. J., et al., (1991) Methods 3.66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS. Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

In some embodiments, the anti-PD-1 antibody is a human antibody. In other embodiments, the anti-PD-1 antibody is a humanized antibody.

In some embodiments, the heavy chain of the anti-PD-1 antibody has a human IgG1 backbone. In other embodiments, the heavy chain of the anti-PD-1 antibody has a human IgG2 backbone. In yet other embodiments, the heavy chain of the anti-PD-1 antibody has a human IgG3 backbone. In still other embodiments, the heavy chain of the anti-PD-1 antibody has a human IgG4 backbone.

In some embodiments, the heavy chain of the anti-PD-1 antibody has a human IgG1 variant backbone. In other embodiments, the heavy chain of the anti-PD-1 antibody has a human IgG2 variant backbone. In yet other embodiments, the heavy chain of the anti-PD-1 antibody has a human IgG3 variant backbone. In still other embodiments, the heavy chain of the anti-PD-1 antibody has a human IgG4 variant (e.g., IgG4 with S228P mutation) backbone.

In certain embodiments, the anti-PD-1 antibody is selected from the group consisting of pembrolizumab, nivolumab, cemiplimab, pidilizumab, AMP-514, PDR001, BGB-A317, and MGA012. In one embodiment, the anti-PD-1 antibody is pembrolizumab. In another embodiment, the anti-PD-1 antibody is nivolumab. In another embodiment, the anti-PD-1 antibody is cemiplimab. In yet another embodiment, the anti-PD-1 antibody is pidilizumab. In one embodiment, the anti-PD-1 antibody is AMP-514. In another embodiment, the anti-PD-1 antibody is PDR001. In yet another embodiment, the anti-PD-1 antibody is BGB-A317. In still another embodiment, the anti-PD-1 antibody is MGA012.

In some embodiments, the anti-PD-1 antibody can be any antibody, antigen binding fragment thereof, or variant thereof disclosed in WO 2008/156712, the disclosure of which is incorporated by reference herein in its entirety.

### 3. Anti-TIGIT Antibodies and Antigen Binding Fragments Thereof

In addition, provided herein are anti-TIGIT antibodies or antigen binding fragments thereof that can be used in the various methods disclosed herein. Any antibodies that bind to a TIGIT polypeptide, a TIGIT polypeptide fragment, a TIGIT peptide, or a TIGIT epitope and block the interaction between TIGIT and its ligand CD155 and/or CD112 can be used. In some embodiments, the anti-TIGIT antibody binds to a TIGIT polypeptide, a TIGIT polypeptide fragment, a TIGIT peptide, or a TIGIT epitope and blocks the interaction between TIGIT and CD155. In other embodiments, the anti-TIGIT antibody binds to a TIGIT polypeptide, a TIGIT polypeptide fragment, a TIGIT peptide, or a TIGIT epitope and blocks the interaction between TIGIT and CD112. In yet other embodiments, the anti-TIGIT antibody binds to a TIGIT polypeptide, a TIGIT polypeptide fragment, a TIGIT peptide, or a TIGIT epitope and blocks the interaction between TIGIT and CD155 and the interaction between TIGIT and CD112.

In certain embodiments of various methods provided herein, the anti-TIGIT antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In some embodiments of various methods provided herein, the anti-TIGIT antibody or antigen binding fragment thereof comprises a V_{L} region comprising an amino acid sequence as set forth in SEQ ID NO:24, and a V_{H} region comprising an amino acid sequence as set forth in SEQ ID NO:29.

In other embodiments of various methods provided herein, the anti-TIGIT antibody or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.

In certain embodiments of various methods provided herein, the anti-TIGIT antibody or antigen binding fragment thereof comprises a V_{L} CDR1 comprising the amino acid sequence as set forth in SEQ ID NO:21, a V_{L} CDR2 comprising the amino acid sequence as set forth in one of SEQ ID NOS:22 or 92-104, a V_{L} CDR3 comprising the amino acid sequence as set forth in SEQ ID NO:23, and a V_{H} CDR1 comprising the amino acid sequence as set forth in SEQ ID NO:26, a V_{H} CDR2 comprising the amino acid sequence as set forth in one of SEQ ID NOS:27 or 58-76, a V_{H} CDR3 comprising the amino acid sequence as set forth in one of SEQ ID NOS:28 or 77-91.

In certain embodiments of various methods provided herein, the anti-TIGIT antibody or antigen binding fragment thereof comprises a V_{L} CDR1 comprising the amino acid sequence as set forth in SEQ ID NO:21; a V_{L} CDR2 comprising the amino acid sequence as set forth in SEQ ID NO:104 (X₁X₂KTLAE), wherei X₁ is N, A, V, W, S, T, R, H, G, I, or V, and wherein X₂ is A, N, I, L, T, or V; a V_{L} CDR3 comprising the amino acid sequence as set forth in SEQ ID NO:23; a V_{H} CDR1 comprising the amino acid sequence as set forth in SEQ ID NO:26; a V_{H} CDR2 comprising the amino acid sequence as set forth in SEQ ID NO:76 (YIDPYNX₇X₈AKYX₁₂X₁₃KFX₁₆G), wherein X₇ is D, R, L, K, F, S, Y or V, wherein X₈ is G, R, N, Q, E, L K, S, Y or V, wherein X₁₂ is N, A or S, wherein X₁₃ is E or Q, and wherein X₁₆ is K or Q; and a V_{H} CDR3 comprising the amino acid sequence as set forth in SEQ ID NO:91 (GGPYGX₆YFDV), wherein X₆ is W, A, D, E, F, G, I, K, N, Q, R, S, T, V or Y.

In certain embodiments of various methods provided herein, the anti-TIGIT antibody or antigen binding fragment thereof comprises a V_{L} that comprises a V_{L} CDR1, a V_{L} CDR2, and a V_{L} CDR3, and a V_{H} that comprises a V_{H} CDR1, a V_{H} CDR2, and a V_{H} CDR3; the V_{L} CDR1 comprising the amino acid sequence as set forth in SEQ ID NO:21; the V_{L} CDR2 comprising the amino acid sequence as set forth in one of SEQ LD NOS:22 or 92-104; the V_{L} CDR3 comprising the amino acid sequence as set forth in SEQ ID NO:23; the V_{H} CDR1 comprising the amino acid sequence as set forth in SEQ ID NO:26; the V_{H} CDR2 comprising the amino acid sequence as set forth in one of SEQ ID NOS:27 or 58-76; the V_{H} CDR3 comprising the amino acid sequence as set forth in one of SEQ ID NOS:28 or 77-91.

In certain embodiments of various methods provided herein, the anti-TIGIT antibody or antigen binding fragment thereof comprises a V_{L} that comprises a V_{L} CDR1, a V_{L} CDR2, and a V_{L} CDR3, and a V_{H} that comprises a V_{H} CDR1, a V_{H} CDR2, and a V_{H} CDR3; the V_{L} CDR1 comprising the amino acid sequence as set forth in SEQ ID NO:21; the V_{L} CDR2 comprising the amino acid sequence as set forth in SEQ ID NO:104 (X₁X₂KTLAE), wherein X₁ is N, A, V, W, S, T, R, H, G, I, or V, and wherein X₂ is A, N, I, L, T, or V; the V_{L} CDR3 comprising the amino acid sequence as set forth in SEQ ID NO:23; the V_{H} CDR1 comprising the amino acid sequence as set forth in SEQ ID NO:26; the V_{H} CDR2 comprising the amino acid sequence as set forth in SEQ ID NO:76 (YIDPYNX₇X₈AKYX₁₂X₁₃KFX₁₆G), wherein X, is D, R, L, K, F, S, Y or V, wherein X₈ is G, R, N, Q, E, L K, S, Y or V, wherein X₁₂ is N, A or S, wherein X₁₃ is E or Q, and wherein X₁₆ is K or Q; and the V_{H} CDR3 comprising the amino acid sequence as set forth in SEQ ID NO:91 (GGPYGX₆YFDV), wherein X₆ is W, A, D, E, F, G, I, K, N, Q, R, S, T, V or Y.

In certain embodiments of various methods provided herein, the anti-TIGIT antibody or antigen binding fragment thereof comprises a light chain that comprises a V_{L} CDR1, a V_{L} CDR2, and a V_{L} CDR3, and a heavy chain that comprises a V_{H} CDR1, a V_{H} CDR2, and a V_{H} CDR3; the V_{L} CDR1 comprising the amino acid sequence as set forth in SEQ ID NO:21; the V_{L} CDR2 comprising the amino acid sequence as set forth in one of SEQ ID NOS:22 or 92-104: the V_{L} CDR3 comprising the amino acid sequence as set forth in SEQ ID NO:23; the V_{H} CDR1 comprising the amino acid sequence as set forth in SEQ ID NO:26; the V_{H} CDR2 comprising the amino acid sequence as set forth in one of SEQ ID NOS:27 or 58-76: the V_{H} CDR3 comprising the amino acid sequence as set forth in one of SEQ ID NOS:28 or 77-91.

In certain embodiments of various methods provided herein, the anti-TIGIT antibody or antigen binding fragment thereof comprises a light chain that comprises a V_{L} CDR1, a V_{L} CDR2, and a V_{L} CDR3, and a heavy chain that comprises a V_{H} CDR1, a V_{H} CDR2, and a V_{H} CDR3: the V_{L} CDR1 comprising the amino acid sequence as set forth in SEQ ID NO:21; the V_{L} CDR2 comprising the amino acid sequence as set forth in SEQ ID NO:104 (X₁X₂KTLAE), wherein X₁ is N, A, V, W, S, T, R, H, G, I, or V, and wherein X₂ is A, N, I, L, T, or V; the V_{L} CDR3 comprising the amino acid sequence as set forth in SEQ ID NO:23; the V_{H} CDR1 comprising the amino acid sequence as set forth in SEQ ID NO:26: the V_{H} CDR2 comprising the amino acid sequence as set forth in SEQ ID NO:76 (YIDPYNX₇X₈AKYX₁₂X₁₃KFX₁₆G), wherein X₇ is D, R, L, K, F, S, Y or V, wherein X₈ is G, R, N, Q, E, L K, S. Y or V, wherein X₁₂ is N. A or S, wherein X₁₃ is E or Q, and wherein X₁, is K or Q; and the V_{H} CDR3 comprising the amino acid sequence as set forth in SEQ ID NO:91 (GGPYGX₆YFDV), wherein X₆ is W, A, D, E, F, G, I, K, N, Q, R, S, T, V or Y.

In certain embodiments of various methods provided herein, the V_{H} CDR2 of the anti-TIGIT antibody or antigen binding fragment thereof comprises the amino acid sequence as set forth in SEQ ID NO:58. In some embodiments, the V_{H} CDR2 of the anti-TIGIT antibody or antigen binding fragment thereof comprises the amino acid sequence as set forth in SEQ ID NO:59. In other embodiments, the V_{H} CDR2 of the anti-TIGIT antibody or antigen binding fragment thereof comprises the amino acid sequence as set forth in SEQ ID NO:60. In yet other embodiments, the V_{H} CDR2 of the anti-TIGIT antibody or antigen binding fragment thereof comprises the amino acid sequence as set forth in SEQ ID NO:61. In certain embodiments, the V_{H} CDR2 of the anti-TIGIT antibody or antigen binding fragment thereof comprises the amino acid sequence as set forth in SEQ ID NO:62. In some embodiments, the V_{H} CDR2 of the anti-TIGIT antibody or antigen binding fragment thereof comprises the amino acid sequence as set forth in SEQ ID NO:63. In other embodiments, the V_{H} CDR2 of the anti-TIGIT antibody or antigen binding fragment thereof comprises the amino acid sequence as set forth in SEQ LD NO:64. In yet other embodiments, the V_{H} CDR2 of the anti-TIGIT antibody or antigen binding fragment thereof comprises the amino acid sequence as set forth in SEQ ID NO:65. In certain embodiments, the V_{H} CDR2 of the anti-TIGIT antibody or antigen binding fragment thereof comprises the amino acid sequence as set forth in SEQ ID NO:66. In some embodiments, the V_{H} CDR2 of the anti-TIGIT antibody or antigen binding fragment thereof comprises the amino acid sequence as set forth in SEQ ID NO:67. In other embodiments, the V_{H}CDR2 of the anti-TIGIT antibody or antigen binding fragment thereof comprises the amino acid sequence as set forth in SEQ ID NO:68. In yet other embodiments, the V_{H} CDR2 of the anti-TIGIT antibody or antigen binding fragment thereof comprises the amino acid sequence as set forth in SEQ ID NO:69. In certain embodiments, the V_{H} CDR2 of the anti-TIGIT antibody or antigen binding fragment thereof comprises the amino acid sequence as set forth in SEQ ID NO:70. In some embodiments, the V_{H} CDR2 of the anti-TIGIT antibody or antigen binding fragment thereof comprises the amino acid sequence as set forth in SEQ ID NO:71. In other embodiments, the V_{H} CDR2 of the anti-TIGIT antibody or antigen binding fragment thereof comprises the amino acid sequence as set forth in SEQ ID NO:72. In yet other embodiments, the V_{H} CDR2 of the anti-TIGIT antibody or antigen binding fragment thereof comprises the amino acid sequence as set forth in SEQ ID NO:73. In certain embodiments, the V_{H} CDR2 of the anti-TIGIT antibody or antigen binding fragment thereof comprises the amino acid sequence as set forth in SEQ ID NO:74. In some embodiments, the V_{H} CDR2 of the anti-TIGIT antibody or antigen binding fragment thereof comprises the amino acid sequence as set forth in SEQ ID NO:75.

In certain embodiments of various methods provided herein, the V_{H} CDR3 of the anti-TIGIT antibody or antigen binding fragment thereof comprises the amino acid sequence as set forth in SEQ ID NO:77. In some embodiments, the V_{H} CDR3 of the anti-TIGIT antibody or antigen binding fragment thereof comprises the amino acid sequence as set forth in SEQ ID NO:78. In other embodiments, the V_{H} CDR3 of the anti-TIGIT antibody or antigen binding fragment thereof comprises the amino acid sequence as set forth in SEQ ID NO:79. In yet other embodiments, the V_{H} CDR3 of the anti-TIGIT antibody or antigen binding fragment thereof comprises the amino acid sequence as set forth in SEQ ID NO:80. In certain embodiments, the V_{H} CDR3 of the anti-TIGIT antibody or antigen binding fragment thereof comprises the amino acid sequence as set forth in SEQ ID NO:81. In some embodiments, the V_{H} CDR3 of the anti-TIGIT antibody or antigen binding fragment thereof comprises the amino acid sequence as set forth in SEQ ID NO:82. In other embodiments, the V_{H} CDR3 of the anti-TIGIT antibody or antigen binding fragment thereof comprises the amino acid sequence as set forth in SEQ ID NO:83. In yet other embodiments, the V_{H} CDR3 of the anti-TIGIT antibody or antigen binding fragment thereof comprises the amino acid sequence as set forth in SEQ ID NO:84. In certain embodiments, the V_{H} CDR3 of the anti-TIGIT antibody or antigen binding fragment thereof comprises the amino acid sequence as set forth in SEQ ID NO:85. In some embodiments, the V_{H} CDR3 of the anti-TIGIT antibody or antigen binding fragment thereof comprises the amino acid sequence as set forth in SEQ ID NO:86. In other embodiments, the V_{H} CDR3 of the anti-TIGIT antibody or antigen binding fragment thereof comprises the amino acid sequence as set forth in SEQ ID NO:87. In yet other embodiments, the V_{H} CDR3 of the anti-TIGIT antibody or antigen binding fragment thereof comprises the amino acid sequence as set forth in SEQ ID NO:88. In certain embodiments, the V_{H} CDR3 of the anti-TIGIT antibody or antigen binding fragment thereof comprises the amino acid sequence as set forth in SEQ ID NO:89. In some embodiments, the V_{H} CDR3 of the anti-TIGIT antibody or antigen binding fragment thereof comprises the amino acid sequence as set forth in SEQ ID NO:90.

In certain embodiments of various methods provided herein, the V_{L} CDR2 of the anti-TIGIT antibody or antigen binding fragment thereof comprises the amino acid sequence as set forth in SEQ ID NO:92. In some embodiments, the V_{L} CDR2 of the anti-TIGIT antibody or antigen binding fragment thereof comprises the amino acid sequence as set forth in SEQ ID NO:93. In other embodiments, the V_{L} CDR2 of the anti-TIGIT antibody or antigen binding fragment thereof comprises the amino acid sequence as set forth in SEQ ID NO:94. In yet other embodiments, the V_{L} CDR2 of the anti-TIGIT antibody or antigen binding fragment thereof comprises the amino acid sequence as set forth in SEQ ID NO:95. In certain embodiments, the V_{L} CDR2 of the anti-TIGIT antibody or antigen binding fragment thereof comprises the amino acid sequence as set forth in SEQ ID NO:96. In some embodiments, the V_{L} CDR2 of the anti-TIGIT antibody or antigen binding fragment thereof comprises the amino acid sequence as set forth in SEQ ID NO:97. In other embodiments, the V_{L} CDR2 of the anti-TIGIT antibody or antigen binding fragment thereof comprises the amino acid sequence as set forth in SEQ ID NO:98. In yet other embodiments, the V_{L} CDR2 of the anti-TIGIT antibody or antigen binding fragment thereof comprises the amino acid sequence as set forth in SEQ ID NO:99. In certain embodiments, the V_{L} CDR2 of the anti-TIGIT antibody or antigen binding fragment thereof comprises the amino acid sequence as set forth in SEQ ID NO:100. In some embodiments, the V_{L} CDR2 of the anti-TIGIT antibody or antigen binding fragment thereof comprises the amino acid sequence as set forth in SEQ ID NO:101. In other embodiments, the V_{L} CDR2 of the anti-TIGIT antibody or antigen binding fragment thereof comprises the amino acid sequence as set forth in SEQ ID NO:102. In yet other embodiments, the V_{L} CDR2 of the anti-TIGIT antibody or antigen binding fragment thereof comprises the amino acid sequence as set forth in SEQ ID NO:103.

In various embodiments, the anti-TIGIT antibody or antigen binding fragment thereof comprises a variant of the amino acid sequences disclosed herein. A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the anti-TIGIT antibody or antigen binding fragment thereof has a V_{L} domain with at least 95%, 90%, 85%, 80%, 75% or 50% sequence homology to one of the V_{L} domains described herein, and exhibits specific binding to TIGIT. In another embodiment, the anti-TIGIT antibody or antigen binding fragment thereof has a V_{H} domain with at least 95%, 90%, 85%, 80%, 75% or 50% sequence homology to one of the V_{H} domains described herein, and exhibits specific binding to TIGIT. In yet another embodiment, the anti-TIGIT antibody or antigen binding fragment thereof has a V_{L} domain with at least 95%, 90%, 85%, 80%, 75% or 50% sequence homology to one of the V_{L} domains described herein and a V_{H} domain with at least 95%, 90%, 85%, 80%, 75% or 50% sequence homology to one of the V_{H} domains described herein, and exhibits specific binding to TIGIT.

In one embodiment, the anti-TIGIT antibody or antigen binding fragment thereof has a V_{L} domain having up to 1, 2, 3, 4, 5 or more amino acid substitutions, deletions and/or additions in one of the V_{L} domains described herein, and exhibits specific binding to TIGIT. In another embodiment, the anti-TIGIT antibody or antigen binding fragment thereof has a V_{H} domain having up to 1, 2, 3, 4, 5 or more amino acid substitutions, deletions, and/or additions in one of the V_{H} domains described herein, and exhibits specific binding to TIGIT. In yet another embodiment, the anti-TIGIT antibody or antigen binding fragment thereof has a V_{L} domain having up to 1, 2, 3, 4, 5 or more amino acid substitutions, deletions, and/or additions in one of the V_{L} domains described herein and a V_{H} domain having up to 1, 2, 3, 4, 5 or more amino acid substitutions, deletions, and/or additions in one of the V_{H} domains described herein, and exhibits specific binding to TIGIT.

In various embodiments, the anti-TIGIT antibody or antigen binding fragment thereof is selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances, an IgG4 constant domain, for example, may be used. In various embodiments, the heavy chain constant domain contains one or more amino acid mutations (e.g., IgG4 with S228P mutation) to generate desired characteristics of the antibody. These desired characteristics include but are not limited to modified effector functions, physical or chemical stability, half-life of antibody, etc.

Ordinarily, amino acid sequence variants of the anti-TIGIT antibodies and antigen binding fragments thereof disclosed herein will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3. M. O. Dayhoff (ed.), pp. 353-358, Natl. Biomed. Res. Found., Washington, DC; Altschul, S. F., (1991) J. Mol. Biol. 219.555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300, ALIGNMENT STATISTICS: Karlin, S., et al., (1990) Proc. Natl. Acad. Sci. USA 87.2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

In some embodiments, the anti-TIGIT antibody is a human antibody. In other embodiments, the anti-TIGIT antibody is a humanized antibody.

In some embodiments, the heavy chain of the anti-TIGIT antibody has a human IgG1 backbone. In other embodiments, the heavy chain of the anti-TIGIT antibody has a human IgG2 backbone. In yet other embodiments, the heavy chain of the anti-TIGIT antibody has a human IgG3 backbone. In still other embodiments, the heavy chain of the anti-TIGIT antibody has a human IgG4 backbone.

In some embodiments, the heavy chain of the anti-TIGIT antibody has a human IgG1 variant backbone. In other embodiments, the heavy chain of the anti-TIGIT antibody has a human IgG2 variant backbone. In yet other embodiments, the heavy chain of the anti-TIGIT antibody has a human IgG3 variant backbone. In still other embodiments, the heavy chain of the anti-TIGIT antibody has a human IgG4 variant (e.g., IgG4 with S228P mutation) backbone.

In certain embodiments, the anti-TIGIT antibody is selected from the group consisting of BMS-986207, OMP-313M32, MTIG7192A (RG6058) and PTZ-201 (ASP8374). In one embodiment, the anti-TIGIT antibody is BMS-986207. In another embodiment, the anti-TIGIT antibody is OMP-313M32. In yet another embodiment, the anti-TIGIT antibody is MTIG7192A (RG6058). In still another embodiment, the anti-TIGIT antibody is PTZ-201 (ASP8374).

In some embodiments, the anti-TIGIT antibody can be any antibody, antigen binding fragment thereof, or variant thereof disclosed in WO 2016/028656 and WO 2017/030823, the disclosures of which are incorporated by reference herein in their entireties.

### 4. Anti-LAG3 Antibodies and Antigen Binding Fragments Thereof

Also provided herein are anti-LAG3 antibodies or antigen binding fragments thereof that can be used in the various methods disclosed herein. Any antibodies that bind to a LAG3 polypeptide, a LAG3 polypeptide fragment, a LAG3 peptide, or a LAG3 epitope and block the interaction between LAG3 and its ligand Class II MHC can be used.

In certain embodiments of various methods provided herein, the anti-LAG3 antibody or antigen binding fragment thereof comprises a V_{L} CDR1, a V_{L} CDR2, and a V_{L} CDR3 comprising amino acid sequences as set forth in SEQ ID NOS: 11, 12, and 13, respectively, and a V_{H} CDR1, a V_{H} CDR2, and a V_{H} CDR3 comprising amino acid sequences as set forth in SEQ ID NOS:16, 17, and 18, respectively.

In some embodiments of various methods provided herein, the anti-LAG3 antibody or antigen binding fragment thereof comprises a V_{L} region comprising an amino acid sequence as set forth in SEQ ID NO:14, and a V_{H} region comprising an amino acid sequence as set forth in SEQ ID NO:19.

In other embodiments of various methods provided herein, the anti-LAG3 antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:20.

In certain embodiments of various methods provided herein, the anti-LAG3 antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 41, and 18, respectively.

In certain embodiments of various methods provided herein, the anti-LAG3 antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS: 16, 42, and 18, respectively.

In certain embodiments of various methods provided herein, the anti-LAG3 antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 43, and 18, respectively.

In certain embodiments of various methods provided herein, the anti-LAG3 antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 44, and 18, respectively.

In some embodiments of various methods provided herein, the anti-LAG3 antibody or antigen binding fragment thereof comprises a V_{L} region comprising an amino acid sequence as set forth in SEQ ID NO:14, and a V_{H} region comprising an amino acid sequence as set forth in SEQ ID NO:45.

In some embodiments of various methods provided herein, the anti-LAG3 antibody or antigen binding fragment thereof comprises a V_{L} region comprising an amino acid sequence as set forth in SEQ ID NO:14, and a V_{H} region comprising an amino acid sequence as set forth in SEQ ID NO:46.

In some embodiments of various methods provided herein, the anti-LAG3 antibody or antigen binding fragment thereof comprises a V_{L} region comprising an amino acid sequence as set forth in SEQ ID NO:14, and a V_{H} region comprising an amino acid sequence as set forth in SEQ ID NO:47.

In some embodiments of various methods provided herein, the anti-LAG3 antibody or antigen binding fragment thereof comprises a V_{L} region comprising an amino acid sequence as set forth in SEQ ID NO:14, and a V_{H} region comprising an amino acid sequence as set forth in SEQ ID NO:48.

In other embodiments of various methods provided herein, the anti-LAG3 antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:49.

In other embodiments of various methods provided herein, the anti-LAG3 antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:50.

In other embodiments of various methods provided herein, the anti-LAG3 antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:51.

In other embodiments of various methods provided herein, the anti-LAG3 antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:52.

In other embodiments of various methods provided herein, the anti-LAG3 antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:53.

In other embodiments of various methods provided herein, the anti-LAG3 antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:54.

In other embodiments of various methods provided herein, the anti-LAG3 antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:55.

In other embodiments of various methods provided herein, the anti-LAG3 antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:56.

In various embodiments, the anti-LAG3 antibody or antigen binding fragment thereof comprises a variant of the amino acid sequences disclosed herein. A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the anti-LAG3 antibody or antigen binding fragment thereof has a V_{L} domain with at least 95%, 90%, 85%, 80%, 75% or 50% sequence homology to one of the V_{L} domains described herein, and exhibits specific binding to LAG3. In another embodiment, the anti-LAG3 antibody or antigen binding fragment thereof has a V_{H} domain with at least 95%, 90%, 85%, 80%, 75% or 50% sequence homology to one of the V_{H} domains described herein, and exhibits specific binding to LAG3. In yet another embodiment, the anti-LAG3 antibody or antigen binding fragment thereof has a V_{L} domain with at least 95%, 90%, 85%, 80%, 75% or 50% sequence homology to one of the V_{L} domains described herein and a V_{H} domain with at least 95%, 90%, 85%, 80%, 75% or 50% sequence homology to one of the V_{H} domains described herein, and exhibits specific binding to LAG3.

In one embodiment, the anti-LAG3 antibody or antigen binding fragment thereof has a V_{L} domain having up to 1, 2, 3, 4, 5 or more amino acid substitutions, deletions and/or additions in one of the V_{L} domains described herein, and exhibits specific binding to LAG3. In another embodiment, the anti-LAG3 antibody or antigen binding fragment thereof has a V_{H} domain having up to 1, 2, 3, 4, 5 or more amino acid substitutions, deletions, and/or additions in one of the V_{H} domains described herein, and exhibits specific binding to LAG3. In yet another embodiment, the anti-LAG3 antibody or antigen binding fragment thereof has a V_{L} domain having up to 1, 2, 3, 4, 5 or more amino acid substitutions, deletions, and/or additions in one of the V_{L} domains described herein and a V_{H} domain having up to 1, 2, 3, 4, 5 or more amino acid substitutions, deletions, and/or additions in one of the V_{H} domains described herein, and exhibits specific binding to LAG3.

In various embodiments, the anti-LAG3 antibody or antigen binding fragment thereof is selected from any class of immunoglobulins, including IgM, IgG, IgD, IgA, and IgE Preferably, the antibody is an IgG antibody. Any isotype of IgG can be used, including IgG₁, IgG₂, IgG₃, and IgG₄. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG antibodies provide for long half-life and for effector functions, such as complement activation and antibody-dependent cellular cytotoxicity, such activities may not be desirable for all uses of the antibody. In such instances, an IgG4 constant domain, for example, may be used. In various embodiments, the heavy chain constant domain contains one or more amino acid mutations (e.g., IgG4 with S228P mutation) to generate desired characteristics of the antibody. These desired characteristics include but are not limited to modified effector functions, physical or chemical stability, half-life of antibody, etc.

Ordinarily, amino acid sequence variants of the anti-LAG3 antibodies and antigen binding fragments thereof disclosed herein will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., el al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O, et al., “A model of evolutionary change in proteins.” in Atlas of Protein Sequence and Structure, (1978) vol. 5, suppl. 3. M. O. Dayhoff (ed.), pp. 345-352, Natl. Biomed. Res Found., Washington, DC; Schwartz, R. M., et al., “Matrices for detecting distant relationships.” in Atlas of Protein Sequence and Structure, (1978) vol. 5, suppl. 3. M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., el al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

In some embodiments, the anti-LAG3 antibody is a human antibody. In other embodiments, the anti-LAG3 antibody is a humanized antibody.

In some embodiments, the heavy chain of the anti-LAG3 antibody has a human IgG1 backbone. In other embodiments, the heavy chain of the anti-LAG3 antibody has a human IgG2 backbone. In yet other embodiments, the heavy chain of the anti-LAG3 antibody has a human IgG3 backbone. In still other embodiments, the heavy chain of the anti-LAG3 antibody has a human IgG4 backbone.

In some embodiments, the heavy chain of the anti-LAG3 antibody has a human IgG1 variant backbone. In other embodiments, the heavy chain of the anti-LAG3 antibody has a human IgG2 variant backbone. In yet other embodiments, the heavy chain of the anti-LAG3 antibody has a human IgG3 variant backbone. In still other embodiments, the heavy chain of the anti-LAG3 antibody has a human IgG4 variant (e.g., IgG4 with S228P mutation) backbone.

In certain embodiments, the anti-LAG3 antibody is selected from the group consisting of BMS-986016, REGN3767. LAG525, and GSK2813781. In one embodiment, the anti-LAG3 antibody is BMS-986016. In another embodiment, the anti-LAG3 antibody is REGN3767. In yet another embodiment, the anti-LAG3 antibody is LAG525. In still another embodiment, the anti-LAG3 antibody is GSK2813781.

In some embodiments, the anti-LAG3 antibody can be any antibody, antigen binding fragment thereof, or variant thereof disclosed in WO 2016/028672, the disclosure of which is incorporated by reference herein in its entirety.

### 5. Anti-ILT4 Antibodies and Antigen Binding Fragments Thereof

Provided herein are anti-ILT4 antibodies or antigen binding fragments thereof that can be used in the various methods disclosed herein. Any antibodies that bind to any chemical compound or biological molecule that blocks binding of ILT4 to HLA-G, HLA-A, HLA-B, HLA-F, and/or ANGPTL (such as ANGPTL1, ANGPTL4, or ANGPTL7). Any antibodies that bind to an ILT4 polypeptide, an ILT4 polypeptide fragment, an ILT4 peptide, or an ILT4 epitope and block the interaction between ILT4 and HLA-G, HLA-A, HLA-B, HLA-F, and/or ANGPTL (such as ANGPTL1, ANGPTL4, or ANGPTL7) can be used.

In certain embodiments of various methods, kits, or uses provided herein, the anti-ILT4 antibody or antigen binding fragment thereof comprises a V_{L} CDR1, a V_{L} CDR2, and a V_{L} CDR3 comprising amino acid sequences as set forth in SEQ ID NOs:105, 106, and 107, respectively, and a V_{H} CDR1, a V_{H} CDR2, and a V_{H} CDR3 comprising amino acid sequences as set forth in SEQ ID NOs 10, 111, and 112, respectively.

In some embodiments of various methods, kits, or uses provided herein, the anti-ILT4 antibody or antigen binding fragment thereof comprises a V_{L} region comprising an amino acid sequence as set forth in SEQ ID NO:108, and a V_{H} region comprising an amino acid sequence as set forth in SEQ ID NO: 113.

In other embodiments of various methods, kits, or uses provided herein, the anti-ILT4 antibody or antigen binding fragment thereof comprises a light chain comprising or consisting of an amino acid sequence as set forth in SEQ ID NO:109 and a heavy chain comprising or consisting of an amino acid sequence as set forth in SEQ ID NO:114.

In some embodiments, the anti-ILT4 antibody can be any antibody, antigen binding fragment thereof, or variant thereof disclosed in WO 2018/187518 and WO 2019/126514, the disclosures of which are incorporated by reference herein in their entireties.

In various embodiments, the anti-ILT4 antibody or antigen binding fragment thereof comprises a variant of the amino acid sequences of the anti-ILT4

antibodies disclosed herein. A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the anti-ILT4 antibody or antigen binding fragment thereof has a V_{L} domain with at least 95%, 90%, 85%, 80%, 75% or 50% sequence homology to one of the V_{L} domains of the anti-ILT4 antibodies described herein, and exhibits specific binding to ILT4. In another embodiment, the anti-ILT4 antibody or antigen binding fragment thereof has a V_{H} domain with at least 95%, 90%, 85%, 80%, 75% or 50% sequence homology to one of the V_{H} domains of the anti-ILT4 antibodies described herein, and exhibits specific binding to ILT4. In yet another embodiment, the anti-ILT4 antibody or antigen binding fragment thereof has a V_{L} domain with at least 95%, 90%, 85%, 80%, 75% or 50% sequence homology to one of the V_{L} domains of the anti-ILT4 antibodies described herein and a Vi domain with at least 95%, 90%, 85%, 80%, 75% or 50% sequence homology to one of the V_{H} domains of the anti-ILT4 antibodies described herein, and exhibits specific binding to ILT4.

In one embodiment, the anti-ILT4 antibody or antigen binding fragment thereof has a V_{L} domain having up to 1, 2, 3, 4, 5 or more amino acid substitutions, deletions and/or additions in one of the V_{L} domains of the anti-ILT4 antibodies described herein, and exhibits specific binding to ILT4. In another embodiment, the anti-ILT4 antibody or antigen binding fragment thereof has a V_{H} domain having up to 1, 2, 3, 4, 5 or more amino acid substitutions, deletions, and/or additions in one of the V_{H} domains of the anti-ILT4 antibodies described herein, and exhibits specific binding to ILT4. In yet another embodiment, the anti-ILT4 antibody or antigen binding fragment thereof has a V_{L} domain having up to 1, 2, 3, 4, 5 or more amino acid substitutions, deletions, and/or additions in one of the V_{L} domains of the anti-ILT4 antibodies described herein and a V_{H} domain having up to 1, 2, 3, 4, 5 or more amino acid substitutions, deletions, and/or additions in one of the V_{H} domains of the anti-ILT4 antibodies described herein, and exhibits specific binding to ILT4.

In various embodiments, the anti-ILT4 antibody or antigen binding fragment thereof is selected from any class of immunoglobulins, including IgM, IgG, IgD, IgA, and IgE. Preferably, the antibody is an IgG antibody. Any isotype of IgG can be used,

including IgG₁, IgG₂, IgG₃, and IgG₄. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances, an IgG4 constant domain, for example, may be used. In various embodiments, the heavy chain constant domain contains one or more amino acid mutations (e.g., IgG4 with S228P mutation) to generate desired characteristics of the antibody. These desired characteristics include but are not limited to modified effector functions, physical or chemical stability, half-life of antibody, etc.

Ordinarily, amino acid sequence variants of the anti-ILT4 antibodies and antigen binding fragments thereof disclosed herein will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215.403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary

change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3. M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

In some embodiments, the anti-ILT4 antibody is a human antibody. In other embodiments, the anti-ILT4 antibody is a humanized antibody.

In some embodiments, the light chain of the anti-ILT4 antibody has a human kappa backbone. In other embodiments, the light chain of the anti-ILT4 antibody has a human lambda backbone.

In some embodiments, the heavy chain of the anti-ILT4 antibody has a human IgG1 backbone. In other embodiments, the heavy chain of the anti-ILT4 antibody has a human IgG2 backbone. In yet other embodiments, the heavy chain of the anti-ILT4 antibody has a human IgG3 backbone. In still other embodiments, the heavy chain of the anti-ILT4 antibody has a human IgG4 backbone.

In some embodiments, the heavy chain of the anti-ILT4 antibody has a human IgG1 variant backbone. In other embodiments, the heavy chain of the anti-ILT4 antibody has a human IgG2 variant backbone. In yet other embodiments, the heavy chain of the anti-ILT4 antibody has a human IgG3 variant backbone. In still other embodiments, the heavy chain of the anti-ILT4 antibody has a human IgG4 variant (e.g., IgG4 with S228P mutation) backbone.

In certain embodiments, the ILT4 antagonist is a molecule that binds to HLA-G, HLA-A, HLA-B, HLA-F, ANGPTL1, ANGPTL4, or ANGPTL7 and blocks the binding of ILT4 to HLA-G, HLA-A, HLA-B, HLA-F, ANGPTL1, ANGPTL4, or ANGPTL7. In one

embodiment, the ILT4 antagonist is a molecule that binds to HLA-G and blocks the binding of ILT4 to HLA-G. In one embodiment, the ILT4 antagonist is a molecule that binds to HLA-A and blocks the binding of ILT4 to HLA-A. In one embodiment, the ILT4 antagonist is a molecule that binds to HLA-B and blocks the binding of ILT4 to HLA-B. In one embodiment, the ILT4 antagonist is a molecule that binds to HLA-F and blocks the binding of ILT4 to HLA-F. In one embodiment, the ILT4 antagonist is a molecule that binds to ANGPTL1 and blocks the binding of ILT4 to ANGPTL1. In one embodiment, the ILT4 antagonist is a molecule that binds to ANGPTL4 and blocks the binding of ILT4 to ANGPTL4. In one embodiment, the ILT4 antagonist is a molecule that binds to ANGPTL7 and blocks the binding of ILT4 to ANGPTL7. In some embodiments, the molecule that binds to HLA-G, HLA-A, HLA-B, HLA-F, ANGPTL1, ANGPTL4, or ANGPTL7 is an antibody specifically binding to HLA-G, HLA-A, HLA-B, HLA-F, ANGPTL1, ANGPTL4, or ANGPTL7.

### 6. Analysis of Critical Quality Attributes

In some embodiments, provided are methods for determining including assessing, analyzing, detecting, monitoring and/or quantifying one or more critical quality attributes of a co-formulation that comprises two or more different types of antibodies or antigen binding fragments thereof. In some embodiments, the method comprises (i) preparing a sample of the co-formulation; and (ii) performing an analytical method on the sample to measure the critical quality attribute of each of the two or more different types of antibodies or antigen binding fragments simultaneously.

In some embodiments, the analytical method excludes hydrophobic interaction chromatography (HIC) and reverse phase liquid chromatography (RPLC). While both HIC and RPLC have potential for monitoring antibody oxidation, in some embodiments, HIC or RPLC may not provide sufficient separation of oxidized molecules in a co-formulation.

In some embodiments, the analytical method is a liquid chromatography-mass spectrometry (LC-MS) technique. LC-MS methods based on the complex MS instruments may provide high selectivity and/or sensitivity to analyze PTMs including the oxidation of peptides. However, their cost, extensive tuning requirements, and need for highly trained operators limit their use in non-specialist laboratories. This challenge is magnified when considering use in the QC or good manufacturing practice (GMP) environment. The present disclosure recognizes that the high selectivity and sensitivity of MS-based detection is highly desired in the QC or GMP.

### Sample Preparation

In some embodiments, the step of preparing the sample comprises digesting the two or more different types of antibodies or antigen binding fragments thereof by mixing a protease with the sample. In some embodiments, the protease is selected from the group consisting of Arg-C, Asp-N, chymotrypsin, elastase, endo H, Glu-C, IdeS Protease, IdeZ Protease, Lys-C, Lys-N, pepsin, PNGase F, rAsp-N, rLys-C, thermolysin, trypsin, and combinations thereof.

In some embodiments, the protease is mixed with the sample for about 45 mins to 90 mins. In some embodiments, the protease is mixed with the sample for about 55 mins to 75 mins. In some embodiments, the protease is mixed with the sample for about 60 mins to 70 mins or about 65 mins. In some embodiments, the protease is mixed with the sample for about 15 mins to 45 mins. In some embodiments, the protease is mixed with the sample for about 25 mins to 35 mins. In some embodiments, the protease is mixed with the sample for about 30 mins.

In some embodiments, the protease is mixed with the sample at a temperature within a range of about 25° C. to 40° C. In some embodiments, the protease is mixed with the sample at a temperature within a range of about 30° C. to 40° C. In some embodiments, the protease is mixed with the sample at a temperature within a range of about 35° C. to 40° C. In some embodiments, the protease is mixed with the sample at a temperature within a range of about 35° C. to 39° C. In some embodiments, the protease is mixed with the sample at a temperature of about 37° C.

In some embodiments, the protease is mixed with the sample in the presence of shaking (e.g., about 100 revolutions per minute (rpm), about 200 rpm, about 300 rpm, about 400 rpm, or about 500 rpm).

In some embodiments, the protease comprises Lys-C for a co-formulation comprising an anti-PD-1 antibody or antigen binding fragment thereof and an anti-TIGIT antibody or antigen binding fragment thereof. In some embodiments, a concentration of Lys-C in the sample is within a range of about 0.001 to 0.01 g/L, about 0.005 to 0.01 g/L, or about 0.005 to 0.008 g/L. In some embodiments, the protease is mixed with the sample for about 45 mins to 90 mins, about 55 mins to 75 mins, about 60 mins to 70 mins or about 65 mins. In some embodiments, the protease is mixed with the sample at a temperature within a range of about 25° C. to 40° C., about 30° C. to 40° C., about 35° C. to 40° C., or about 35° C. to 39° C. In some embodiments, the protease is mixed with the sample at a temperature of about 37° C.

In some embodiments, trypsin may result in incomplete digestion, missed cleavage, and/or lower solubility and stability of tryptic peptides for the co-formulation comprising the anti-PD-1 antibody or antigen binding fragment thereof and the anti-TIGIT antibody or antigen binding fragment thereof.

In some embodiments, the protease comprises trypsin for the co-formulation comprising the anti-PD-1 antibody or antigen binding fragment thereof and the anti-LAG3 antibody or antigen binding fragment thereof. In some embodiments, a concentration of trypsin in the sample is within a range of about 0.001 to 0.1 g/L, about 0.005 to 0.05 g/L, or about 0.008 to 0.02 g/L. In some embodiments, the protease is mixed with the sample for about 15 mins to 45 mins, about 25 mins to 35 mins, or about 30 mins. In some embodiments, the protease is mixed with the sample at a temperature within a range of about 25° C. to 40° C., about 30° C. to 40° C., about 35° C. to 40° C., or about 35° C. to 39° C. In some embodiments, the protease is mixed with the sample at a temperature of about 37° C.

In some embodiments, the protease comprises Lys-C for the co-formulation comprising the anti-PD-1 antibody or antigen binding fragment thereof and the anti-ILT4 antibody or antigen binding fragment thereof. In some embodiments, a concentration of Lys-C is within a range of about 0.001 to 0.01 g/L, about 0.005 to 0.01 g/L, or about 0.005 to 0.008 g/L. In some embodiments, the protease is mixed with the sample for about 45 mins to 90 mins, about 55 mins to 75 mins, about 60 mins to 70 mins, or about 65 mins. In some embodiments, the protease is mixed with the sample at a temperature within a range of about 25° C. to 40° C., about 30° C. to 40° C., about 35° C. to 40° C., or about 35° C. to 39° C. In some embodiments, the protease is mixed with the sample at a temperature of about 37° C.

In some embodiments, trypsin may result in incomplete digestion, missed cleavage, and/or lower solubility and stability of tryptic peptides for the co-formulation comprising the anti-PD-1 antibody or antigen binding fragment thereof and the anti-ILT4 antibody or antigen binding fragment thereof.

In some embodiments, the step of digesting comprises mixing a reducing agent solution with the sample. In some embodiments, the reducing agent solution comprises dithiothreitol.

In some embodiments, the reducing agent solution is mixed with the sample for about 15 mins to 45 mins. In some embodiments, the reducing agent solution is mixed with the sample for about 20 mins to 40 mins. In some embodiments, the reducing agent solution is mixed with the sample for about 25 mins to 35 mins. In some embodiments, the reducing agent solution is mixed with the sample for about 28 mins to 32 mins. In some embodiments, the reducing agent solution is mixed with the sample for about 30 mins. In some embodiments, the reducing agent solution is mixed with the sample for about 5 mins to 30 mins. In some embodiments, the reducing agent solution is mixed with the sample for about 5 mins to 25 mins. In some embodiments, the reducing agent solution is mixed with the sample for about 5 mins to 15 mins. In some embodiments, the reducing agent solution is mixed with the sample for about 8 mins to 12 mins. In some embodiments, the reducing agent solution is mixed with the sample for about 10 mins.

In some embodiments, the reducing agent solution is mixed with the sample at a temperature within a range of about 25° C. to 40° C. In some embodiments, the reducing agent solution is mixed with the sample at a temperature within a range of about 30° C. to 40° C. In some embodiments, the reducing agent solution is mixed with the sample at a temperature within a range of about 35° C. to 40° C. In some embodiments, the reducing agent solution is mixed with the sample at a temperature within a range of about 35° C. to 39° C. In some embodiments, the reducing agent solution is mixed with the sample at a temperature of about 37° C. In some embodiments, the reducing agent solution is mixed with the sample at a temperature within a range of about 50° C. to 100° C. In some embodiments, the reducing agent solution is mixed with the sample at a temperature within a range of about 60° C. to 90° C. In some embodiments, the reducing agent solution is mixed with the sample at a temperature within a range of about 70° C. to 90° C. In some embodiments, the reducing agent solution is mixed with the sample at a temperature within a range of about 72° C. to 88° C. In some embodiments, the reducing agent solution is mixed with the sample at a temperature of about 80° C.

In some embodiments, the reducing agent solution is mixed with the sample for about 15 mins to 45 mins, about 20 mins to 40 mins, about 25 mins to 35 mins, about 28 mins to 32 mins or about 30 mins for the co-formulation comprising the anti-PD-1 antibody or antigen binding fragment thereof and the anti-TIGIT antibody or antigen binding fragment thereof. In some embodiments, the reducing agent solution is mixed with the sample at a temperature of about 25° C. to 40° C., about 30° C. to 40° C., about 35° C. to 40° C., about 35° C. to 39° C., or about 37° C. for the co-formulation comprising the anti-PD-1 antibody or antigen binding fragment thereof and the anti-TIGIT antibody or antigen binding fragment thereof.

In some embodiments, the reducing agent solution is mixed with the sample for about 5 mins to 30 mins, about 5 mins to 25 mins, about 5 mins to 15 mins, about 8 mins to 12 mins, or about 10 mins for the co-formulation comprising the anti-PD-1 antibody or antigen binding fragment thereof and the anti-LAG3 antibody or antigen binding fragment thereof. In some embodiments, the reducing agent solution is mixed with the sample at a temperature of about 50° C. to 100° C., about 60° C. to 90° C., about 70° C. to 90° C., about 72° C. to 88° C., or about 80° C. for the co-formulation comprising the anti-PD-1 antibody or antigen binding fragment thereof and the anti-LAG3 antibody or antigen binding fragment thereof.

In some embodiments, the reducing agent solution is mixed with the sample for about 15 mins to 45 mins, about 20 mins to 40 mins, about 25 mins to 35 mins, about 28 mins to 32 mins, or about 30 mins for the co-formulation comprising the anti-PD-1 antibody or antigen binding fragment thereof and the anti-ILT4 antibody or antigen binding fragment thereof. In some embodiments, the reducing agent solution is mixed with the sample at a temperature of about 25° C. to 40° C., about 30° C. to 40° C., about 35° C. to 40° C., about 35° C. to 39° C., or about 37° C. for the co-formulation comprising the anti-PD-1 antibody or antigen binding fragment thereof and the anti-ILT4 antibody or antigen binding fragment thereof.

In some embodiments, the step of digesting comprises mixing an alkylating agent with the sample. In some embodiments, the alkylating agent comprises iodoacetamide.

In some embodiments, the alkylating agent is mixed with the sample for about 15 mins to 45 mins. In some embodiments, the alkylating agent is mixed with the sample for about 20 mins to 40 mins. In some embodiments, the alkylating agent is mixed with the sample for about 25 mins to 35 mins. In some embodiments, the alkylating agent is mixed with the sample for about 28 mins to 32 mins. In some embodiments, the alkylating agent is mixed with the sample for about 30 mins.

In some embodiments, the alkylating agent is mixed with the sample at a temperature of about 25° C. to 40° C., about 30° C. to 40° C., about 35° C. to 40° C., about 35° C. to 39° C., or about 37° C.

In some embodiments, a concentration of antibodies in an initial solution is about 1 to 10 mg/mL, or about 5 mg/mL for the co-formulation comprising the anti-PD-1 antibody or antigen binding fragment thereof and the anti-TIGIT antibody or antigen binding fragment thereof. In some embodiments, the initial solution is mixed with a reducing agent solution, making a second solution. In some embodiments, the reducing agent solution comprises a denaturing buffer. In some embodiments, a concentration of the reducing agent in the reducing agent solution is about 0.01 to about 0.05 M, or about 0.02 to 0.03M. In some embodiments, a ratio of the initial solution to the reducing agent solution is about 1:10 to 1:1, about 1:3 to 1:5, or about 1:4. In some embodiments, an alkylation solution comprising the alkylating agent is added to the second solution, making a third solution. In some embodiments, a concentration of the alkylating agent in the alkylation solution is about 0.5 to 1.5 M, or about 1M. In some embodiments, a ratio of the second solution to the alkylation solution is about 100:1 to 10:1, about 50:1 to 10:1, or about 20:1. In some embodiments, the third solution is mixed with a protease solution comprising the protease. In some embodiments, a concentration of protease in the protease solution is about 0.007 to 0.01 g/L, or about 0.0072 to 0.0088 g/L. In some embodiments, a ratio of the third solution to the protease solution is about 1:1 to about 1:10, or about 1:5. In some embodiments, the digestion step is terminated by quenching (e.g., mixing with trifluoroacetic acid (TFA)).

In some embodiments, a concentration of antibodies in an initial solution is about 1 to 10 mg/mL, or about 5 mg/mL for the co-formulation comprising the anti-PD-1 antibody or antigen binding fragment thereof and the anti-LAG3 antibody or antigen binding fragment thereof. In some embodiments, the initial solution is mixed with a reducing agent solution, making a second solution. In some embodiments, the reducing agent solution comprises a denaturing buffer. In some embodiments, a concentration of the reducing agent in the reducing agent solution is about 0.001 to about 0.01 M, or about 0.005 to 0.006 M. In some embodiments, a ratio of the initial solution to the reducing agent solution is about 1:50 to 1:1, or about 1:10 to 1:20. In some embodiments, the second solution is mixed with a protease solution comprising the protease. In some embodiments, a concentration of protease in the protease solution is about 0.005 to 0.05 g/L, or about 0.001 g/L. In some embodiments, a ratio of the second solution to the protease solution is about 1:1 to about 1:10, or about 1:8. In some embodiments, the digestion step is terminated by quenching (e.g., mixing with trifluoroacetic acid (TFA)).

In some embodiments, a concentration of antibodies in an initial solution is about 1 to 10 mg/mL, or about 5 mg/mL for the co-formulation comprising the anti-PD-1 antibody or antigen binding fragment thereof and the anti-ILT4 antibody or antigen binding fragment thereof. In some embodiments, the initial solution is mixed with a reducing agent solution, making a second solution. In some embodiments, the reducing agent solution comprises a denaturing buffer. In some embodiments, a concentration of the reducing agent in the reducing agent solution is about 0.01 to about 0.05 M, or about 0.02 to 0.03M. the In some embodiments, a ratio of the initial solution to the reducing agent solution is about 1:10 to 1:1, about 1:3 to 1:5, or about 1:4. In some embodiments, an alkylation solution comprising the alkylating agent is added to the second solution, making a third solution. In some embodiments, a concentration of the alkylating agent in the alkylation solution is about 0.5 to 1.5 M, or about 1M. In some embodiments, a ratio of the second solution to the alkylation solution is about 100:1 to 10:1, about 50:1 to 10:1, or about 20:1. In some embodiments, the third solution is mixed with a protease solution comprising the protease. In some embodiments, a concentration of protease in the protease solution is about 0.007 to 0.01 g/L, or about 0.0072 to 0.0088 g/L. In some embodiments, a ratio of the third solution to the protease solution is about 1:1 to about 1:10, or about 1:5. In some embodiments, the digestion step is terminated by quenching (e.g., mixing with trifluoroacetic acid (TFA)).

### Liquid Chromatography

In some embodiments, the analytical method comprises (i) applying the co-formulation to a chromatography material; and (ii) eluting with a solution comprising a mobile phase A and a mobile phase B.

In some embodiments, the chromatographic column is stainless steel packed with porous silica chemically bonded with diisobutyloctadecylsilan.

In some embodiments, the chromatography is ultra-performance liquid chromatography (UPLC).

In some embodiments, the elution is a gradient elution.

In some embodiments, the mobile phase A comprises formic acid or trifluoracetic acid. In some embodiments, the mobile phase A comprises acetic acid in water. In some embodiments, a concentration of acetic acid in water is within a range of about 0.01% to 1%. In some embodiments, a concentration of acetic acid in water is within a range of about 0.05% to 0.5%. In some embodiments, a concentration of acetic acid in water is about 0.1%.

In some embodiments, the mobile phase B comprises formic acid or trifluoracetic acid. In some embodiments, the mobile phase B comprises acetic acid in acetonitrile. In some embodiments, a concentration of acetic acid in acetonitrile is within a range of about 0.01% to 1%. In some embodiments, a concentration of acetic acid in acetonitrile is within a range of about 0.05% to 0.5%. In some embodiments, a concentration of acetic acid in acetonitrile is about 0.1%.

In some embodiments, the chromatography is conducted at a temperature within a range of about 60° C. to 100° C. In some embodiments, the chromatography is conducted at a temperature within a range of about 70° C. to 90° C. In some embodiments, the chromatography is conducted at a temperature of about 80° C. In some embodiments, the chromatography is conducted at a temperature within a range of about 40° C. to 90° C. In some embodiments, the chromatography is conducted at a temperature within a range of about 50° C. to 80° C. In some embodiments, the chromatography is conducted at a temperature within a range of about 60° C. to 70° C. In some embodiments, the chromatography is conducted at a temperature of about 65° C. In some embodiments, the chromatography is conducted at a temperature of about 40° C. to 80° C. In some embodiments, the chromatography is conducted at a temperature of about 50° C. to 70° C. In some embodiments, the chromatography is conducted at a temperature of about 60° C.

In some embodiments, the chromatography is conducted at a temperature within a range of about 60° C. to 100° C. for the co-formulation comprising the anti-PD-1 antibody or antigen binding fragment thereof and the anti-TIGIT antibody or antigen binding fragment thereof. In some embodiments, the chromatography is conducted at a temperature within a range of about 70° C. to 90° C. In some embodiments, the chromatography is conducted at a temperature of about 80° C.

In some embodiments, the chromatography is conducted at a temperature within a range of about 50° C. to 80° C. for the co-formulation comprising the anti-PD-1 antibody or antigen binding fragment thereof and the anti-LAG3 antibody or antigen binding fragment thereof. In some embodiments, the chromatography is conducted at a temperature within a range of about 45° C. to 85° C. In some embodiments, the chromatography is conducted at a temperature within a range of about 60° C. to 70° C. In some embodiments, the chromatography is conducted at a temperature of about 65° C.

In some embodiments, the chromatography is conducted at a temperature within a range of about 40° C. to 60° C. for the co-formulation comprising the anti-PD-1 antibody or antigen binding fragment thereof and the anti-ILT4 antibody or antigen binding fragment thereof. In some embodiments, the chromatography is conducted at a temperature within a range of about 50° C. to 70° C. In some embodiments, the chromatography is conducted at a temperature of about 60° C.

In some embodiments, an initial ratio of the mobile phase B to the mobile phase A is within a range of about 10% to 20% with a flow rate within a range of 0.1 mL/min to 1 mL/min.

In some embodiments, an initial ratio of the mobile phase B to the mobile phase A is within a range of about 10% to 20% with a flow rate within a range of 0.1 mL/min to 1 mL/min for the co-formulation comprising the anti-PD-1 antibody or antigen binding fragment thereof and the anti-TIGIT antibody or antigen binding fragment thereof. In some embodiments, an initial ratio of the mobile phase B to the mobile phase A is within a range of about 14% to 18% with a flow rate within a range of about 0.1 mL/min to 0.5 mL/min. In some embodiments, a ratio of the mobile phase B to the mobile phase A is about 16% with a flow rate of about 0.3 mL/min for an initial period. In some embodiments, the initial period is about 1 min to 5 mins, or about 2 mins. In some embodiments, a ratio of the mobile phase B to the mobile phase A is increased after the initial period. In some embodiments, a ratio of the mobile phase B to the mobile phase A is increased to about 30%, 31%, 32%, 33%, 34% or 35% with a flow rate within a range of about 0.1 ml/min to 0.5 mL/min during a second period. In some embodiments, the second period is within a range of about 20 mins to 30 mins. In some embodiments, the second period is about 26 mins. In some embodiments, a ratio of the mobile phase B to the mobile phase A is increased to about 32% with a flow rate within a range of about 0.3 mL/min and the second period is about 26 mins. In some embodiments, a ratio of the mobile phase B to the mobile phase A is increased after the second period. In some embodiments, a ratio of the mobile phase B to the mobile phase A is increased to about 90%, 91%, 92%, 93%, 94%, 95%, 96%, 97%, 98%, or 99% with a flow rate within a range of about 0.1 ml/min to 0.5 mL/min during a third period. In some embodiments, the third period is within a range of about 1 min to 5 mins. In some embodiments, the third period is about 3 mins. In some embodiments, a ratio of the mobile phase B to the mobile phase A is increased to about 95% with a flow rate within a range of about 0.3 mL/min and the third period is about 3 mins.

In some embodiments, an initial ratio of the mobile phase B to the mobile phase A is within a range of about 10% to 20% with a flow rate within a range of 0.1 mL/min to 1 mL/min for the co-formulation comprising the anti-PD-1 antibody or antigen binding fragment thereof and the anti-LAG3 antibody or antigen binding fragment thereof. In some embodiments, an initial ratio of the mobile phase B to the mobile phase A is within a range of about 10% to 14% with a flow rate within a range of about 0.3 mL/min to 0.9 mL/min. In some embodiments, a ratio of the mobile phase B to the mobile phase A is about 12% with a flow rate about 0.6 mL/min for an initial period. In some embodiments, a ratio of the mobile phase B to the mobile phase A is increased after the initial period. In some embodiments, the initial period is about 1 min to 5 mins, or about 2 mins. In some embodiments, a ratio of the mobile phase B to the mobile phase A is increased to about 21%, 22%, 23%, 24%, 25%, 26%, 27%, 28%, 29% or 30% with a flow rate within a range of about 0.5 mL/min to 1 mL/min during a second period. In some embodiments, the second period is within a range of about 10 mins to 15 mins. In some embodiments, the second period is about 12 mins. In some embodiments, a ratio of the mobile phase B to the mobile phase A is increased to about 25% with a flow rate within a range of about 0.6 mL/min and the second period is about 12 mins. In some embodiments, a ratio of the mobile phase B to the mobile phase A is increased after the second period. In some embodiments, a ratio of the mobile phase B to the mobile phase A is increased to about 90%, 91%, 92%, 93%, 94%, 95%, 96%, 97%, 98%, or 99% with a flow rate within a range of about 0.5 ml/min to 1 mL/min during a third period. In some embodiments, the third period is within a range of about 0.1 min to 1 min. In some embodiments, the third period is about 0.2 min. In some embodiments, a ratio of the mobile phase B to the mobile phase A is increased to about 95% with a flow rate within a range of about 0.8 mL/min and the third period is about 0.2 min.

In some embodiments, an initial ratio of the mobile phase B to the mobile phase A is within a range of about 10% to 20% with a flow rate within a range of 0.1 mL/min to 1 mL/min for the co-formulation comprising the anti-PD-1 antibody or antigen binding fragment thereof and the anti-ILT4 antibody or antigen binding fragment thereof. In some embodiments, an initial ratio of the mobile phase B to the mobile phase A is within a range of about 12% to 18% with a flow rate within a range of about 0.3 mL/min to 0.6 mL/min. In some embodiments, a ratio of the mobile phase B to the mobile phase A is about 15% with a flow rate of about 0.45 mL/min for an initial period. In some embodiments, the initial period is about 1 min to 5 mins, or about 2 mins. In some embodiments, a ratio of the mobile phase B to the mobile phase A is increased after the initial period. In some embodiments, a ratio of the mobile phase B to the mobile phase A is increased to about 33%, 34%, 35%, 34%, 35%, 36%, 36%, 37%, 38%, 39%, or 40% with a flow rate within a range of about 0.3 ml/min to 0.6 mL/min during a second period. In some embodiments, the second period is within a range of about 15 mins to 20 mins. In some embodiments, the second period is about 18 mins. In some embodiments, a ratio of the mobile phase B to the mobile phase A is increased to about 37% with a flow rate within a range of about 0.45 mL/min and the second period is about 18 mins. In some embodiments, a ratio of the mobile phase B to the mobile phase A is increased after the second period. In some embodiments, a ratio of the mobile phase B to the mobile phase A is increased to about 90%, 91%, 92%, 93%, 94%, 95%, 96%, 97%, 98%, or 99% with a flow rate within a range of about 0.3 ml/min to 0.6 mL/min during a third period. In some embodiments, the third period is within a range of about 0.1 min to 5 mins. In some embodiments, the third period is about 1 min. In some embodiments, a ratio of the mobile phase B to the mobile phase A is increased to about 95% with a flow rate within a range of about 0.45 mL/min and the third period is about 1 min.

### Mass Spectroscopy

In some embodiments, electrospray ionization (ESI) is a technique used in mass spectrometry to produce ions using an electrospray in which a high voltage is applied to a liquid to create an aerosol that is ionized. Capillary voltage refers to a voltage applied to the tip of a metal capillary relative to the surrounding source-sampling cone or heated capillary. This strong electric field causes the dispersion of the sample solution into an aerosol of highly charged electrospray droplets.

In some embodiments, an ESI probe is at a temperature within a range of 300° C. to 500° C. In some embodiments, an ESI probe is at a temperature within a range of 350° C. to 450° C. In some embodiments, an ESI probe is at a temperature within a range of 380° C. to 420° C. In some embodiments, an ESI probe is at a temperature within a range of 390° C. to 410° C. In some embodiments, an ESI probe is at a temperature of about 390° C. In some embodiments, an ESI probe is at a temperature of about 400° C. In some embodiments, an ESI probe is at a temperature of about 410° C. In some embodiments, an ESI probe is at a temperature within a range of 400° C. to 700° C. In some embodiments, an ESI probe is at a temperature within a range of 500° C. to 650° C. In some embodiments, an ESI probe is at a temperature within a range of 550° C. to 600° C.

In some embodiments, an ESI probe is at a temperature within a range of 300° C. to 500° C. for the co-formulation comprising the anti-PD-1 antibody or antigen binding fragment thereof and the anti-TIGIT antibody or antigen binding fragment thereof. In some embodiments, an ESI probe is at a temperature within a range of 350° C. to 450° C. In some embodiments, an ESI probe is at a temperature within a range of 380° C. to 420° C. In some embodiments, an ESI probe is at a temperature within a range of 390° C. to 410° C. In some embodiments, an ESI probe is at a temperature of about 390° C. In some embodiments, an ESI probe is at a temperature of about 400° C. In some embodiments, an ESI probe is at a temperature of about 410° C.

In some embodiments, an ESI probe is at a temperature within a range of 400° C. to 700° C. for the co-formulation comprising the anti-PD-1 antibody or antigen binding fragment thereof and the anti-LAG3 antibody or antigen binding fragment thereof. In some embodiments, an ESI probe is at a temperature within a range of 500° C. to 650° C. In some embodiments, an ESI probe is at a temperature within a range of 550° C. to 600° C.

In some embodiments, an ESI probe is at a temperature within a range of 300° C. to 500° C. for the co-formulation comprising the anti-PD-1 antibody or antigen binding fragment thereof and the anti-ILT4 antibody or antigen binding fragment thereof. In some embodiments, an ESI probe is at a temperature within a range of 350° C. to 450° C. In some embodiments, an ESI probe is at a temperature within a range of 380° C. to 420° C. In some embodiments, an ESI probe is at a temperature within a range of 390° C. to 410° C. In some embodiments, an ESI probe is at a temperature of about 390° C. In some embodiments, an ESI probe is at a temperature of about 400° C. In some embodiments, an ESI probe is at a temperature of about 410° C.

In some embodiments, a capillary voltage is within a range of about 1 to 2 kV. In some embodiments, a capillary voltage is within a range of about 1.3 to 1.7 kV. In some embodiments, a capillary voltage is within a range of about 1.3 to 1.5 kV. In some embodiments, a capillary voltage is within a range of about 1.4 to 1.5 kV. In some embodiments, a capillary voltage is about 1.4 kV. In some embodiments, a capillary voltage is about 1.5 kV.

In some embodiments, a capillary voltage is within a range of about 1 to 2 kV for the co-formulation comprising the anti-PD-1 antibody or antigen binding fragment thereof and the anti-TIGIT antibody or antigen binding fragment thereof. In some embodiments, a capillary voltage is within a range of about 1.3 to 1.7 kV. In some embodiments, a capillary voltage is within a range of about 1.4 to 1.5 kV. In some embodiments, a capillary voltage is about 1.4 kV. In some embodiments, a capillary voltage is about 1.5 kV.

In some embodiments, a capillary voltage is within a range of about 1 to 2 kV for the co-formulation comprising the anti-PD-1 antibody or antigen binding fragment thereof and the anti-LAG3 antibody or antigen binding fragment thereof. In some embodiments, a capillary voltage is within a range of about 1.3 to 1.7 kV. In some embodiments, a capillary voltage is within a range of about 1.3 to 1.5 kV.

In some embodiments, a capillary voltage is within a range of about 1 to 2 kV for the co-formulation comprising the anti-PD-1 antibody or antigen binding fragment thereof and the anti-ILT4 antibody or antigen binding fragment thereof. In some embodiments, a capillary voltage is within a range of about 1.3 to 1.7 kV. In some embodiments, a capillary voltage is within a range of about 1.4 to 1.5 kV. In some embodiments, a capillary voltage is about 1.4 kV. In some embodiments, a capillary voltage is about 1.5 kV.

### Critical Parameters

The present disclosure recognizes critical parameters of LC-MS to assess a CQA successfully, including a type of a protease, an amount of a protease, alkylation time, an amount of a reducing agent, digestion time, reduction time, column temperature of LC, a flow rate of LC, an initial ratio of mobile phase B to mobile phase A, ESI probe temperature of MS, a capillary voltage of MS, or combinations thereof.

### Critical Quality Attributes

In some embodiments, the critical quality attribute is selected from the group consisting of oxidation, isomerization, deamidation, disulfide bond modification, and glycosylation. In some embodiments, the critical quality attribute is oxidation. In some embodiments, the critical quality attributes of two different antibodies in a co-formulation is different. In some embodiments two different antibodies in a co-formulation have a common critical quality attribute.

In another aspect, provided are methods for determining including assessing, monitoring and/or quantifying a critical quality attribute of a co-formulation comprising an anti-PD-1 antibody and an anti-TIGIT antibody. In some embodiments, the method comprises (i) preparing a sample of the co-formulation, and (ii) performing a liquid chromatography-mass spectrometry (LC-MS) technique on the sample, wherein the LC-MS measures the critical quality attribute. In some embodiments, the critical quality attribute of the anti-PD-1 antibody is an oxidation at an amino acid corresponding to M105 in SEQ ID NO: 8. In some embodiments, the critical quality attribute of the anti-PD-1 antibody is an oxidation at an amino acid corresponding to M105 in SEQ ID NO: 9. In some embodiments, the critical quality attribute of the anti-PD-1 antibody is an oxidation at an amino acid corresponding to M105 in SEQ ID NO: 10. In some embodiments, the critical quality attribute of the anti-TIGIT antibody is an oxidation at an amino acid corresponding to W104 in SEQ ID NO: 28. In some embodiments, the critical quality attribute of the anti-TIGIT antibody is an oxidation at an amino acid corresponding to W104 in SEQ ID NO: 29. In some embodiments, the critical quality attribute of the anti-TIGIT antibody is an oxidation at an amino acid corresponding to W104 in SEQ ID NO: 30.

In another aspect, provided are methods for assessing, monitoring and/or quantifying a critical quality attribute of a co-formulation comprising an anti-PD-1 antibody and an anti-LAG3 antibody. In some embodiments, the method comprises (i) preparing a sample of the co-formulation, and (ii) performing a liquid chromatography-mass spectrometry (LC-MS) technique on the sample, wherein the LC-MS measures the critical quality attribute. In some embodiments, the critical quality attribute of the anti-PD-1 antibody is an oxidation at an amino acid corresponding to M105 in SEQ ID NO: 8. In some embodiments, the critical quality attribute of the anti-PD-1 antibody is an oxidation at an amino acid corresponding to M105 in SEQ ID NO: 9. In some embodiments, the critical quality attribute of the anti-PD-1 antibody is an oxidation at an amino acid corresponding to M105 in SEQ ID NO: 10. In some embodiments, the critical quality attribute of the anti-LAG3 antibody is an oxidation at an amino acid corresponding to W102 in SEQ ID NO: 18. In some embodiments, the critical quality attribute of the anti-LAG3 antibody is an oxidation at an amino acid corresponding to W102 in SEQ ID NO: 19. In some embodiments, the critical quality attribute of the anti-LAG3 antibody is an oxidation at an amino acid corresponding to W102 in SEQ ID NO: 20.

In another aspect, provided are methods for determining including assessing, monitoring and/or quantifying a critical quality attribute of a co-formulation comprising an anti-PD-1 antibody and an anti-ILT4 antibody. In some embodiments, the method comprises (i) preparing a sample of the co-formulation, and (ii) performing a liquid chromatography-mass spectrometry (LC-MS) technique on the sample, wherein the LC-MS measures the critical quality attribute. In some embodiments, the critical quality attribute of the anti-PD-1 antibody is an oxidation at an amino acid corresponding to M105 in SEQ ID NO: 8. In some embodiments, the critical quality attribute of the anti-PD-1 antibody is an oxidation at an amino acid corresponding to M105 in SEQ ID NO: 9. In some embodiments, the critical quality attribute of the anti-PD-1 antibody is an oxidation at an amino acid corresponding to M105 in SEQ ID NO: 10. In some embodiments, the critical quality attribute of the anti-ILT4 antibody is an oxidation at an amino acid corresponding to W102 in SEQ ID NO: 112. In some embodiments, the critical quality attribute of the anti-ILT4 antibody is an oxidation at an amino acid corresponding to W102 in SEQ ID NO: 113. In some embodiments, the critical quality attribute of the anti-ILT4 antibody is an oxidation at an amino acid corresponding to W102 in SEQ ID NO: 114. In some embodiments, the critical quality attribute of the anti-ILT4 antibody is an oxidation at an amino acid corresponding to W7 in SEQ ID NO: 113. In some embodiments, the critical quality attribute of the anti-ILT4 antibody is an oxidation at an amino acid corresponding to W7 in SEQ ID NO: 114.

### EXAMPLES

### Example 1

The present Example describes exemplary methods for assessing critical quality attributes of a co-formulation comprising two different antibodies, i.e., mAb-1 (an antibody having a light chain comprising an amino acid sequence as set forth in SEQ ID NO: 5 and a heavy chain comprising an amino acid sequence as set forth in SEQ ID NO:10) and mAb-2 (an antibody having a light chain comprising an amino acid sequence as set forth in SEQ ID NO:25 and a heavy chain comprising an amino acid sequence as set forth in SEQ ID NO:30).

### Chemicals and Recombinant Proteins

mAb-1 and mAb-2 were produced in Chinese hamster ovary (CHO) cells at Merck & Co., Inc. (Kenilworth, NJ, USA). A co-formulated drug product was made by mixing mAb-1 and mAb-2 at protein concentration ratios of 1:1. Lysyl Endopeptidase (Lys-C) was purchased from FUJIFILM Wako Chemicals U.S.A. (Richmond, VA). Dithiothreitol (DTT, no-weight format), and iodoacetamide (single use) supplied in amber tubes were obtained from Pierce Protein Biology (Thermo Scientific, Rockford, IL). Tris(hydroxymethyl)aminomethane hydrochloride (Tris-HCl) buffer (1 M), guanidine hydrochloride (GuHCl), sequencing grade trifluoroacetic acid (TFA), purified water (LC-MS grade), acetonitrile (LC-MS Grade), 0.1% formic acid in water (LC-MS Grade), and 0.1% formic acid in acetonitrile (LC-MS Grade) were obtained from Fisher Scientific (Waltham, MA). Ethylenediaminetetraacetic Acid (EDTA) (0.5 M) was purchased from Promega (Madison, WI).

Lys-C Digestion of mAbs

Samples were diluted with purified water to a concentration of 5 mg/mL for each antibody in a 1.5 mL polypropylene tube. The diluted sample (containing 100 μg of total protein in 20 μl) was denatured and reduced by mixing with 83 μL of Denature/Reduction Buffer (composed of 750 μL 8 M GuHCl, 50 μL 1 M Tris-HCl, 10 μL 0.5 M EDTA, and 20 μL 1 M DTT) in Thermomixer (Eppendorf, Hamburg, Germany) at 37° C. with 300 rpm shaking for 30 min. Iodoacetamide (5 μL 1 M) was added to initiate alkylation for 30 min under dark. Following the alkylation, 400 μL Digestion Buffer (composed of 500 μL 1 M Tris-HCl 100 μL 0.5 M EDTA 20 μL 1 M DTT 9.38 mL purified water,) and 100 μL of diluted Lys-C enzyme Solution (composed of one vial of Lyophilized Lys-C and 500 μL of Digestion Buffer) were added, and the mix was allowed to incubate at 37° C. with 300 rpm shaking for 65 min. The digestion was quenched with 20% TFA before analysis.

### QDa Based LC-MS Analysis

Analysis was performed with a Waters ACQUITY Ultra Performance Liquid Chromatography (UPLC) H-Class System equipped with an ACQUITY QDa detector (Waters, Milford, MA). QDa mass spectrometer was operated in the positive Selective Ion Recording (SIR) mode set up with specific m/z channels aimed to detect targeted native and oxidized peptides (Table 3). The QDa was operated with the settings of cone voltage of 15 V, probe temperature of 400° C., capillary voltage of 1.5 kV of sampling Rate of 2 points/sec.

A HALO® Peptide ES-C18 Column (2.7 μm, 160 Å, 2.1 mm×150 mm; HALO, Wilmington, DE) was used for chromatography separation. Aqueous formic acid (0.1%) was used as mobile phase A (NIPA), and 0.1% formic acid in acetonitrile as mobile phase B (MPB). Injection volume was 5 μL. Peaks were eluted using the following gradients: 16% MPB for 2 minutes, 16% to 32% MPB in 26 minutes, 32% to 95% MPB in l minute. The column was then washed with 95% MPB for 3 minutes and re-equilibrated with 16% MPB for 8 minutes. Flow rate was 0.3 mL/min. The column temperature was set at 80° C. A diverter valve was used to switch the flow from the column to QDa within 8.00-29.00 min only of the gradients to minimize detector contamination Peak ID of each SIR channel was also confirmed by running the same chromatography conditions with HRMS (Thermo Scientific™ Q Exactive™ HF-X, Thermo Scientific, Waltham, MA), operated in the positive polarity full scan and dd-MS² mode.

### Data Processing, Calculation, and Statistical Analysis

Data were acquired and processed by using custom field automated calculation with Empower software (Waters, Milford, MA). MassLynx V4.1 (Waters, Milford, MA) was used to calculate the theoretical m/z for the peptide of interest. The component peak areas of all SIR channels, including mAb-1 M105 oxidized peptides (+16 Da for methionine sulfoxide), mAb-2 W104 oxidized peptides (+4, +16, and +32 Da for kynurenine, hydroxytryptophan, and N-formylkynurenine, respectively), and the corresponding native non-oxidized M105 and W104 peptides, were determined. Component peak areas from the QDa SIR channels were used to calculate the percent oxidation (% oxidation) by dividing the sum of oxidized component peak areas by the sum of oxidized and non-oxidized (native peptide) component peak areas.

Design of Experiment (DoE) for assessing the impact of sample preparation parameters was generated by using JMP 15.2.0 (JMP Statistical Discovery LLC., Cary, NC). DoE for evaluating the impact of instrument method parameters was generated by using Fusion Method Development™ Software (S-Matrix, Eureka, CA). All statistical analyses were performed using JMP 15.2.0.

### Oxidation of Methionine and Tryptophan Residues by HIC and RPLC

Oxidation of methionine and tryptophan residues are one of degradation pathways for mAbs. Both component mAbs of the co-formulated mixture were prone to oxidation in the heavy CDR3 region at methionine 105 (M105) of mAb-1 and tryptophan oxidation at position number 104 (W104) of mAb-2, particularly when stored at elevated temperatures with subsequent degrees of impairment to antigen binding affinity.

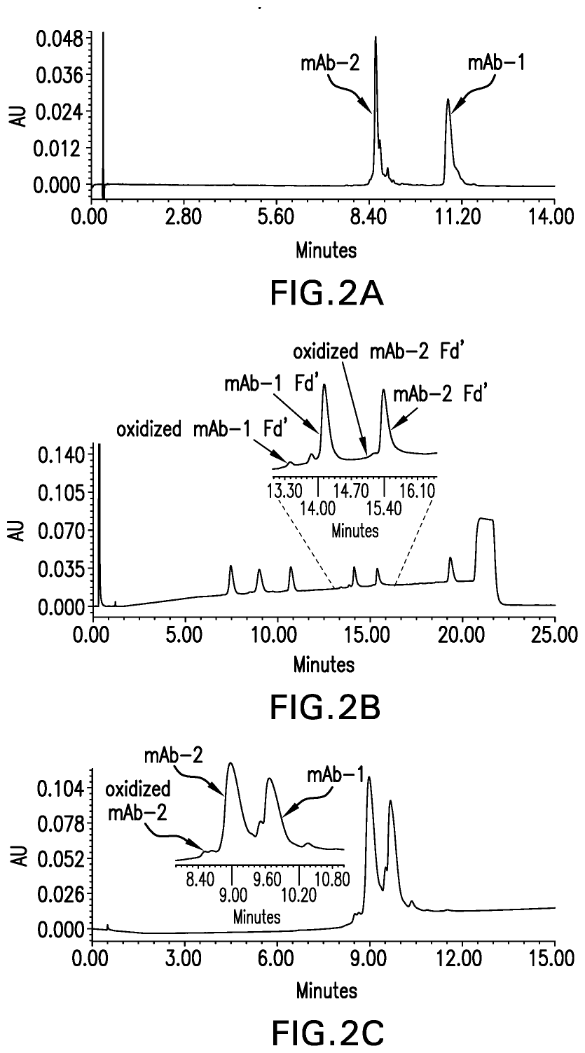

As chromatography-based methods are preferred in commercial QC lab, the feasibility of both HIC and RPLC methods were evaluated. Reversed phase liquid chromatography (RPLC) provided sufficient separation of two intact mAbs. However, the peaks of the oxidized proteins were not well resolved from their corresponding non-oxidized proteins as shown in FIG. 2A. An alternative approach was examined using limited digestion with IdeS Protease followed by disulfide reduction to generate Fd′, light chain (LC), and Fc/2 subunits which were resolved by RPLC. The RPLC separation of the subunits showed an improved resolution between oxidized and non-oxidized proteins as shown in FIG. 2B. However, the large retention time range of the oxidized Fd′ peak of mAb-2 overlapped with the unoxidized Fd′ peak of mAb-1, resulting in a likely overestimated of the oxidation value. This approach was not sufficiently selective for M105 and W104 oxidation, as there were product-related impurities (e.g., protein fragments) coeluted with the oxidized species. Furthermore, the method created challenges in the GMP environment due to low signal intensity and poor separation of the oxidized protein peaks. The separation of intact mAbs was also evaluated by using hydrophilic interaction chromatography (HIC). Significant overlap between the two mAbs as well as limited resolution between oxidized and non-oxidized species were observed as shown in FIG. 2C. In sum, these approaches were not suitable for quantifying M105 and W104 oxidation in the co-formulated drug products.

### Development of a QDa Based Peptide Mapping Method

As both HIC and RPLC failed to provide the sufficient resolution to determine the protein oxidation, a focused peptide mapping method (FPM) with LC-QDa-MS quantitation was developed. Lys-C was used for antibody peptide preparation as it thoroughly digests both mAbs with desired peptide length for QDa analysis. Reduction with DTT provided higher digestion efficiency than with tris(2-carboxyethyl)phosphine (TCEP).

A UPLC gradient elution was developed for separation and detection of targeted mAb-1 M105 and mAb-2 W104 peptides. Formic acid was used in the mobile phases as the benefits of improved signal intensity and precision of low-level oxidized species were more critical than the enhanced chromatographic separation advantages offered by the ion-pairing reagent trifluoroacetic acid.

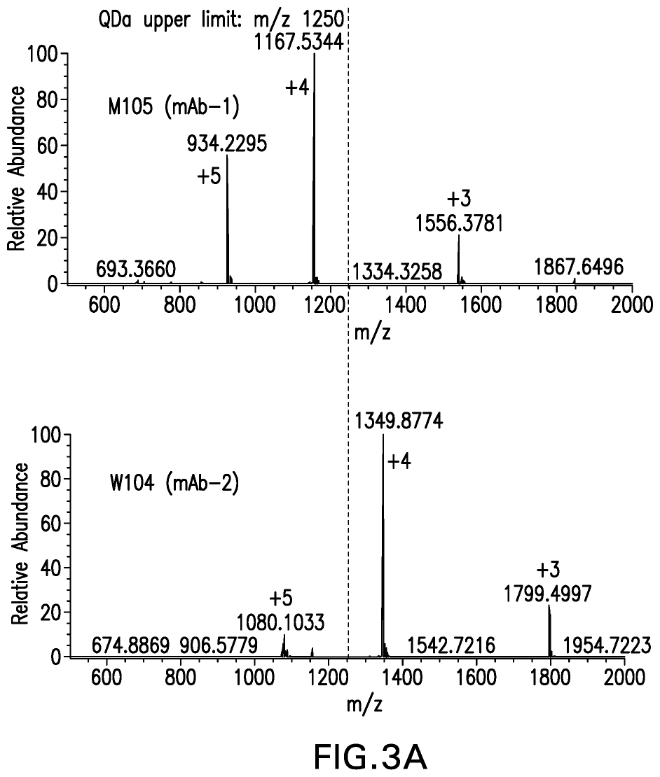

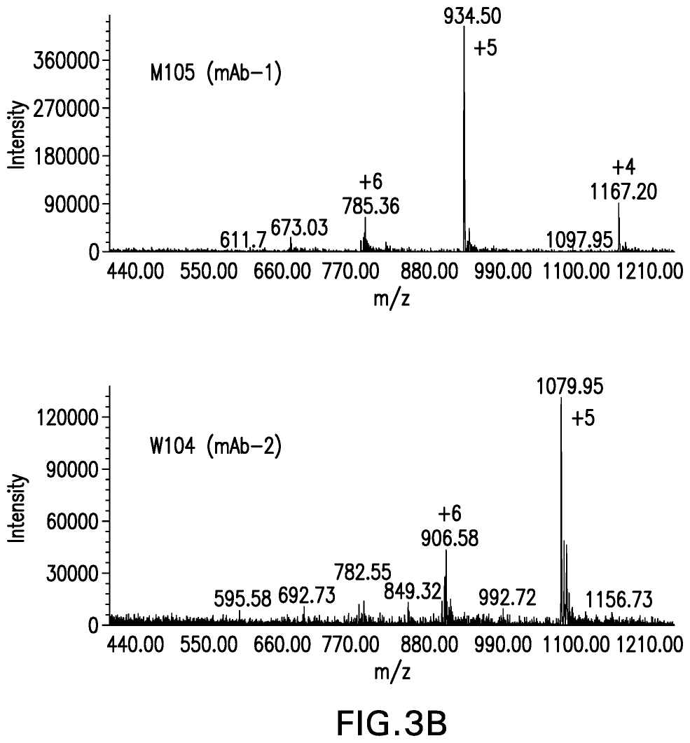

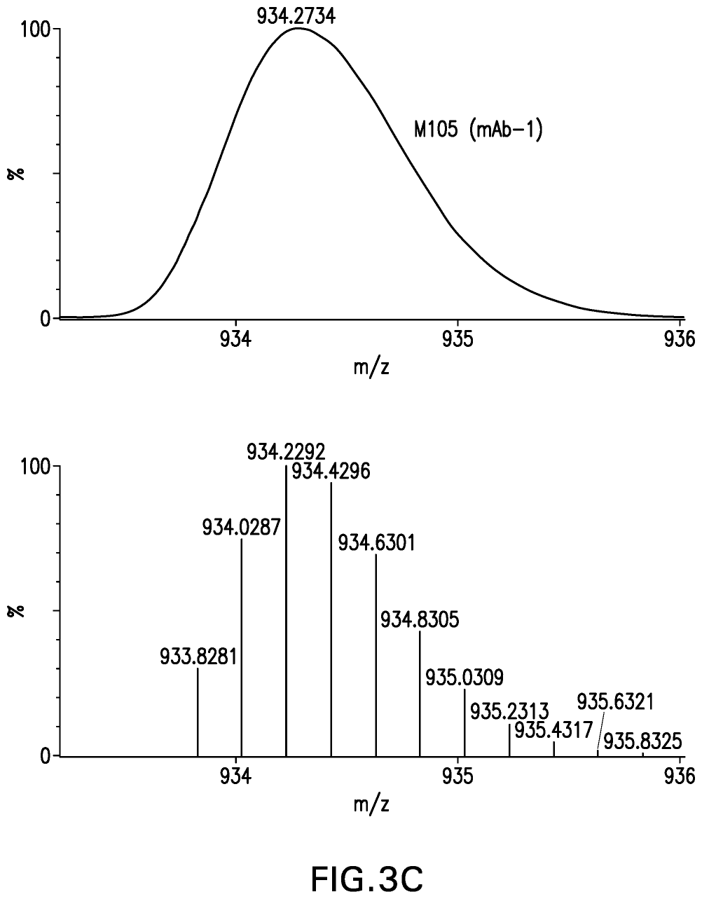

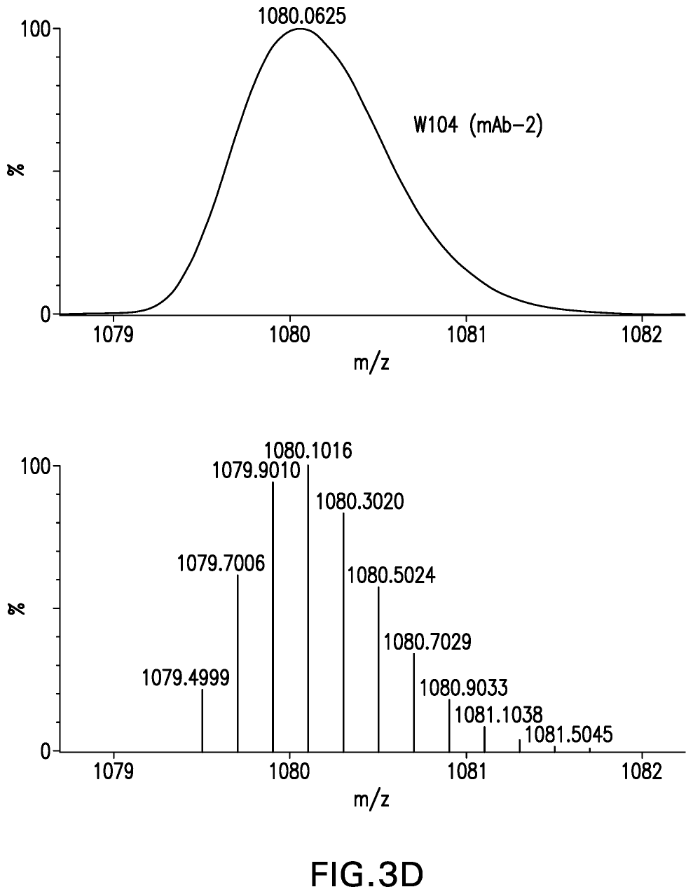

The QDa detector utilized mass spectrometry (MS) for selective detection of the peptides containing the residues of interest (M105/mAb-1, W104/mAb-2) eluted from the column. To set up the selected ion recording (SIR) channels, the charge states of the selected peptides were first collected on a HRMS (FIG. 3A), and then on a QDa mass spectrometer (FIG. 3B).

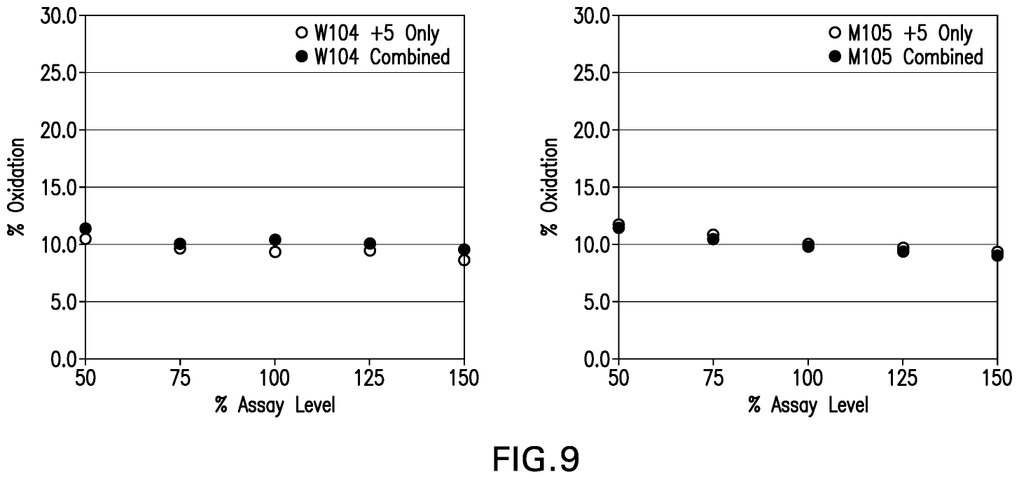

The dominant charge state for both W104 and M105 peptides was +4 by HRMS (Thermo Scientific Q Exactive™), while it was +5 by QDa mass spectrometer. The +3 charge states were not detected by QDa mass spectrometer since the m/z were above the upper limit (1250 Da) of QDa. For quantitation, the +5 charge states of the peptides of interest, rather than the of sum of all charge state peaks, were selected when defining the SIR channels used to calculate % oxidation. The suitability of this choice was verified by comparison with quantitation based on other charge states, which gave similar results as shown in FIG. 9. The theoretical average m/z calculated by MassLynx software (Waters, Milford, MA) was used instead of the monoisotopic mass or the most abundant mass of the peptide of interest (Table 3, FIG. 3C, and FIG. 3D). Using the average mass accommodated the low resolving power of QDa mass spectrometer (0.6 Da) and maximized the coverage of the peptide isotopic envelope.

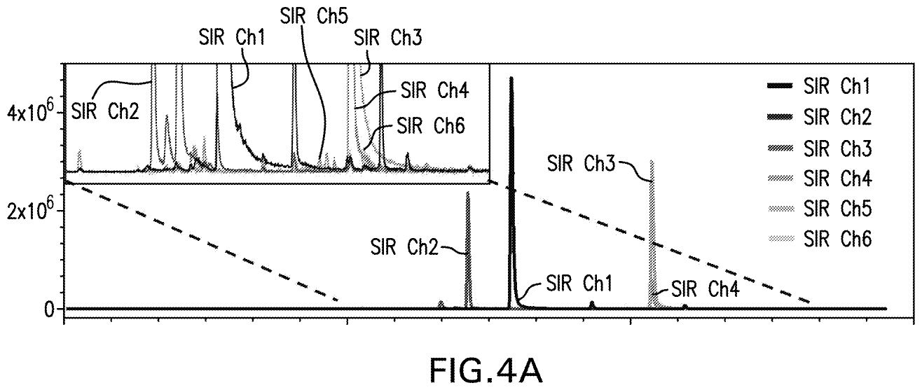

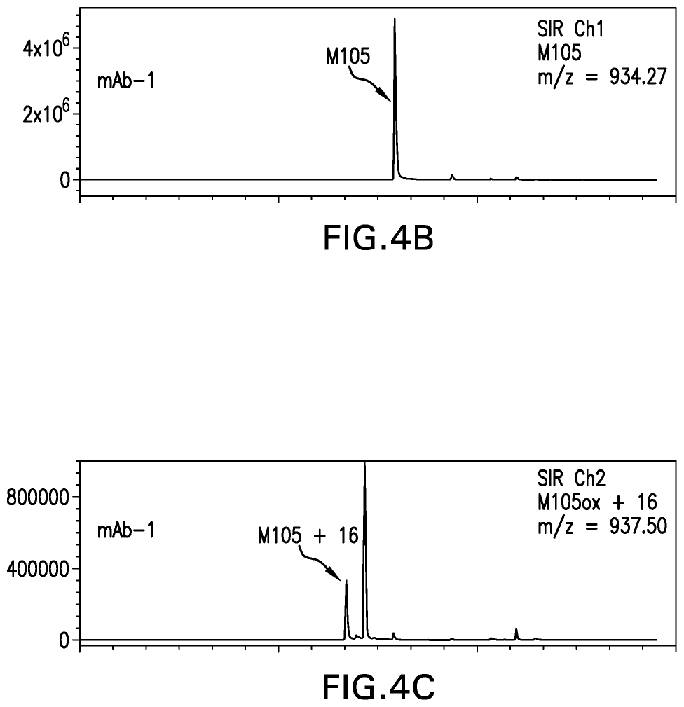

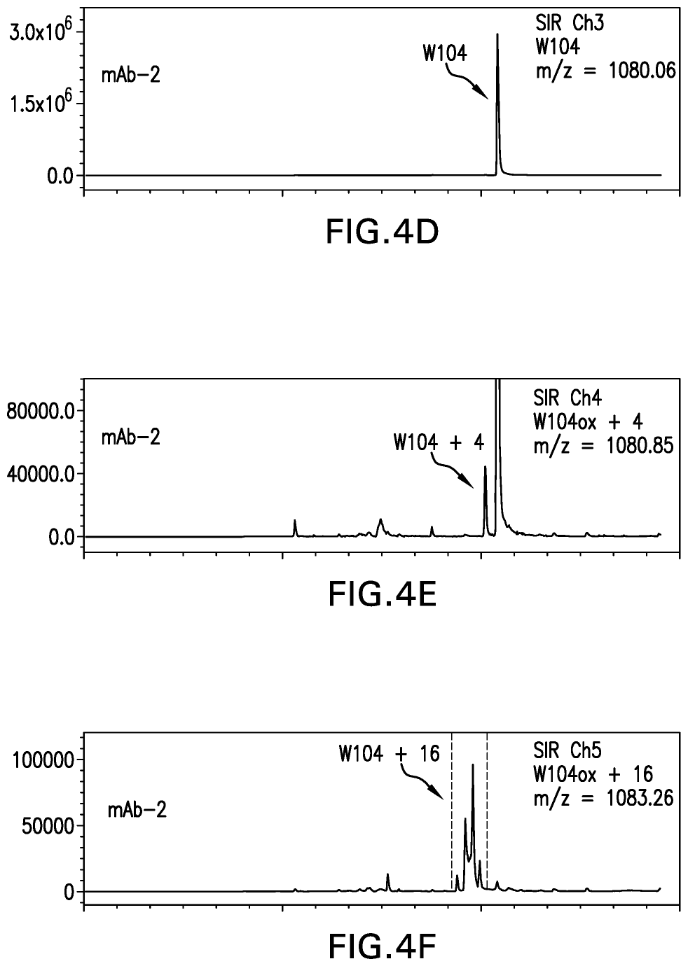

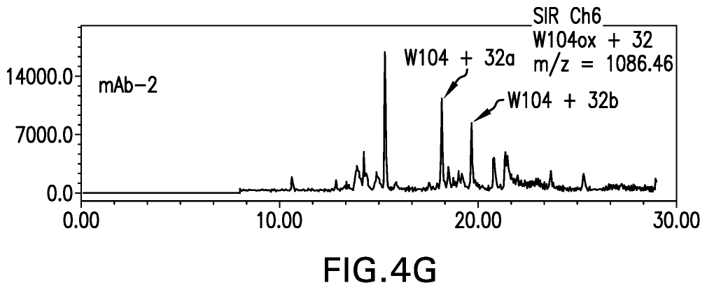

The major oxidized product of M105 was the sulfoxide (+16 Da) compared to the native peptide. In contrast, oxidized W104 peptides contained a mixture of peptides with a mass increase of +4, +16, +32 Da, reflecting the multiple oxidized Trp products typically observed. Six channels were set to detect both non-oxidized and oxidized forms of the relevant peptides, using the +5 charge state of each species. Overlaid and individual SIR channel are shown in FIG. 4. The non-oxidized and oxidized M105 peptides (+16 Da) from mAb-1 were detected by SIR at m/z 934.27 and 937.50 as well resolved chromatographic peaks (FIG. 4B and FIG. 4C). An interfering peak, which was identified by MS/MS as a peptide unrelated to the oxidized M105 peptide, was also detected, due to the limited mass resolution of QDa. The peptide peaks unrelated to oxidation were excluded from calculations to determine % oxidation.

The non-oxidized and oxidized W104 peptides from mAb-2 were detected by SIR at m/z 1080.06, 1080.85, 1083.26, and 1086.46, respectively (FIGS. 4D, 4E, 4F, and 4G). The W104+4 Da oxidized peptide displayed as a single peak, whereas the W104+16 Da appeared as multiple isobaric peaks due to multiple positions for oxidation on the tryptophan aromatic ring. The W104+32 Da peptide contained two isobaric peaks. The identity of all oxidized M105 and W104 peptide peaks in QDa chromatogram was confirmed by HRMS.

### Method Validation

To test the feasibility of implementing this LC-QDa-MS peptide mapping method in the QC and GMP laboratories, a set of validation experiments were performed to establish the accuracy, precision, linearity, range, quantitation limit (QL), specificity, and solution stability. The validation results are summarized in Table 4.

Accuracy and linearity were determined by performing triplicate analyses of sample digests at 30%, 40%, 50%, 75%, 100%, 125% of the target antibody concentration level in the formulation. The % oxidized M105 and % oxidized W104 at each concentration level were compared to those of the 100% target level and were calculated as % recovery (i.e., (determined concentration by method/theoretical concentration)×100%) as shown in Table 4. The recovery ranged from 96-126% for M105, and 94-118% for W104, indicating a good accuracy for the measurement. High-resolution peptide mapping was performed as an alternative method, showing comparable % oxidation values for both non-stressed and stressed DP samples between the two methods. The Stressed DP was exposed to simulated sunlight in a light chamber. It artificially generates the protein oxidation for this method to monitor, since a typical DP doesn't have much protein oxidation. Plotting of the oxidized peptide peak areas vs the concentration levels displayed excellent linearity with the coefficient of determination (R2) of 0.9985 and 0.9883 for M105 and W104 peptides, respectively.

The repeatability (% relative standard deviation, % RSD) was assessed by using two approaches: a) triplicate analyses of peptides from different protein concentration levels and b) 6 independent analyses at the 100% target level. Approach a) showed % RSD (n=3) of 1-3% for mAb-1 M105, and 2-6% for mAb-2 W104 (Table 4). Approach b) showed % RSD (n=6) of 2% for mAb-1 M105, and 9% for mAb-2 W104 (Table 4). Thus LC-QDa MS method demonstrated excellent repeatability (% RSD ≤15%) within the analytical target profile (ATP).

Intermediate precision was assessed using two different analysts, two different UPLC columns, and two different LC-MS instruments. Each analyst conducted six different runs with triplicate sample preparations (n=18) at target concentration level. The % RSD of intermediate precision was calculated for runs (n=18) conducted by both analysts. The % RSD was 9% for mAb-1 M105, and 15% for mAb-2 W104 (Table 4). Absence of interference in specificity was confirmed by analyzing the water blank and placebo that showed no peak ≥QL observed in region of interest for all SIR channels. Ability to detect change in specificity was assessed by analyzing and comparing QDa SIR profiles of unstressed, 0.5x ICH, and 2x ICH light-stressed co-formulated drug product per method.

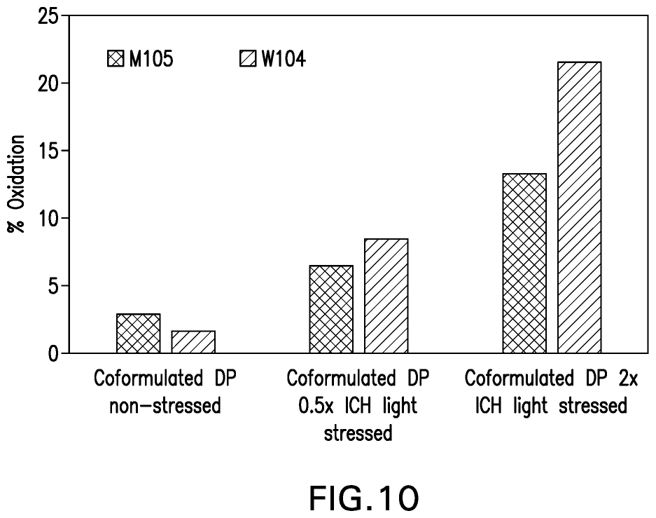

The method was able to monitor simultaneously the changes in methionine and tryptophan oxidation levels in the stressed samples (FIG. 10). Method QL of 0.3% was determined for both M105 and W104 as the lowest acceptable oxidation level in the accuracy and linearity study with criteria of accuracy between 70 to 130% and precision (% RSD) less than 15%. Digested sample solution was determined to be stable at 2-8° C. for at least 3 days with a criterion of % difference from Day 0<15%. A method range of 30-125% was established for both M105 and W104 peptides using the results from accuracy, precision, linearity, and QL (Table 4). Together, these results clearly demonstrated that the QDa based Focused Peptide Mapping (FPM) method was suitable for monitoring oxidation attributes in the co-formulated antibody drug product with acceptable accuracy, precision, linearity, sensitivity, specificity, and solution stability.

### Robustness Study

The robustness study was designed to characterize the method performance over a range of deliberate variations in critical method parameters. FIG. 4 shows the fishbone diagram of the method parameters with critical parameters highlighted. Non-critical or low risk parameters identified by historical experience were not included.

Statistical significance was determined using 95% confidence interval on the changes of the average responses within the studied ranges of the method parameters. Statistical significance, however, did not indicate whether the changes of average responses are large enough to impact the operational ranges of the method parameters. Thus, practical significance based on the acceptable response range (ARR) were established to assess whether the changes of average responses due to the variation of the method parameter are large enough to be meaningful for the robustness assessment of the assay. The acceptance response range for mAb-1 M105 percent oxidation was defined as values within 30% from the average obtained at set point. The ARR for mAb-2 W104 oxidation % was defined as values within 40% from the average value obtained at set point. The ARR was higher for W104 since overall % oxidized W104 is lower than % oxidized M105. The ARR is predefined based on the oxidation value of a typical drug product, which represents the lowest impurity level in the shelf life.

The robustness study consisted of two separate stages (Table 5). Stage 1, which employed a Design of Experiments (DoE) approach, includes DoE-1 and DoE-2. DoE-1 (36 samples) evaluated the critical parameters associated with sample preparation, including DTT volume, reduction time, alkylation time, Lys-C volume, and digestion time. DoE-2 evaluated critical LC instrument method parameters, including MPB % in the eluding solution (at start of gradient), concentration of formic acid in water, concentration of formic acid in acetonitrile, column temperature, and pump flow rate. Both sets of DoE studies in Stage 1 included six additional runs at the method set point to serve as the control for statistical analysis. The Stage 2 study assessed electrospray ionization (ESI) probe temperature, capillary voltage, and Lys-C lots by a one-factor-at-a-time (OFAT) approach while all other method conditions were kept at the set point.

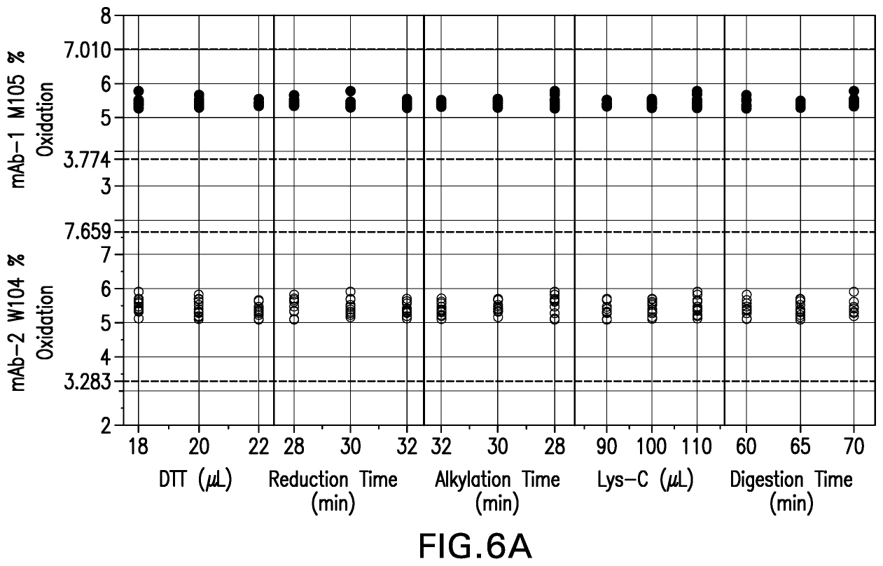

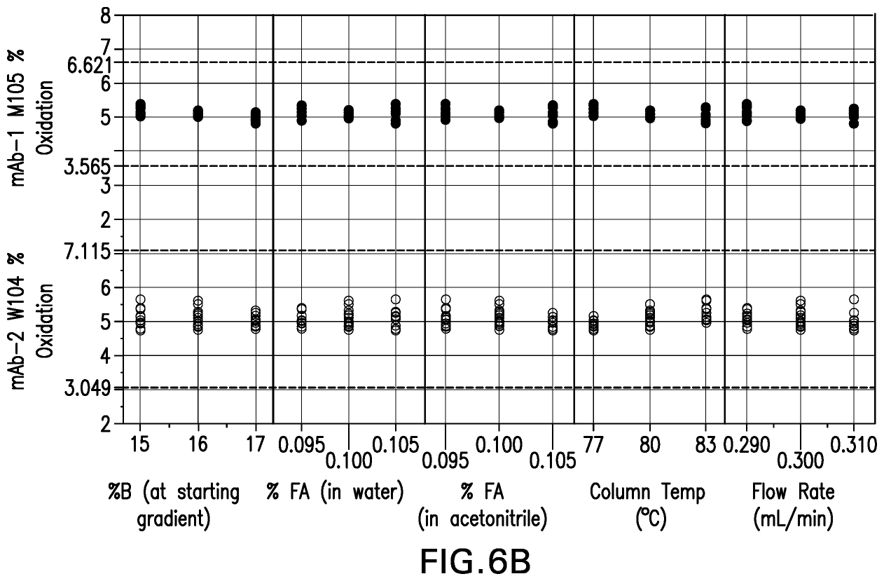

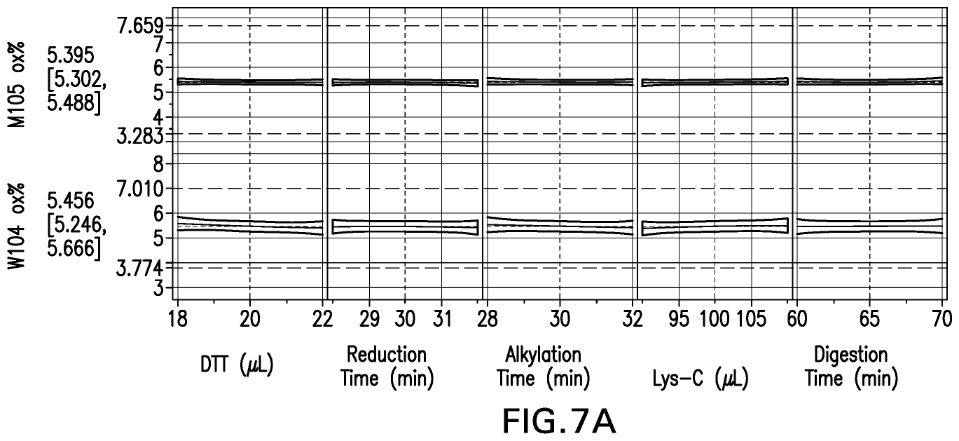

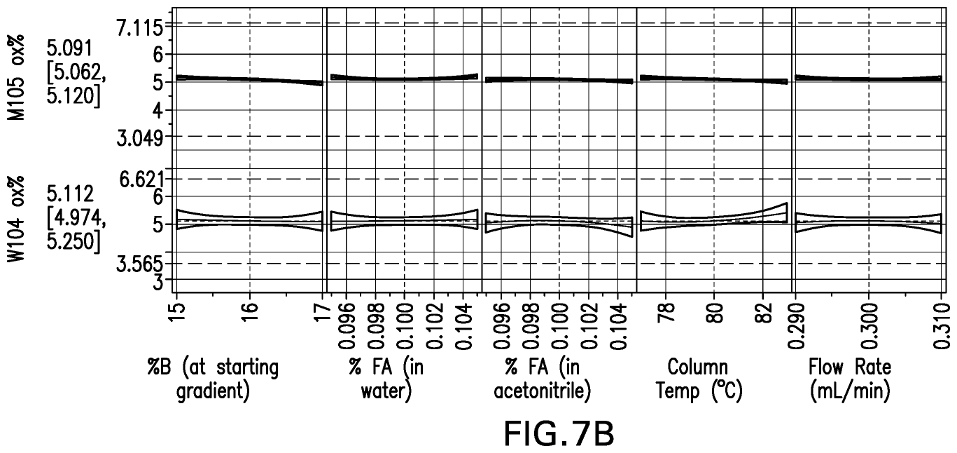

The results of stage 1 DoE-1 for the sample preparation parameters and Stage 1 DoE-2 for the instrument method parameters are summarized in Table 6 and Table 7, respectively. As shown by scatterplots of responses (FIG. 6) and the prediction profiles (FIG. 7), Stage 1 DoE-1 and DoE-2 robustness studies indicated that all measured % oxidation of M105 and W104 peptides fell within their acceptable response ranges with no practical impacts resulting from the deliberate variations of the sample preparation parameters (DoE-1) or instrument method parameters (DoE-2).

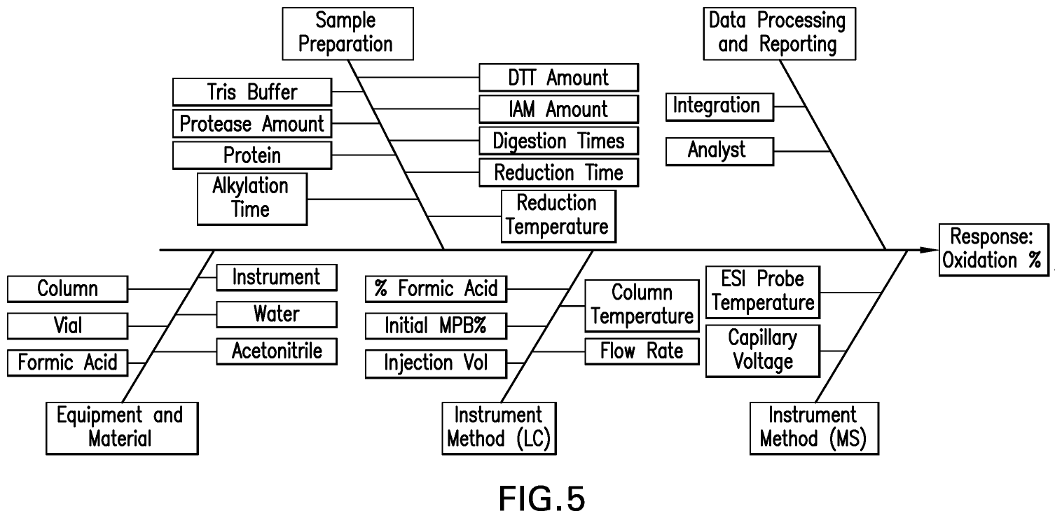

While the method was robust for practical impacts from deliberate variations to critical parameters, there were several instrument method parameters that were deemed to have statistically significant impacts based on the study design. The mAb-2 W104% oxidation measurement was shown to be statistically impacted by column temperature. The mAb-1 M105 oxidation measurement was shown statistically impacted by variation of MPB % (at starting gradient), % formic acid in acetonitrile, column temperature, % formic acid in water, or combined parameters, such as % formic acid in water x % B, % formic acid in water x flow rate, % formic acid in acetonitrile x flow rate, and column temperature x flow rate, on % oxidation. However, all these impacts were within acceptable response range and thus are not considered practically significant for use of this method to determine product quality (FIG. 5 and FIG. 6).

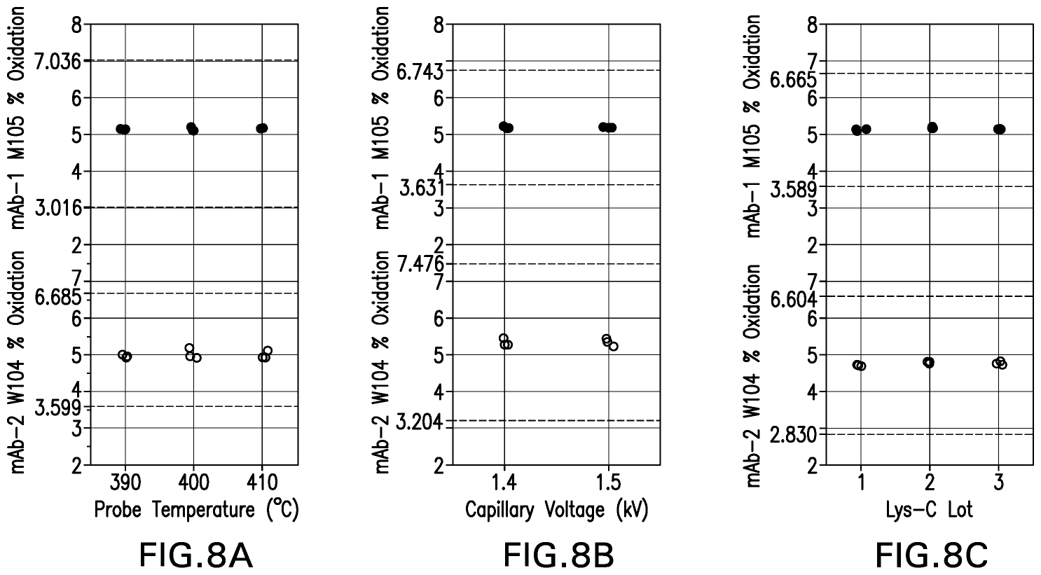

Stage 2 robustness studies involving ESI-MS probe temperature, capillary voltage, and Lys-C lots were performed using Fusion Method Development™ Software using one-factor-at-a-time (OFAT) approach. Triplicate sample preparations were used for each setting of condition. Results indicated all measured % oxidation of M105 and W104 peptides in stage 2 robustness study fell within their acceptable response ranges with no practical impacts (FIG. 8, Tables 8-10).

### CONCLUSION

The oxidized M105 peptides from mAb-1 were well resolved from the native peptide. The more complex set of W104 oxidized peptides from mAb-2, comprised of a mix of +4, +16, and +32 Da peptides, were also well separated to enable their quantitation. The results shows examples of coelution of multiple peptides from the digest, which would interfere with non-selective UV quantitation, presented only minimal impact when the QDa detector is used. Furthermore, the method described therein utilizing the QDa detector underwent successful full method validation per the International Council for Harmonisation of Technical Requirements for Pharmaceuticals for Human Use (ICH) recommendations. In addition, extensive studies were conducted covering the parameters involving both sample preparation and LC instrument parameters to demonstrate that the method is robust, and the variation of the determined % oxidation of M105 and W104 peptides is in a typical acceptable response range in an analytical lab leading to no practical impacts. Implementation of our method in QC or GMP environment to monitor the most critical oxidation site in each antibody in the coformulation with one analysis provided an excellent example for usage of compact mass spectrometer for targeted species in both development and QC laboratories. The advantages of a robust, high throughput, cost effective method that can be executed by non-MS experts will allow this method and others like it to gain increased application in the future biopharmaceutical analytics.

TABLE 3
Channel name, peptide nomenclature,
and m/z monitored for each mAb

| mAb | Channel Name | Peptide | m/z monitored |
|---|---|---|---|
| mAb-1 | SIR Ch1 | M105 | 934.27 |
| mAb-1 | SIR Ch2 | M105ox_16 | 937.50 |
| mAb-2 | SIR Ch3 | W104 | 1080.06 |
| mAb-2 | SIR Ch4 | W104ox_4 | 1080.85 |
| mAb-2 | SIR Ch5 | W104ox_16 | 1083.26 |
| mAb-2 | SIR Ch6 | W104ox_32 | 1086.46 |

TABLE 4
Accuracy, precision, linearity, range, quantitation limit
(QL), and specificity of the QDa based FPM method

| | Accuracy | | | Precision | | | | |
|---|---|---|---|---|---|---|---|---|
| | Assay | % | | ATP | | | Intermediate | |
| Peptide | Level | Recovery | % RSD | (% | Repeatability | ATP | Precision | ATP |
| (Molecule) | (%) | (n = 3) | (n = 3) | Recovery) | (% RSD) | (% RSD) | (% RSD) | (% RSD) |
| M105 | 30 | 100 | 2 | 70-130% | 2% | ≤15% | 9% | ≤15% |
| (mAb-1) | 40 | 103 | 2 | | | | | |
| | 50 | 101 | 1 | | | | | |
| | 75 | 96 | 1 | | | | | |
| | 100 | 100 | 2 | | | | | |
| | 125 | 126 | 3 | | | | | |
| W104 | 30 | 94 | 6 | 70-130% | 9% | ≤15% | 15% | ≤15% |
| (mAb-2) | 40 | 96 | 4 | | | | | |
| | 50 | 107 | 5 | | | | | |
| | 75 | 98 | 6 | | | | | |
| | 100 | 100 | 2 | | | | | |
| | 125 | 118 | 6 | | | | | |

| | | | | | Specificity | |
|---|---|---|---|---|---|---|
| | Linearity | | | | | Ability to |
| Peptide | | ATP | | | Absence of | Detect |
| (Molecule) | R² | (R²) | Range | QL | Interference | Change |
| M105 | 0.9985 | ≥0.98 | 30-125% | 0.30% | No peaks ≥ | Ability to |
| (mAb-1) | | | | | QL in | detect |
| | | | | | interest | changes in |
| | | | | | region of | oxidation |
| | | | | | | level in |
| | | | | | | different |
| | | | | | | stressed |
| | | | | | | samples |
| W104 | 0.9883 | ≥0.98 | 30-125% | 0.30% | No peaks ≥ | Ability to |
| (mAb-2) | | | | | QL in | detect |
| | | | | | region of | changes in |
| | | | | | interest | oxidation |
| | | | | | | level in |
| | | | | | | different |
| | | | | | | stressed |
| | | | | | | samples |

TABLE 5
Applied variation of parameters of QDa based focused peptide mapping method

| | | Low | Set Point | High | Responses |
|---|---|---|---|---|---|
| Experiment type | Parameter | (−1) | (0) | (+1) | of Interest |
| Stage 1 DoE-1 | 1M DTT added in Denature/ | 18 | 20 | 22 | W104 and |
| (Sample Preparation) | Reduction Buffer (μL) | | | | M105 % |
| | Reduction Time (min) | 28 | 30 | 32 | Oxidation* |
| | Alkylation Time (min) | 28 | 30 | 32 | |
| | Lys-C Volume (μL) | 90 | 100 | 110 | |
| | Digestion Time (min) | 60 | 65 | 70 | |
| Stage 1 DoE-2 | % MPB (at starting gradient) | 15 | 16 | 17 | |
| (LC Instrument | % FA in Mobile Phases | 0.095 | 0.100 | 0.105 | |
| Method) | Column Temp (° C.) | 77 | 80 | 83 | |
| | Flow Rate (mL/min) | 0.29 | 0.30 | 0.31 | |
| Stage 2 | ESI Probe Temperature (° C.) | 390 | 400 | 410 | |
| | Capillary Voltage (kv) | 1.4 | 1.5 | NA* | |
| | Lys-C Lots | Lot 1 | Lot 2 | Lot 3 | |

TABLE 6
Results of mAb-1 M105 and mAb-2 W104 Oxidation % in co-
formulated drug product (Stage 1 DOE-I robustness study)

| | Reduction | Alkylation | Lys-C | Digestion | | |
|---|---|---|---|---|---|---|
| DTT (μL) | Time (min) | Time (min) | (μL) | Time (min) | W104 0x % | M105 ox % |
| 20 | 32 | 32 | 110 | 70 | 5.199 | 5.399 |
| 22 | 32 | 30 | 100 | 70 | 5.327 | 5.550 |
| 22 | 30 | 32 | 110 | 65 | 5.232 | 5.434 |
| 18 | 32 | 28 | 110 | 60 | 5.130 | 5.278 |
| 22 | 28 | 32 | 100 | 60 | 5.119 | 5.407 |
| 22 | 28 | 28 | 90 | 65 | 5.095 | 5.352 |
| 22 | 30 | 28 | 100 | 60 | 5.283 | 5.347 |
| 18 | 30 | 28 | 110 | 70 | 5.917 | 5.779 |
| 20 | 30 | 30 | 100 | 65 | 5.335 | 5.478 |
| 20 | 28 | 30 | 90 | 70 | 5.334 | 5.414 |
| 20 | 30 | 30 | 100 | 65 | 5.168 | 5.291 |
| 20 | 28 | 32 | 90 | 60 | 5.113 | 5.326 |
| 22 | 32 | 32 | 110 | 60 | 5.363 | 5.362 |
| 18 | 28 | 30 | 110 | 65 | 5.352 | 5.404 |
| 18 | 32 | 30 | 110 | 70 | 5.422 | 5.420 |
| 18 | 30 | 32 | 110 | 60 | 5.394 | 5.473 |
| 22 | 32 | 30 | 90 | 60 | 5.408 | 5.529 |
| 20 | 32 | 28 | 90 | 70 | 5.290 | 5.348 |
| 20 | 30 | 30 | 100 | 65 | 5.528 | 5.412 |
| 18 | 28 | 28 | 90 | 60 | 5.674 | 5.514 |
| 20 | 32 | 32 | 90 | 65 | 5.312 | 5.363 |
| 22 | 28 | 28 | 110 | 70 | 5.479 | 5.414 |
| 18 | 28 | 28 | 100 | 70 | 5.620 | 5.434 |
| 20 | 30 | 30 | 100 | 65 | 5.400 | 5.338 |
| 18 | 30 | 30 | 90 | 60 | 5.480 | 5.371 |
| 18 | 30 | 32 | 90 | 70 | 5.331 | 5.444 |
| 20 | 28 | 28 | 110 | 60 | 5.829 | 5.664 |
| 22 | 32 | 28 | 110 | 65 | 5.639 | 5.363 |
| 18 | 28 | 32 | 100 | 65 | 5.717 | 5.495 |
| 20 | 30 | 30 | 100 | 65 | 5.689 | 5.429 |
| 18 | 32 | 32 | 100 | 60 | 5.574 | 5.392 |
| 20 | 30 | 30 | 100 | 65 | 5.707 | 5.405 |
| 18 | 32 | 28 | 90 | 65 | 5.709 | 5.439 |
| 22 | 30 | 32 | 90 | 70 | 5.472 | 5.337 |
| 22 | 28 | 30 | 110 | 60 | 5.671 | 5.416 |
| 20 | 28 | 32 | 110 | 70 | 5.626 | 5.519 |

TABLE 7
Results of mAb-1 M105 and mAb-2 W104 Oxidation % in co-
formulated drug product (Stage 1 DOE-2 robustness study)

| % MPB | | | Column | Flow | | |
|---|---|---|---|---|---|---|
| (at starting | % FA | % FA | Temp | Rate | W104 | M105 |
| gradient) | (in water) | (in acetonitrile) | (° C.) | (mL/min) | 0x % | ox % |
| 15 | 0.095 | 0.105 | 83 | 0.31 | 5.045 | 5.072 |
| 16 | 0.100 | 0.100 | 80 | 0.31 | 4.868 | 5.175 |
| 17 | 0.105 | 0.095 | 77 | 0.31 | 4.886 | 5.113 |
| 17 | 0.095 | 0.095 | 83 | 0.31 | 5.055 | 5.096 |
| 17 | 0.105 | 0.105 | 77 | 0.29 | 4.791 | 5.148 |
| 16 | 0.100 | 0.100 | 80 | 0.30 | 4.764 | 5.084 |
| 16 | 0.100 | 0.105 | 80 | 0.30 | 4.850 | 5.026 |
| 17 | 0.095 | 0.105 | 83 | 0.29 | 4.972 | 4.885 |
| 15 | 0.100 | 0.100 | 80 | 0.30 | 4.977 | 5.133 |
| 16 | 0.100 | 0.100 | 77 | 0.30 | 4.933 | 5.132 |
| 15 | 0.105 | 0.105 | 83 | 0.29 | 5.145 | 5.299 |
| 15 | 0.095 | 0.095 | 77 | 0.31 | 4.952 | 5.255 |
| 15 | 0.105 | 0.105 | 77 | 0.31 | 4.745 | 5.147 |
| 16 | 0.100 | 0.100 | 80 | 0.30 | 4.881 | 5.033 |
| 16 | 0.100 | 0.095 | 80 | 0.30 | 5.124 | 5.053 |
| 16 | 0.100 | 0.100 | 80 | 0.30 | 5.013 | 5.092 |
| 17 | 0.095 | 0.095 | 77 | 0.29 | 4.858 | 5.149 |
| 15 | 0.095 | 0.095 | 77 | 0.31 | 4.797 | 5.251 |
| 16 | 0.095 | 0.100 | 80 | 0.30 | 5.024 | 5.166 |
| 15 | 0.095 | 0.105 | 77 | 0.29 | 5.045 | 5.349 |
| 17 | 0.095 | 0.105 | 77 | 0.31 | 5.027 | 5.036 |
| 17 | 0.105 | 0.095 | 83 | 0.29 | 5.075 | 4.910 |
| 16 | 0.100 | 0.100 | 80 | 0.30 | 5.121 | 5.101 |
| 15 | 0.095 | 0.095 | 83 | 0.29 | 5.373 | 5.023 |
| 16 | 0.100 | 0.100 | 80 | 0.29 | 5.242 | 5.099 |
| 16 | 0.100 | 0.100 | 80 | 0.30 | 5.192 | 5.039 |
| 16 | 0.105 | 0.100 | 80 | 0.30 | 5.295 | 5.199 |
| 17 | 0.095 | 0.095 | 77 | 0.29 | 5.179 | 5.136 |
| 15 | 0.105 | 0.095 | 77 | 0.29 | 5.172 | 5.392 |
| 17 | 0.105 | 0.105 | 83 | 0.31 | 5.265 | 4.817 |
| 15 | 0.095 | 0.095 | 83 | 0.29 | 5.406 | 5.087 |
| 16 | 0.100 | 0.100 | 80 | 0.30 | 5.522 | 5.208 |
| 16 | 0.100 | 0.100 | 83 | 0.30 | 5.624 | 5.019 |
| 17 | 0.100 | 0.100 | 80 | 0.30 | 5.333 | 4.962 |
| 15 | 0.105 | 0.095 | 83 | 0.31 | 5.661 | 5.251 |

TABLE 8
Results of Stage 2 Robustness Study (Probe
Temperature) for mAb-1 M105 and mAb-2 W104
Oxidation % in co-formulated drug product

| Probe Temperature(° C.) | Replicate | W104 ox % | M105 ox % |
|---|---|---|---|
| 400 | 1 | 4.923 | 5.104 |
| 400 | 2 | 5.191 | 5.202 |
| 400 | 3 | 4.963 | 5.119 |
| 390 | 1 | 4.963 | 5.154 |
| 390 | 2 | 5.006 | 5.142 |
| 390 | 3 | 4.930 | 5.135 |
| 410 | 1 | 5.117 | 5.152 |
| 410 | 2 | 4.931 | 5.167 |
| 410 | 3 | 4.932 | 5.175 |

TABLE 9
Results of Stage 2 Robustness Study (Capillary
Voltage) for mAb-1 M105 and mAb-2 W104 Oxidation
% in co-formulated drug product

| Capillary Voltage (kV) | Replicate | W104 ox % | M105 ox % |
|---|---|---|---|
| 1.5 | 1 | 5.439 | 5.185 |
| 1.5 | 2 | 5.351 | 5.200 |
| 1.5 | 3 | 5.231 | 5.177 |
| 1.4 | 1 | 5.456 | 5.222 |
| 1.4 | 2 | 5.276 | 5.170 |
| 1.4 | 3 | 5.274 | 5.166 |

TABLE 10
Results of Stage 2 Robustness Study (Lys-C Lot) for mAb-1 M105
and mAb-2 W104 Oxidation % in co-formulated drug product

| Lys-C Lot | Replicate | W104 ox % | M105 ox % |
|---|---|---|---|
| 1 | 1 | 4.736 | 5.142 |
| 1 | 2 | 4.696 | 5.147 |
| 1 | 3 | 4.720 | 5.091 |
| 2 | 1 | 4.812 | 5.163 |
| 2 | 2 | 4.817 | 5.167 |
| 2 | 3 | 4.767 | 5.215 |
| 3 | 1 | 4.734 | 5.139 |
| 3 | 2 | 4.769 | 5.152 |
| 3 | 3 | 4.830 | 5.150 |

### Example 2

The present Example describes exemplary methods for assessing critical quality attributes of a co-formulation comprising two different antibodies, i.e., mAb-3 (an antibody having a light chain comprising an amino acid sequence as set forth in SEQ ID NO:5 and a heavy chain comprising an amino acid sequence as set forth in SEQ ID NO:10) and mAb-4 (an antibody having a light chain comprising an amino acid sequence as set forth in SEQ ID NO:15 and a heavy chain comprising an amino acid sequence as set forth in SEQ ID NO:20).

### Chemicals and Recombinant Proteins

mAb-3 and mAb-4 were produced in Chinese hamster ovary (CHO) cells at Merck & Co., Inc. (Kenilworth, NJ, USA). The co-formulated drug product was made by mixing mAb-3 and mAb-4 at protein concentration ratios of 1:4. Sequencing grade modified trypsin was purchased from Promega Corporation. (Madison, WI). Dithiothreitol (DTT, no-weight format), and iodoacetamide (single use) supplied in amber tubes were obtained from Pierce Protein Biology (Thermo Scientific, Rockford, IL). Tris(hydroxymethyl)aminomethane hydrochloride (Tris-HCl) buffer (1 M), urea, sequencing grade trifluoroacetic acid (TFA), purified water (LC-MS grade), acetonitrile (LC-MS Grade), 0.1% formic acid in water (LC-MS Grade), and 0.1% formic acid in acetonitrile (LC-MS Grade) were obtained from Fisher Scientific (Waltham, MA).

Trypsin Digestion of mAbs

Samples were diluted with purified water to a concentration of 5 mg/mL for each antibody in a 1.5 mL polypropylene tube. The diluted sample (containing 100 μg of total protein in 4 μl) was denatured and reduced by mixing with 46 μL of Denature/Reduction Buffer (composed of 989 μL 8 M urea in 50 mM Tris-HCl and 11 μL 0.5 M DTT) in Thermomixer (Eppendorf, Hamburg, Germany) at 80° C. with 300 rpm shaking for 10 min. 342 μL Digestion Buffer (composed of 4784 μL 50 mM Tris-HCl and 56 μL 0.5 M DTT) and 8 μL of 0.5 μg/gL of sequencing grade modified trypsin were added, and the mix was allowed to incubate at 37° C. with 300 rpm shaking for 30 min. The digestion was quenched with 20% TFA before analysis.

### QDa Based LC-MS Analysis

Analysis was performed with a Waters ACQUITY Ultra Performance Liquid Chromatography (UPLC) H-Class System equipped with an ACQUITY QDa detector (Waters, Milford, MA). QDa mass spectrometer was operated in the positive Selective Ion Recording (SIR) mode set up with specific m/z channels aimed to detect targeted native and oxidized peptides (Table 11). The QDa was operated with the settings of cone voltage of 15 V, probe temperature of 600° C., capillary voltage of 1.5 kV of sampling Rate of 5 points/sec.

A HALO® Peptide ES-C18 Column (2.7 μm, 160 Å, 2.1 mm×150 mm; HALO, Wilmington, DE) was used for chromatography separation. Aqueous formic acid (0.1%) was used as mobile phase A (MPA), and 0.1% formic acid in acetonitrile as mobile phase B (MPB). Injection volume was 10 μL. Peaks were eluted using the following gradients. 12% to 25% MPB in 12 minutes (at a flow rate of 0.6 mL/min), 25% to 95% MPB in 0.2 minute (at a flow rate of 0.8 mL/min). The column was then washed with 95% MPB for 1.8 minutes (at a flow rate of 0.8 mL/min) and re-equilibrated with 12% MPB for 3.9 minutes (2.9 minutes at a flow rate of 0.8 mL/min and 1.0 minutes at a flow rate of 0.6 mL/min). The column temperature was set at 65° C. A diverter valve was used to switch the flow from the column to QDa within 5.00-12.00 min only of the gradients to minimize detector contamination. Peak ID of each SIR channel was also confirmed by running the same chromatography conditions with HRMS (Thermo Scientific™ Q Exactive™ HF-X, Thermo Scientific, Waltham, MA).

### Data Processing, Calculation, and Statistical Analysis

Data were acquired and processed by using custom field automated calculation with Empower software (Waters, Milford, MA). MassLynx V4.1 (Waters, Milford, MA) was used to calculate the theoretical m/z for the peptide of interest. The component peak areas of all SIR channels, including mAb-3 M105 oxidized peptides (+16 Da for methionine sulfoxide, including M105_103+16 oxidation (SIR Ch9), and missed cleavage peptide M105_99 +16 oxidation (SIR Ch7)), mAb-4 W102 oxidized peptides (+4, +16, and +32 Da for kynurenine, hydroxytryptophan, and N-formylkynurenine, respectively), and the corresponding native non-oxidized M105 and W102 peptides, were determined. Component peak areas from the QDa SIR channels were used to calculate the percent oxidation (% oxidation) by dividing the sum of oxidized component peak areas by the sum of oxidized and non-oxidized (native peptide) component peak areas.

Design of Experiment (DoE) for assessing the impact of sample preparation parameters was generated by using JMP 15.2.0 (JMP Statistical Discovery LLC., Cary, NC). DoE for evaluating the impact of instrument method parameters was generated by using Fusion Method Development™ Software (S-Matrix, Eureka, CA). All statistical analyses were performed using JMP 15.2.0.

### Development of a QDa Based Peptide Mapping Method

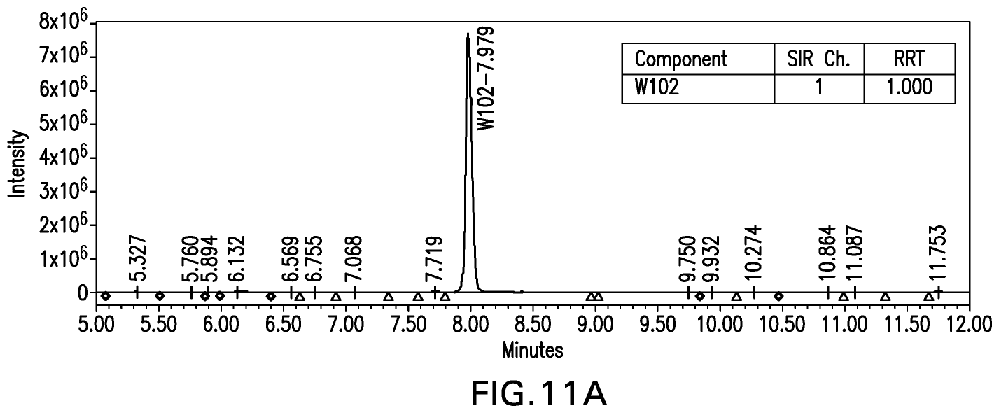

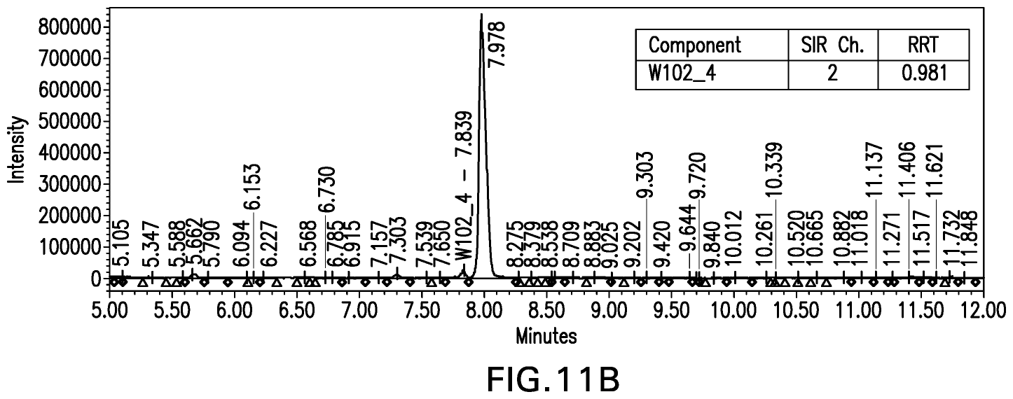

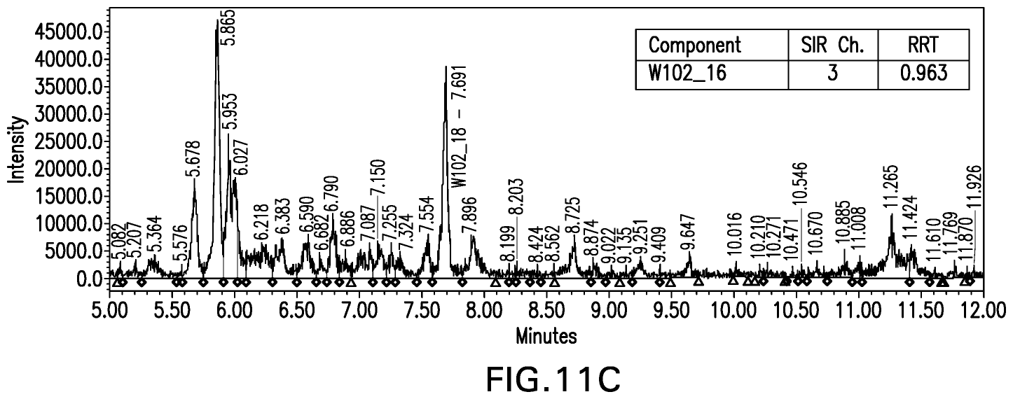

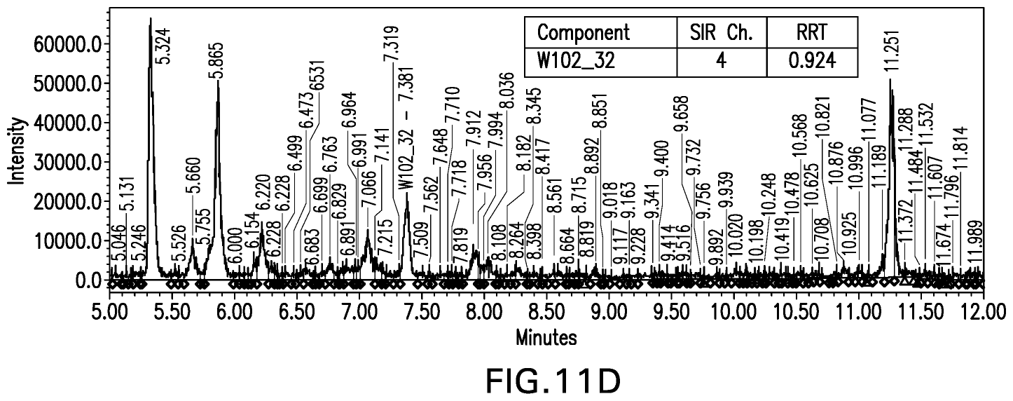

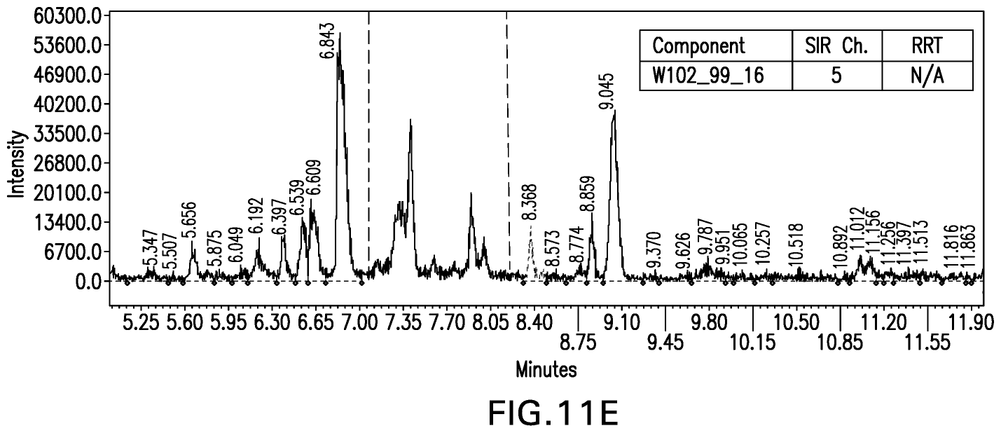

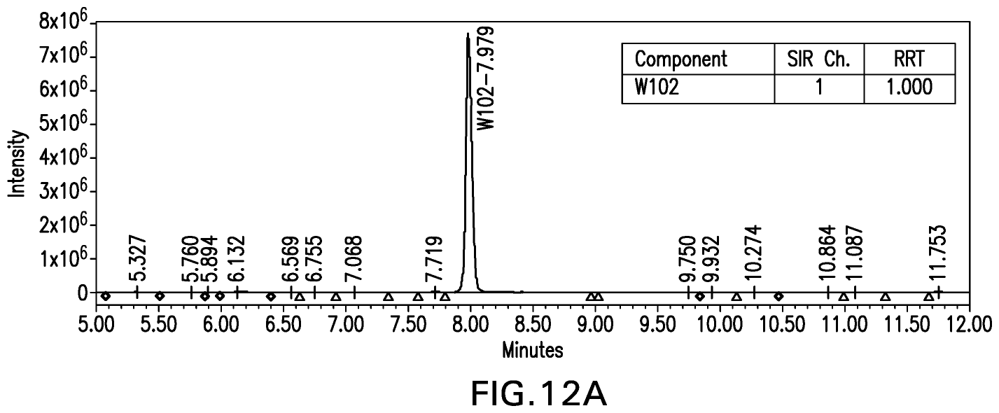

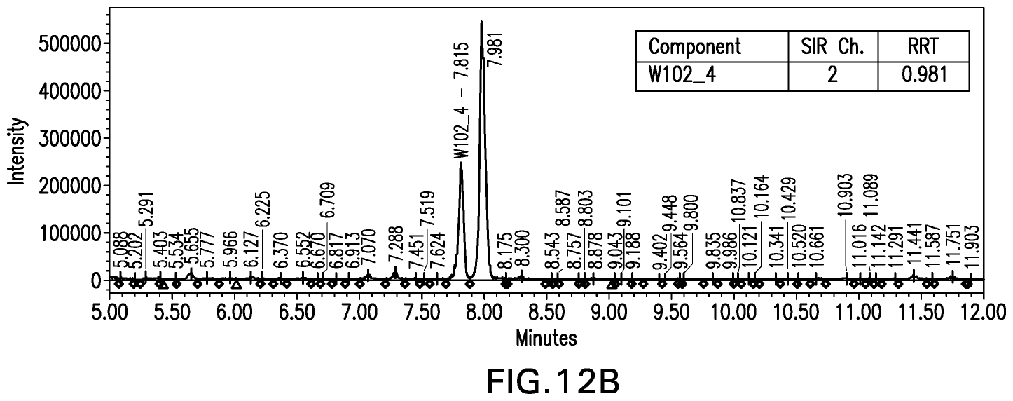

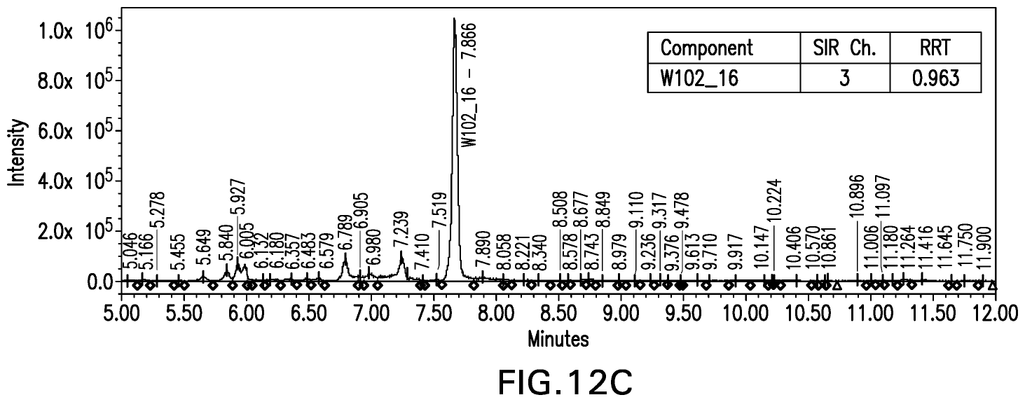

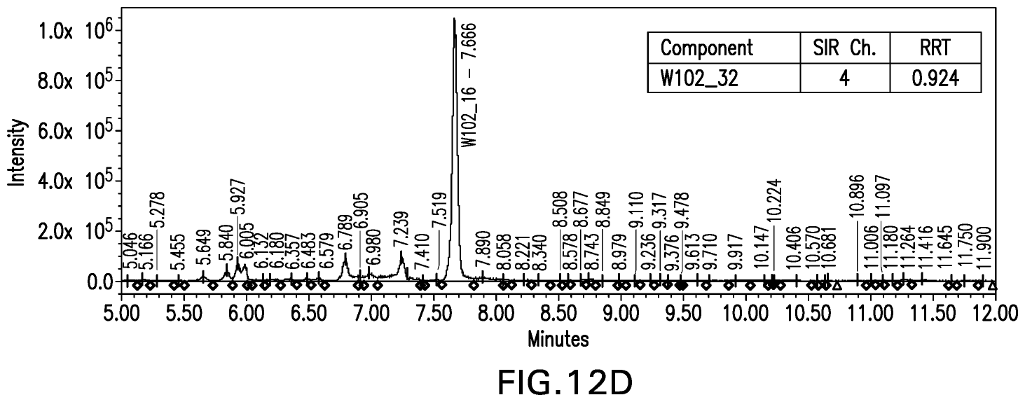

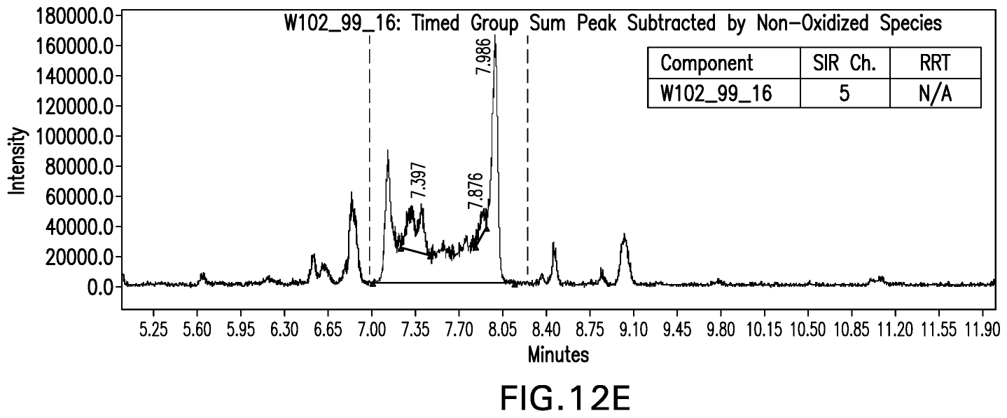

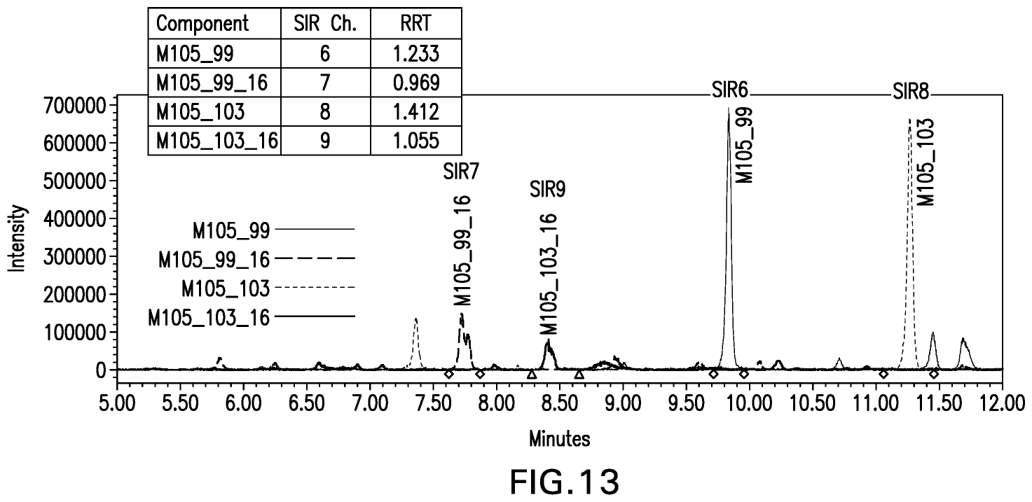

Nine Channels were Set to Detect Both Non-Oxidized and Oxidized Forms of the relevant peptides, using the +3 charge state of each species for mAb-4, and +3 charge state of peptide M105_99 and +2 charge state of peptide M105_103 for mAb-3. Representative SIR channels are shown in FIGS. 11-13. The non-oxidized M105 peptides from mAb-3 were detected by SIR at m/z 992.83 (peptide 99-124) and 1193.54 (peptide 103-124). The oxidized M105 peptides (+16 Da) from mAb-3 were detected by SIR at m/z 998.27 (peptide 99-124) and 1201.54 (peptide 103-124). The non-oxidized W102 peptide from mAb-4 were detected by SIR at m/z 785.70 (peptide 102-123). The oxidized W102 peptides (+4, +16, +32 Da) from mAb-4 were detected by SIR at m/z 787.04 (peptide 102-123, +4 Da), m/z 791.10 (peptide 102-123, +16 Da), m/z 796.35 (peptide 102-123, +32 Da), m/z 935.45 (peptide 99-123, +16 Da). The peptide peaks unrelated to oxidation were excluded from calculations to determine % oxidation.

TABLE 11
Channel name, peptide nomenclature,
and m/z monitored for each mAb

| mAb | Channel Name | Peptide | m/z monitored |
|---|---|---|---|
| mAb-4 | SIR Ch1 | W102 | 785.70 |
| mAb-4 | SIR Ch2 | W102ox_4 | 787.04 |
| mAb-4 | SIR Ch3 | W102ox_16 | 791.10 |
| mAb-4 | SIR Ch4 | W102ox_32 | 796.35 |
| mAb-4 | SIR Ch5 | W102ox_99_16 | 935.45 |
| mAb-3 | SIR Ch6 | M105_99 | 992.83 |
| mAb-3 | SIR Ch7 | M105ox_99_16 | 998.27 |
| mAb-3 | SIR Ch8 | M105_103 | 1193.54 |
| mAb-3 | SIR Ch9 | M10Sox_103_16 | 1201.54 |

### Example 3

The present Example describes exemplary methods for assessing critical quality attributes of a co-formulation comprising two different antibodies, i.e., mAb-5 (an antibody having a light chain comprising an amino acid sequence as set forth in SEQ ID NO:5 and a heavy chain comprising an amino acid sequence as set forth in SEQ ID NO: 10) and mAb-6 (an antibody having a light chain comprising an amino acid sequence as set forth in SEQ ID NO:109 and a heavy chain comprising an amino acid sequence as set forth in SEQ ID NO: 114).

### Chemicals and Recombinant Proteins

mAb-5 and mAb-6 were produced in Chinese hamster ovary (CHO) cells at Merck & Co., Inc. (Kenilworth, NJ, USA). A co-formulated drug product was made by mixing mAb-5 and mAb-6 at protein concentration ratios of 1:4. Lysyl Endopeptidase (Lys-C) was purchased from FUJIFILM Wako Chemicals U.S.A. (Richmond, VA). Dithiothreitol (DTT, no-weight format), and iodoacetamide (single use) supplied in amber tubes were obtained from Pierce Protein Biology (Thermo Scientific, Rockford, IL). Tris(hydroxymethyl)aminomethane hydrochloride (Tris-HCl) buffer (1 M), guanidine hydrochloride (GuHCl), sequencing grade trifluoroacetic acid (TFA), purified water (LC-MS grade), acetonitrile (LC-MS Grade), 0.1% formic acid in water (LC-MS Grade), and 0.1% formic acid in acetonitrile (LC-MS Grade) were obtained from Fisher Scientific (Waltham, MA). Ethylenediaminetetraacetic Acid (EDTA) (0.5 M) was purchased from Promega (Madison, WI).

Lys-C Digestion of mAbs

Samples were diluted with purified water to a concentration of 5 mg/mL for each antibody in a 1.5 mL polypropylene tube. The diluted sample (containing 100 μg of total protein in 20 μl) was denatured and reduced by mixing with 83 μL of Denature/Reduction Buffer (composed of 750 μL 8 M GuHCl, 50 μL 1 M Tris-HCl, 10 μL 0.5 M EDTA, and 20 μL 1 M DTT) in Thermomixer (Eppendorf, Hamburg, Germany) at 37° C. with 300 rpm shaking for 30 min. Iodoacetamide (5 μL 1 M) was added to initiate alkylation for 30 min under dark. Following the alkylation, 400 μL Digestion Buffer (composed of 500 μL 1 M Tris-HCl, 100 μL 0.5 M EDTA, 20 μL 1 M DTT, and 9.38 mL purified water) and 100 μL of diluted Lys-C enzyme Solution (composed of one vial of Lyophilized Lys-C and 500 μL of Digestion Buffer) were added, and the mix was allowed to incubate at 37° C. with 300 rpm shaking for 65 min. The digestion was quenched with 20% TFA before analysis.

### QDa Based LC-MS Analysis

Analysis was performed with a Waters ACQUITY Ultra Performance Liquid Chromatography (UPLC) H-Class System equipped with an ACQUITY QDa detector (Waters, Milford, MA). QDa mass spectrometer was operated in the positive Selective Ion Recording (SIR) mode set up with specific m/z channels aimed to detect targeted native and oxidized peptides (Table 12). The QDa was operated with the settings of cone voltage of 15 V, probe temperature of 400° C., capillary voltage of 1.5 kV of sampling Rate of 2 points/sec.

A Waters ACQUITY Premier Peptide BEH C18 Column (1.7 μm, 130A, 2.1 mm×150 mm; Waters, Milford, MA) was used for chromatography separation. Aqueous formic acid (0.1%) was used as mobile phase A (MPA), and 0.1% formic acid in acetonitrile as mobile phase B (MPB). Injection volume was 10 μL. Peaks were eluted using the following gradients: 15% MPB for 2 minutes, 15% to 37% MPB in 18 minutes, 37% to 95% MPB in 1 minute. The column was then washed with 95% MPB for 4 minutes and re-equilibrated with 15% MPB for 8 minutes. Flow rate was 0.45 mL/min. The column temperature was set at 60° C.

### Data Processing, Calculation, and Statistical Analysis

Data were acquired and processed by using custom field automated calculation with Empower software (Waters, Milford, MA). MassLynx V4.1 (Waters, Milford, MA) was used to calculate the theoretical m/z for the peptide of interest. The component peak areas of all SIR channels, including mAb-5 M105 oxidized peptides (+16 Da for methionine sulfoxide). mAb-6 W102 oxidized peptides (+4, +16, and +32 Da for kynurenine, hydroxytryptophan, and N-formylkynurenine, respectively), mAb-6 W7 oxidized peptides (+4, +16, and +32 Da for kynurenine, hydroxytryptophan, and N-formylkynurenine, respectively), and the corresponding native non-oxidized M105, W102, and W7 peptides, were determined (Table 12). Component peak areas from the QDa SIR channels were used to calculate the percent oxidation (% oxidation) by dividing the sum of oxidized component peak areas by the sum of oxidized and non-oxidized (native peptide) component peak areas.

### Development of a QDa Based Peptide Mapping Method

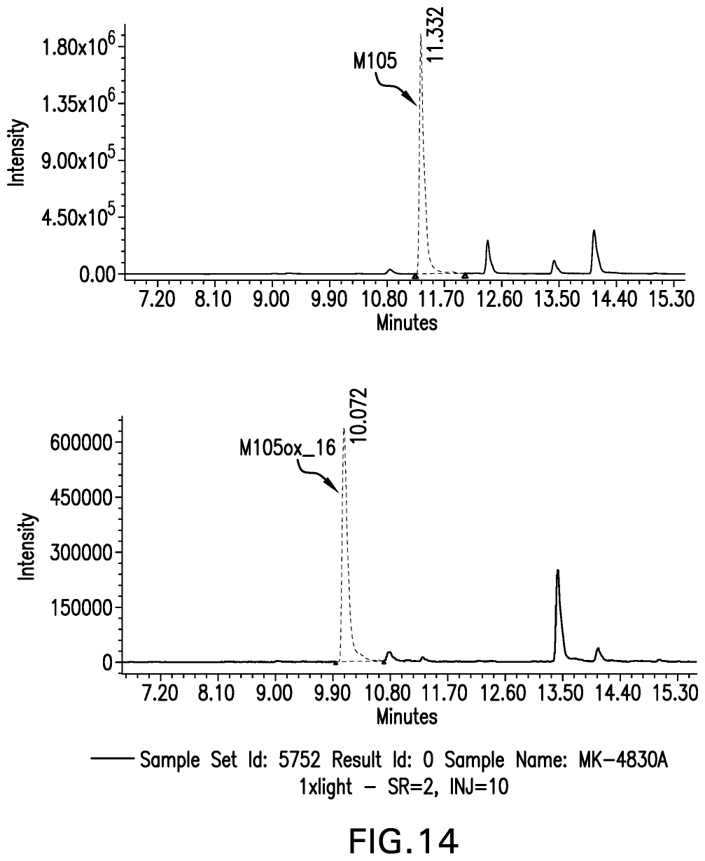

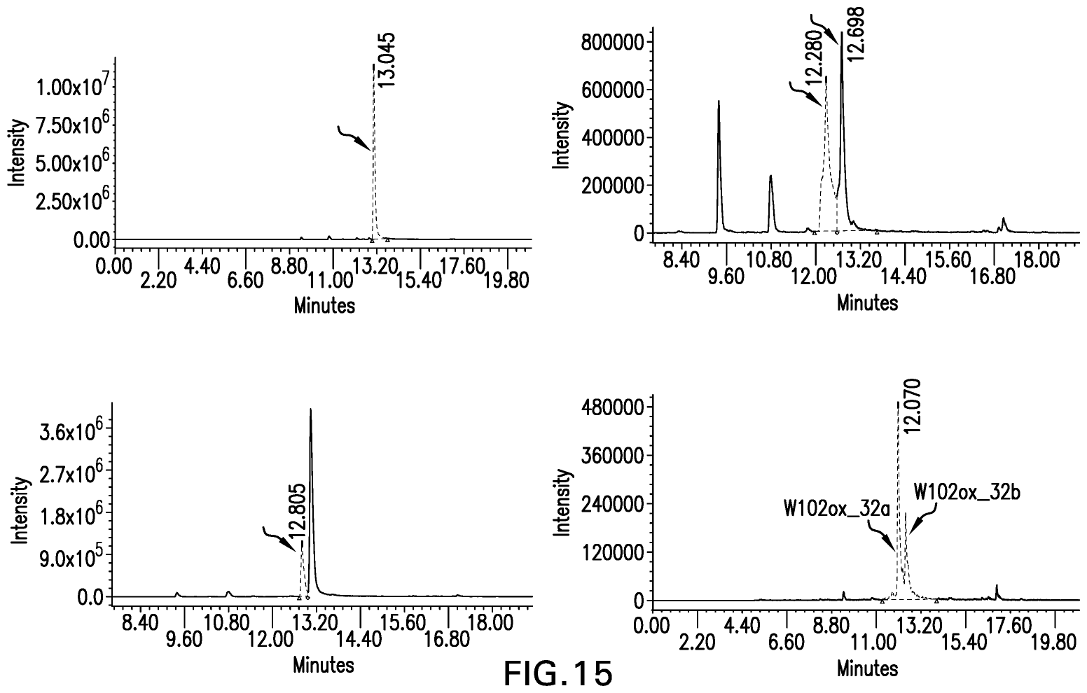

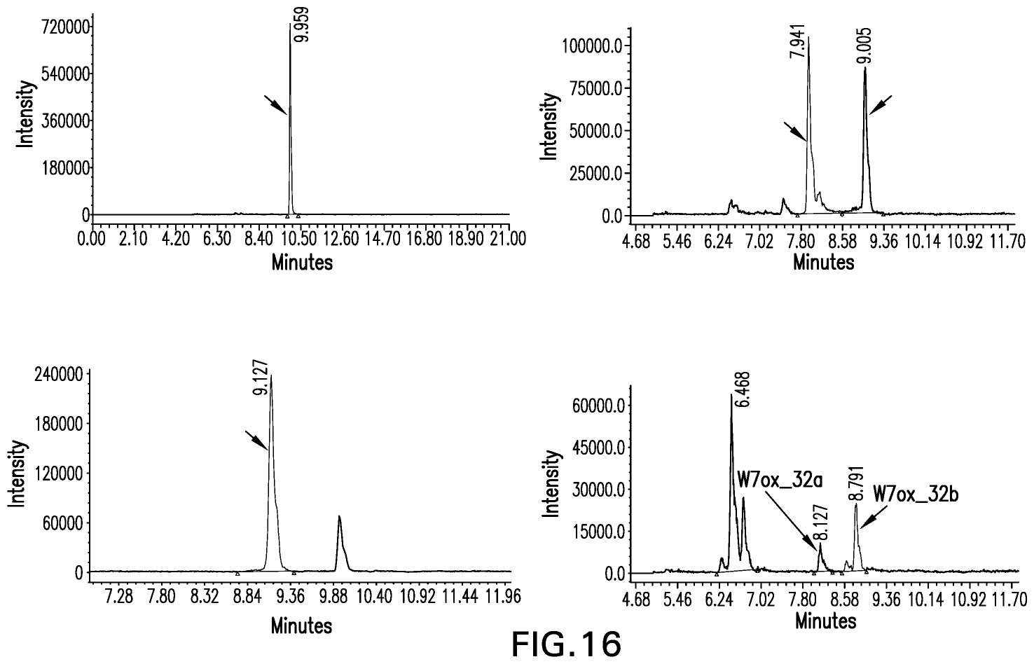

Ten channels were set to detect both non-oxidized and oxidized forms of the relevant peptides, using the +6 charge state for W102 of mAb-6, +3 charge state for W7 of mAb-6, and +5 charge state for M105 of mAb-5. Representative SIR channels are shown in FIGS. 14-16. The non-oxidized M105 peptides from mAb-5 were detected by SIR at m/z 934.27, and the oxidized M10S peptides (+16 Da) from mAb-5 were detected by SIR at m/z 937.50. The non-oxidized W102 peptide from mAb-6 were detected by SIR at m/z 822.52. The oxidized W102 peptides (+4, +16, +32 Da) from mAb-6 were detected by SIR at m/z 823.18, m/z 825.18, and m/z 827.85. The non-oxidized W7 peptide from mAb-6 were detected by SIR at m/z 490.63. The oxidized W7 peptides (+4, +16, +32 Da) from mAb-6 were detected by SIR at m/z 491.95, m/z 495.95, and m/z 501.29. The peptide peaks unrelated to oxidation were excluded from calculations to determine % oxidation.

TABLE 12
Channel name, peptide nomenclature,
and m/z monitored for each mAb

| mAb | Channel Name | Peptide | m/z monitored |
|---|---|---|---|
| mAb-5 | SIR Ch1 | M105 | 934.27 |
| mAb-5 | SIR Ch2 | M105ox_16 | 937.50 |
| mAb-6 | SIR Ch3 | W102 | 822.52 |
| mAb-6 | SIR Ch4 | W102ox_4 | 823.18 |
| mAb-6 | SIR Ch5 | W102ox_16 | 825.18 |
| mAb-6 | SIR Ch6 | W102ox_32 | 827.85 |
| mAb-6 | SIR Ch7 | W7 | 490.63 |
| mAb-6 | SIR Ch8 | W7ox_4 | 491.95 |
| mAb-6 | SIR Ch9 | W7ox_16 | 495.95 |
| mAb-6 | SIR Ch10 | W7ox_32 | 501.29 |

Table 13 below summarizes all sequences disclosed in the present specification.

TABLE 13
SEQ ID NOS and Corresponding Sequences

| SEQ | | |
|---|---|---|
| ID NO | Description | Sequence |
| 1 | Pembrolizumab, | RASKGVSTSGYSYLH |
| | VL-CDR1 | |
| 2 | Pembrolizumab, | LASYLES |
| | VL-CDR2 | |
| 3 | Pembrolizumab, | QHSRDLPLT |
| | VL-CDR3 | |
| 4 | Pembrolizumab, | EIVLTQSPATLSLSPGERATLSCRASKGVSTSGYSYLHWYQQKPGQAPRLL |
| | VL | IYLASYLESGVPARFSGSGSGTDFTLTISSLEPEDFAVYYCQHSRDLPLTFG |
| | | GGTKVEIK |
| 5 | Pembrolizumab, | EIVLTQSPATLSLSPGERATLSCRASKGVSTSGYSYLHWYQQKPGQAPRLL |
| | light chain | IYLASYLESGVPARFSGSGSGTDFTLTISSLEPEDFAVYYCQHSRDLPLTFG |
| | | GGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTH |
| | | QGLSSPVTKSFNRGEC |
| 6 | Pembrolizumab, | NYYMY |
| | VH-CDR1 | |
| 7 | Pembrolizumab, | GINPSNGGTNFNEKFKN |
| | VH-CDR2 | |
| 8 | Pembrolizumab, | RDYRFDMGEDY |
| | VH-CDR3 | |
| 9 | Pembrolizumab, | QVQLVQSGVEVKKPGASVKVSCKASGYTFTNYYMYWVRQAPGQGLEW |
| | VH | MGGINPSNGGTNFNEKFKNRVTLTTDSSTTTAYMELKSLQFDDTAVYYC |
| | | ARRDYRFDMGFDYWGQGTTVTVSS |
| 10 | Pembrolizumab, | QVQLVQSGVEVKKPGASVKVSCKASGYTFTNYYMYWVRQAPGQGLEW |
| | heavy chain | MGGINPSNGGTNFNEKFKNRVTLTTDSSTTTAYMELKSLQFDDTAVYYC |
| | | ARRDYRFDMGFDYWGQGTTVTVSSASTKGPSVFPLAPCSRSTSESTAALG |
| | | CLVKDYFPEPVTVSWNSGALTSGVHTFPAVLQSSGLYSLSSVVTVPSSSLG |
| | | TKTYTCNVDHKPSNTKVDKRVESKYGPPCPPCPAPEFLGGPSVFLFPPKPK |
| | | DTLMISRTPEVTCVVVDVSQEDPEVQFNWYVDGVEVHNAKTKPREEQFN |
| | | STYRVVSVLTVLHQDWLNGKEYKCKVSNKGLPSSIEKTISKAKGQPREPQ |
| | | VYTLPPSQEEMTKNQVSLTCLVKGFYPSDIAVEWESNGQPENNYKTTPPV |
| | | LDSDGSFFLYSRLTVDKSRWQEGNVFSCSVMHEALHNHYTQKSLSLSLG |
| | | K |
| 11 | Anti-LAG3 Ab6, | KASQSLDYEGDSDMN |
| | VL-CDR1 | |
| 12 | Anti-LAG3 Ab6, | GASNLES |
| | VL-CDR2 | |
| 13 | Anti-LAG3 Ab6, | QQSTEDPRT |
| | VL-CDR3 | |
| 14 | Anti-LAG3 Ab6, | DIVMTQTPLSLSVTPGQPASISCKASQSLDYEGDSDMNWYLQKPGQPPQL |
| | VL | LIYGASNLESGVPDRFSGSGSGTDFTLKISRVEAEDVGVYYCQQSTEDPRT |
| | | FGGGTKVEIK |
| 15 | Anti-LAG3 Ab6, | DIVMTQTPLSLSVTPGQPASISCKASQSLDYEGDSDMNWYLQKPGQPPQL |
| | light chain | LIYGASNLESGVPDRFSGSGSGTDFTLKISRVEAEDVGVYYCQQSTEDPRT |
| | | FGGGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEV |
| | | THQGLSSPVTKSFNRGEC |
| 16 | Anti-LAG3 Ab6, | DYNVD |
| | VH-CDR1 | |
| 17 | Anti-LAG3 Ab6, | DINPNDGGTIYAQKFQE |
| | VH-CDR2 | |
| 18 | Anti-LAG3 Ab6, | NYRWFGAMDH |
| | VH-CDR3 | |
| 19 | Anti-LAG3 Ab6, | QMQLVQSGPEVKKPGTSVKVSCKASGYTFTDYNVDWVRQARGQRLEWI |
| | VH | GDINPNDGGTIYAQKFQERVTITVDKSTSTAYMELSSLRSEDTAVYYCAR |
| | | NYRWFGAMDHWGQGTTVTVSS |
| 20 | Anti-LAG3 Ab6, | QMQLVQSGPEVKKPGTSVKVSCKASGYTFTDYNVDWVRQARGQRLEWI |
| | heavy chain | GDINPNDGGTIYAQKFQERVTITVDKSTSTAYMELSSLRSEDTAVYYCAR |
| | | NYRWFGAMDHWGQGTTVTVSSASTKGPSVFPLAPCSRSTSESTAALGCL |
| | | VKDYFPEPVTVSWNSGALTSGVHTFPAVLQSSGLYSLSSVVTVPSSSLGT |
| | | KTYTCNVDHKPSNTKVDKRVESKYGPPCPPCPAPEFLGGPSVFLFPPKPK |
| | | DTLMISRTPEVTCVVVDVSQEDPEVQFNWYVDGVEVHNAKTKPREEQFN |
| | | STYRVVSVLTVLHQDWLNGKEYKCKVSNKGLPSSIEKTISKAKGQPREPQ |
| | | VYTLPPSQEEMTKNQVSLTCLVKGFYPSDIAVEWESNGQPENNYKTTPPV |
| | | LDSDGSFFLYSRLTVDKSRWQEGNVFSCSVMHEALHNHYTQKSLSLSLG |
| | | K |
| 21 | Anti-TIGIT | RASEHIYSYLS |
| | antibody, VL- | |
| | CDR1 | |
| 22 | Anti-TIGIT | NAKTLAE |
| | antibody, VL- | |
| | CDR2 | |
| 23 | Anti-TIGIT | QHHFGSPLT |
| | antibody, VL- | |
| | CDR3 | |
| 24 | Anti-TIGIT | DIQMTQSPSSLSASVGDRVTITCRASEHIYSYLSWYQQKPGKVPKLLIYNA |
| | antibody, VL | KTLAEGVPSRFSGSGSGTDFTLTISSLQPEDVATYYCQHHFGSPLTFGQGT |
| | | RLEIK |
| 25 | Anti-TIGIT | DIQMTQSPSSLSASVGDRVTITCRASEHTYSYLSWYQQKPGKVPKLLIYNA |
| | antibody, light | KTLAEGVPSRESGSGSGTDFTLTISSLQPEDVATYYCQHHFGSPLTFGQGT |
| | chain | RLEIKRTVAAPSVFIFPPSDEQLKSGTASVVCLLNNFYPREAKVQWKVDN |
| | | ALQSGNSQESVTEQDSKDSTYSLSSTLTLSKADYEKHKVYACEVTHQGLS |
| | | SPVTKSFNRGEC |
| 26 | Anti-TIGIT | SYVMH |
| | antibody, VH- | |
| | CDR1 | |
| 27 | Anti-TIGIT | YIDPYNDGAKYAQKFQG |
| | antibody, VH- | |
| | CDR2 | |
| 28 | Anti-TIGIT | GGPYGWYFDV |
| | antibody, VH- | |
| | CDR3 | |
| 29 | Anti-TIGIT | EVQLVQSGAEVKKPGSSVKVSCKASGYTFSSYVMHWVRQAPGQGLEWI |
| | antibody, VH | GYIDPYNDGAKYAQKFQGRVTLTSDKSTSTAYMELSSLRSEDTAVYYCA |
| | | RGGPYGWYFDVWGQGTTVTVSS |
| 30 | Anti-TIGIT | EVQLVQSGAEVKKPGSSVKVSCKASGYTFSSYVMHWVRQAPGQGLEWI |
| | antibody, heavy | GYIDPYNDGAKYAQKFQGRVTLTSDKSTSTAYMELSSLRSEDTAVYYCA |
| | chain | RGGPYGWYFDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLGT |
| | | QTYICNVNHKPSNTKVDKKVEPKSCDKTHTCPPCPAPELLGGPSVFLFPPK |
| | | PKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPR |
| | | EPQVYTLPPSRDELTKNQVSLTCLVKGFYPSDIAVEWESNGQPENNYKTT |
| | | PPVLDSDGSFFLYSKLTVDKSRWQQGNVFSCSVMHEALHNHYTQKSLSL |
| | | SPGK |
| 31 | Nivolumab, VL- | RASQSVSSYLA |
| | CDR1 | |
| 32 | Nivolumab, VL- | DASNRAT |
| | CDR2 | |
| 33 | Nivolumab, VL- | QQSSNWPRT |
| | CDR3 | |
| 34 | Nivolumab, VL | EIVLTQSPATLSLSPGERATLSCRASQSVSSYLAWYQQKPGQAPRLLIYDA |
| | | SNRATGIPARFSGSGSGTDFTLTISSLEPEDFAVYYCQQSSNWPRTFGQGT |
| | | KVEIK |
| 35 | Nivolumab, light | EIVLTQSPATLSLSPGERATLSCRASQSVSSYLAWYQQKPGQAPRLLIYDA |
| | chain | SNRATGIPARFSGSGSGTDFTLTISSLEPEDFAVYYCQQSSNWPRTFGQGT |
| | | KVEIKRTVAAPSVFIFPPSDEQLKSGTASVVCLLNNFYPREAKVQWKVDN |
| | | ALQSGNSQESVTEQDSKDSTYSLSSTLTLSKADYEKHKVYACEVTHQGLS |
| | | SPVTKSFNRGEC |
| 36 | Nivolumab, VH- | NSGMH |
| | CDR1 | |
| 37 | Nivolumab, VH- | VIWYDGSKRYYADSVKG |
| | CDR2 | |
| 38 | Nivolumab, VH- | NDDY |
| | CDR3 | |
| 39 | Nivolumab, VH | QVQLVESGGGVVQPGRSLRLDCKASGITFSNSGMHWVRQAPGKGLEWV |
| | | AVIWYDGSKRYYADSVKGRFTISRDNSKNTLFLQMNSLRAEDTAVYYCA |
| | | TNDDYWGQGTLVTVSS |
| 40 | Nivolumab, heavy | QVQLVESGGGVVQPGRSLRLDCKASGITFSNSGMHWVRQAPGKGLEWV |
| | chain | AVIWYDGSKRYYADSVKGRFTISRDNSKNTLFLQMNSLRAEDTAVYYCA |
| | | TNDDYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVVS |
| | | VLTVLHQDWLNGKEYKCKVSNKGLPSSIEKTISKAKGQPREPQVYTLPPS |
| | | QEEMTKNQVSLTCLVKGFYPSDIAVEWESNGQPENNYKTTPPVLDSDGSF |
| | | FLYSRLTVDKSRWQEGNVFSCSVMHEALHNHYTQKSLSLSLGK |
| 41 | Anti-LAG3 Ab9, | DINPNGGGTIYAQKFQE |
| | VH-CDR2 | |
| 42 | Anti-LAG3 Ab8, | DINPNQGGTIYAQKFQE |
| | VH-CDR2 | |
| 43 | Anti-LAG3 Ab7, | DINPNSGGTIYAQKFQE |
| | VH-CDR2 | |
| 44 | Anti-LAG3 Ab5, | DINPNNGGTIYAQKFQE |
| | VH-CDR2 | |
| 45 | Anti-LAG3 Ab9, | QMQLVQSGPEVKKPGTSVKVSCKASGYTFTDYNVDWVRQARGQRLEWI |
| | VH | GDINPNGGGTIYAQKFQERVTITVDKSTSTAYMELSSLRSEDTAVYYCAR |
| | | NYRWFGAMDHWGQGTTVTVSS |
| 46 | Anti-LAG3 Ab8, | QMQLVQSGPEVKKPGTSVKVSCKASGYTFTDYNVDWVRQARGQRLEWI |
| | VH | GDINPNQGGTIYAQKFQERVTITVDKSTSTAYMELSSLRSEDTAVYYCAR |
| | | NYRWFGAMDHWGQGTTVTVSS |
| 47 | Anti-LAG3 Ab7, | QMQLVQSGPEVKKPGTSVKVSCKASGYTFTDYNVDWVRQARGQRLEWI |
| | VH | GDINPNSGGTIYAQKFQERVTITVDKSTSTAYMELSSLRSEDTAVYYCAR |
| | | NYRWFGAMDHWGQGTTVTVSS |
| 48 | Anti-LAG3 Abs, | QMQLVQSGPEVKKPGTSVKVSCKASGYTFTDYNVDWVRQARGQRLEWI |
| | VH | GDINPNNGGTIYAQKFQERVTITVDKSTSTAYMELSSLRSEDTAVYYCAR |
| | | NYRWFGAMDHWGQGTTVTVSS |
| 49 | Anti-LAG3 | QMQLVQSGPEVKKPGTSVKVSCKASGYTFTDYNVDWVRQARGQRLEWI |
| | variant, IgG1 HC | GDINPNGGGTIYAQKFQERVTITVDKSTSTAYMELSSLRSEDTAVYYCAR |
| | with VH-CDR2 of | NYRWFGAMDHWGQGTTVTVSSASTKGPSVFPLAPSSKSTSGGTAALGCL |
| | Ab9 | VKDYFPEPVTVSWNSGALTSGVHTFPAVLQSSGLYSLSSVVTVPSSSLGT |
| | | QTYICNVNHKPSNTKVDKKVEPKSCDKTHTCPPCPAPELLGGPSVFLFPPK |
| | | PKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPR |
| | | EPQVYTLPPSRDELTKNQVSLTCLVKGFYPSDIAVEWESNGQPENNYKTT |
| | | PPVLDSDGSFFLYSKLTVDKSRWQQGNVFSCSVMHEALHNHYTQKSLSL |
| | | SPGK |
| 50 | Anti-LAG3 Ab4, | QMQLVQSGPEVKKPGTSVKVSCKASGYTFTDYNVDWVRQARGQRLEWI |
| | IgG1 HC | GDINPNQGGTIYAQKFQERVTITVDKSTSTAYMELSSLRSEDTAVYYCAR |
| | | NYRWFGAMDHWGQGTTVTVSSASTKGPSVFPLAPSSKSTSGGTAALGCL |
| | | VKDYFPEPVTVSWNSGALTSGVHTFPAVLQSSGLYSLSSVVTVPSSSLGT |
| | | QTYICNVNHKPSNTKVDKKVEPKSCDKTHTCPPCPAPELLGGPSVFLFPPK |
| | | PKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPR |
| | | EPQVYTLPPSRDELTKNQVSLTCLVKGFYPSDIAVEWESNGQPENNYKTT |
| | | PPVLDSDGSFFLYSKLTVDKSRWQQGNVFSCSVMHEALHNHYTQKSLSL |
| | | SPGK |
| 51 | Anti-LAG3 Ab2, | QMQLVQSGPEVKKPGTSVKVSCKASGYTFTDYNVDWVRQARGQRLEWI |
| | IgG1 HC | GDINPNSGGTIYAQKFQERVTITVDKSTSTAYMELSSLRSEDTAVYYCAR |
| | | NYRWFGAMDHWGQGTTVTVSSASTKGPSVFPLAPSSKSTSGGTAALGCL |
| | | VKDYFPEPVTVSWNSGALTSGVHTFPAVLQSSGLYSLSSVVTVPSSSLGT |
| | | QTYICNVNHKPSNTKVDKKVEPKSCDKTHTCPPCPAPELLGGPSVFLFPPK |
| | | PKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPR |
| | | EPQVYTLPPSRDELTKNQVSLTCLVKGFYPSDIAVEWESNGQPENNYKTT |
| | | PPVLDSDGSFFLYSKLTVDKSRWQQGNVFSCSVMHEALHNHYTQKSLSL |
| | | SPGK |
| 52 | Anti-LAG3 Ab3, | QMQLVQSGPEVKKPGTSVKVSCKASGYTFTDYNVDWVRQARGQRLEWI |
| | IgG1 HC | GDINPNDGGTIYAQKFQERVTITVDKSTSTAYMELSSLRSEDTAVYYCAR |
| | | NYRWFGAMDHWGQGTTVTVSSASTKGPSVFPLAPSSKSTSGGTAALGCL |
| | | VKDYFPEPVTVSWNSGALTSGVHTFPAVLQSSGLYSLSSVVTVPSSSLGT |
| | | QTYICNVNHKPSNTKVDKKVEPKSCDKTHTCPPCPAPELLGGPSVELFPPK |
| | | PKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPR |
| | | EPQVYTLPPSRDELTKNQVSLTCLVKGFYPSDIAVEWESNGQPENNYKTT |
| | | PPVLDSDGSFFLYSKLTVDKSRWQQGNVFSCSVMHEALHNHYTQKSLSL |
| | | SPGK |
| 53 | Anti-LAG3 Ab1, | QMQLVQSGPEVKKPGTSVKVSCKASGYTFTDYNVDWVRQARGQRLEWI |
| | IgG1 HC | GDINPNNGGTIYAQKFQERVTITVDKSTSTAYMELSSLRSEDTAVYYCAR |
| | | NYRWFGAMDHWGQGTTVTVSSASTKGPSVFPLAPSSKSTSGGTAALGCL |
| | | VKDYFPEPVTVSWNSGALTSGVHTFPAVLQSSGLYSLSSVVTVPSSSLGT |
| | | QTYICNVNHKPSNTKVDKKVEPKSCDKTHTCPPCPAPELLGGPSVFLFPPK |
| | | PKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPR |
| | | EPQVYTLPPSRDELTKNQVSLTCLVKGFYPSDIAVEWESNGQPENNYKTT |
| | | PPVLDSDGSFFLYSKLTVDKSRWQQGNVFSCSVMHEALHNHYTQKSLSL |
| | | SPGK |
| 54 | Anti-LAG3 Ab9, | QMQLVQSGPEVKKPGTSVKVSCKASGYTFTDYNVDWVRQARGQRLEWI |
| | IgG4-S228P HC | GDINPNGGGTIYAQKFQERVTITVDKSTSTAYMELSSLRSEDTAVYYCAR |
| | | NYRWFGAMDHWGQGTTVTVSSASTKGPSVFPLAPCSRSTSESTAALGCL |
| | | VKDYFPEPVTVSWNSGALTSGVHTFPAVLQSSGLYSLSSVVTVPSSSLGT |
| | | KTYTCNVDHKPSNTKVDKRVESKYGPPCPPCPAPEFLGGPSVFLFPPKPK |
| | | DTLMISRTPEVTCVVVDVSQEDPEVQFNWYVDGVEVHNAKTKPREEQFN |
| | | STYRVVSVLTVLHQDWLNGKEYKCKVSNKGLPSSIEKTISKAKGQPREPQ |
| | | VYTLPPSQEEMTKNQVSLTCLVKGFYPSDIAVEWESNGQPENNYKTTPPV |
| | | LDSDGSFFLYSRLTVDKSRWQEGNVFSCSVMHEALHNHYTQKSLSLSLG |
| | | K |
| 55 | Anti-LAG3 Ab8, | QMQLVQSGPEVKKPGTSVKVSCKASGYTFTDYNVDWVRQARGQRLEWI |
| | IgG4-S228P HC | GDINPNQGGTIYAQKFQERVTITVDKSTSTAYMELSSLRSEDTAVYYCAR |
| | | NYRWFGAMDHWGQGTTVTVSSASTKGPSVFPLAPCSRSTSESTAALGCL |
| | | VKDYFPEPVTVSWNSGALTSGVHTFPAVLQSSGLYSLSSVVTVPSSSLGT |
| | | KTYTCNVDHKPSNTKVDKRVESKYGPPCPPCPAPEFLGGPSVFLFPPKPK |
| | | DTLMISRTPEVTCVVVDVSQEDPEVQFNWYVDGVEVHNAKTKPREEQFN |
| | | STYRVVSVLTVLHQDWLNGKEYKCKVSNKGLPSSIEKTISKAKGQPREPQ |
| | | VYTLPPSQEEMTKNQVSLTCLVKGFYPSDIAVEWESNGQPENNYKTTPPV |
| | | LDSDGSFFLYSRLTVDKSRWQEGNVFSCSVMHEALHNHYTQKSLSLSLG |
| | | K |
| 56 | Anti-LAG3 Ab7, | QMQLVQSGPEVKKPGTSVKVSCKASGYTFTDYNVDWVRQARGQRLEWI |
| | IgG4-S228P HC | GDINPNSGGTIYAQKFQERVTITVDKSTSTAYMELSSLRSEDTAVYYCAR |
| | | NYRWFGAMDHWGQGTTVTVSSASTKGPSVFPLAPCSRSTSESTAALGCL |
| | | VKDYFPEPVTVSWNSGALTSGVHTFPAVLQSSGLYSLSSVVTVPSSSLGT |
| | | KTYTCNVDHKPSNTKVDKRVESKYGPPCPPCPAPEFLGGPSVFLFPPKPK |
| | | DTLMISRTPEVTCVVVDVSQEDPEVQFNWYVDGVEVHNAKTKPREEQFN |
| | | STYRVVSVLTVLHQDWLNGKEYKCKVSNKGLPSSIEKTISKAKGQPREPQ |
| | | VYTLPPSQEEMTKNQVSLTCLVKGFYPSDIAVEWESNGQPENNYKTTPPV |
| | | LDSDGSFFLYSRLTVDKSRWQEGNVFSCSVMHEALHNHYTQKSLSLSLG |
| | | K |
| 57 | Anti-LAG3 Ab5, | QMQLVQSGPEVKKPGTSVKVSCKASGYTFTDYNVDWVRQARGQRLEWI |
| | IgG4-S228P HC | GDINPNNGGTIYAQKFQERVTITVDKSTSTAYMELSSLRSEDTAVYYCAR |
| | | NYRWFGAMDHWGQGTTVTVSSASTKGPSVFPLAPCSRSTSESTAALGCL |
| | | VKDYFPEPVTVSWNSGALTSGVHTFPAVLQSSGLYSLSSVVTVPSSSLGT |
| | | KTYTCNVDHKPSNTKVDKRVESKYGPPCPPCPAPEFLGGPSVFLFPPKPK |
| | | DTLMISRTPEVTCVVVDVSQEDPEVQFNWYVDGVEVHNAKTKPREEQFN |
| | | STYRVVSVLTVLHQDWLNGKEYKCKVSNKGLPSSIEKTISKAKGQPREPQ |
| | | VYTLPPSQEEMTKNQVSLTCLVKGFYPSDIAVEWESNGQPENNYKTTPPV |
| | | LDSDGSFFLYSRLTVDKSRWQEGNVFSCSVMHEALHNHYTQKSLSLSLG |
| | | K |
| 58 | Anti-TIGIT | YIDPYNDGAKYNEKFKG |
| | antibody, VH- | |
| | CDR2 variant | |
| 59 | Anti-TIGIT | YIDPYNRGAKYNEKFG |
| | antibody, VH- | |
| | CDR2 variant | |
| 60 | Anti-TIGIT | YIDPYNLGAKYNEKFG |
| | antibody, VH- | |
| | CDR2 variant | |
| 61 | Anti-TIGIT | YIDPYNKGAKYNEKFG |
| | antibody, VH- | |
| | CDR2 variant | |
| 62 | Anti-TIGIT | YIDPYNFGAKYNEKFG |
| | antibody, VH- | |
| | CDR2 variant | |
| 63 | Anti-TIGIT | YIDPYNSGAKYNEKFG |
| | antibody, VH- | |
| | CDR2 variant | |
| 64 | Anti-TIGIT | YIDPYNYGAKYNEKFG |
| | antibody, VH- | |
| | CDR2 variant | |
| 65 | Anti-TIGIT | YIDPYNVGAKYNEKFG |
| | antibody, VH- | |
| | CDR2 variant | |
| 66 | Anti-TIGIT | YIDPYNDRAKYNEKFKG |
| | antibody, VH- | |
| | CDR2 variant | |
| 67 | Anti-TIGIT | YIDPYNDNAKYNEKFKG |
| | antibody, VH- | |
| | CDR2 variant | |
| 68 | Anti-TIGIT | YIDPYNDQAKYNEKFKG |
| | antibody, VH- | |
| | CDR2 variant | |
| 69 | Anti-TIGIT | YIDPYNDEAKYNEKFKG |
| | antibody, VH- | |
| | CDR2 variant | |
| 70 | Anti-TIGIT | YIDPYNDLAKYNEKEKG |
| | antibody, VH- | |
| | CDR2 variant | |
| 71 | Anti-TIGIT | YIDPYNDKAKYNEKFKG |
| | antibody, VH- | |
| | CDR2 variant | |
| 72 | Anti-TIGIT | YIDPYNDSAKYNEKFKG |
| | antibody, VH- | |
| | CDR2 variant | |
| 73 | Anti-TIGIT | YIDPYNDYAKYNEKFKG |
| | antibody, VH- | |
| | CDR2 variant | |
| 74 | Anti-TIGIT | YIDPYNDVAKYNEKFKG |
| | antibody, VH- | |
| | CDR2 variant | |
| 75 | Anti-TIGIT | YIDPYNDGAKYSQKFQG |
| | antibody, VH- | |
| | CDR2 variant | |
| 76 | Anti-TIGIT | YIDPYNX₇X₈AKYX₁₂X₁₃KFX₁₆G |
| | antibody, VH- | X₇ = D, R, L, K, F, S, Y or V |
| | CDR2 consensus | X₈ = G, R, N, Q, E, LK, S, Y or V |
| | | X₁₂ = N, A or S |
| | | X₁₃ = E or Q |
| | | X₁₆ = K or Q |
| 77 | Anti-TIGIT | GGPYGAYFDV |
| | antibody, VH- | |
| | CDR3 variant | |
| 78 | Anti-TIGIT | GGPYGDYFDV |
| | antibody, VH- | |
| | CDR3 variant | |
| 79 | Anti-TIGIT | GGPYGEYFDV |
| | antibody, VH- | |
| | CDR3 variant | |
| 80 | Anti-TIGIT | GGPYGFYFDV |
| | antibody, VH- | |
| | CDR3 variant | |
| 81 | Anti-TIGIT | GGPYGGYFDV |
| | antibody, VH- | |
| | CDR3 variant | |
| 82 | Anti-TIGIT | GGPYGIYFDV |
| | antibody, VH- | |
| | CDR3 variant | |
| 83 | Anti-TIGIT | GGPYGKYFDV |
| | antibody, VH- | |
| | CDR3 variant | |
| 84 | Anti-TIGIT | GGPYGNYFDV |
| | antibody, VH- | |
| | CDR3 variant | |
| 85 | Anti-TIGIT | GGPYGQYFDV |
| | antibody, VH- | |
| | CDR3 variant | |
| 86 | Anti-TIGIT | GGPYGRYFDV |
| | antibody, VH- | |
| | CDR3 variant | |
| 87 | Anti-TIGIT | GGPYGSYFDV |
| | antibody, VH- | |
| | CDR3 variant | |
| 88 | Anti-TIGIT | GGPYGTYFDV |
| | antibody, VH- | |
| | CDR3 variant | |
| 89 | Anti-TIGIT | GGPYGVYFDV |
| | antibody, VH- | |
| | CDR3 variant | |
| 90 | Anti-TIGIT | GGPYGYYFDV |
| | antibody, VH- | |
| | CDR3 variant | |
| 91 | Anti-TIGIT | GGPYGX₆YFDV |
| | antibody, VH- | X₆ = W, A, D, E, F, G, I, |
| | CDR3 consensus | K, N, Q, R, S, T, V or Y |
| 92 | Anti-TIGIT | AAKTLAE |
| | antibody, VL- | |
| | CDR2 variant | |
| 93 | Anti-TIGIT | YAKTLAE |
| | antibody, VL- | |
| | CDR2 variant | |
| 94 | Anti-TIGIT | WAKTLAE |
| | antibody, VL- | |
| | CDR2 variant | |
| 95 | Anti-TIGIT | SAKTLAE |
| | antibody, VL- | |
| | CDR2 variant | |
| 96 | Anti-TIGIT | TAKTLAE |
| | antibody, VL- | |
| | CDR2 variant | |
| 97 | Anti-TIGIT | JAKTLAE |
| | antibody, VL- | |
| | CDR2 variant | |
| 98 | Anti-TIGIT | VAKTLAE |
| | antibody, VL- | |
| | CDR2 variant | |
| 99 | Anti-TIGIT | NNKTLAE |
| | antibody, VL- | |
| | CDR2 variant | |
| 100 | Anti-TIGIT | NIKTLAE |
| | antibody, VL- | |
| | CDR2 variant | |
| 101 | Anti-TIGIT | NLLTLAE |
| | antibody, VL~ | |
| | CDR2 variant | |
| 102 | Anti-TIGIT | NTKTLAE |
| | antibody, VL- | |
| | CDR2 variant | |
| 103 | Anti-TIGIT | NVKTLAE |
| | antibody, VL- | |
| | CDR2 variant | |
| 104 | Anti-TIGIT | X₁X₂KTLAE |
| | antibody, VL- | X₁ = N, A, V, W, S, T, R, HG, I or V |
| | CDR2 consensus | X₂ = A, N, I, L, T or V |
| 105 | Anti-ILT4, VL- | TGSSSNIGAGYDVH |
| | CDR1 | |
| 106 | Anti-ILT4, VL- | GDSNRPS |
| | CDR2 | |
| 107 | Anti-ILT4, VL- | QSFDNSLSAYV |
| | CDR3 | |
| 108 | Anti-ILT4, VL | ESVLTQPPSVSGAPGQRVTISCTGSSSNIGAGYDVHWYQQLPGTAPKLLIY |
| | | GDSNRPSGVPDRFSVSKSGASASLAITGLQAEDEADYYCQSFDNSLSAYV |
| | | FGGGTQLTVL |
| 109 | Anti-ILT4, light | ESVLTQPPSVSGAPGQRVTISCTGSSSNIGAGYDVHWYQQLPGTAPKLLIY |
| | chain | GDSNRPSGVPDRESVSKSGASASLAITGLQAEDEADYYCQSFDNSLSAYV |
| | | FGGGTQLTVLGQPKAAPSVTLFPPSSEELQANKATLVCLISDFYPGAVTVA |
| | | WKADSSPVKAGVETTTPSKQSNNKYAASSYLSLTPEQWKSHRSYSCQVT |
| | | HEGSTVEKTVAPTECS |
| 110 | Anti-ILT4, VH- | GYYWS |
| | CDR1 | |
| 111 | Anti-ILT4, VH- | EINHAGSTNYNPSLKS |
| | CDR2 | |
| 112 | Anti-ILT4, VH- | LPTRWVTTRYFDL |
| | CDR3 | |
| 113 | Anti-ILT4, VH | EVQLQQWGAGLLKPSETLSLTCAVYGGSFSGYYWSWIRQPPGKGLEWIG |
| | | EINHAGSTNYNPSLKSRVTISVDTSKNQFSLKLSSVTAADTAVYYCARLPT |
| | | RWVTTRYFDLWGRGTLVTVSS |
| 114 | Anti-ILT4, heavy | EVQLQQWGAGLLKPSETLSLTCAVYGGSFSGYYWSWIRQPPGKGLEWIG |
| | chain | EINHAGSTNYNPSLKSRVTISVDTSKNQFSLKLSSVTAADTAVYYCARLPT |
| | | RWVTTRYFDLWGRGTLVTVSSASTKGPSVFPLAPCSRSTSESTAALGCLV |
| | | KDYFPEPVTVSWNSGALTSGVHTFPAVLQSSGLYSLSSVVTVPSSSLGTKT |
| | | YTCNVDHKPSNTKVDKRVESKYGPPCPPCPAPEFLGGPSVFLFPPKPKDTL |
| | | MISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDS |
| | | DGSFFLYSRLTVDKSRWQEGNVFSCSVMHEALHNHYTQKSLSLSLGK |

1. A method for determining a critical quality attribute of a co-formulation, the method comprising:
(i) preparing a sample of the co-formulation; and
(ii) performing an analytical method on the sample,
wherein the co-formulation comprises two or more different types of antibodies or antigen binding fragments thereof, and
wherein the analytical method measures the critical quality attribute of each of the two or more different types of antibodies or antigen binding fragments thereof simultaneously.

2. The method of claim 1, wherein:
(a) the co-formulation comprises an anti-PD-1 antibody or antigen binding fragment thereof and an anti-TIGIT antibody or antigen binding fragment thereof;
(b) the co-formulation comprises an anti-PD-1 antibody or antigen binding fragment thereof and an anti-LAG3 antibody or antigen binding fragment thereof: or
(c) the co-formulation comprises an anti-PD-1 antibody or antigen binding fragment thereof and an anti-ILT4 antibody or antigen binding fragment thereof.

3-8. (canceled)

9. The method of claim 2, wherein the co-formulation comprises an anti-PD-1 antibody or antigen binding fragment thereof and an anti-TIGIT antibody or antigen binding fragment thereof, and wherein the anti-PD-1 antibody or antigen binding fragment thereof comprises;
(a) a light chain variable region (V_{L}) complementarity determining region (CDR) 1, a V_{L} CDR2, and a V_{L} CDR3 comprising amino acid sequences as set forth in SEQ ID Nos:1, 2, and 3, respectively, and a heavy chain variable region (V_{H}) CDR1, a V_{H} CDR2, and a V_{H} CDR3 comprising amino acid sequences as set forth in SEQ ID NOS:6, 7, and 8, respectively;
(b) a V_{L} region comprising an amino acid sequence as set forth in SEO ID NO:4, and a V_{H} region comprising an amino acid sequence as set forth in SEO ID NO:9; or
(c) a light chain comprising or consisting of an amino acid sequence as set forth in SEO ID NO:5 and a heavy chain comprising or consisting of an amino acid sequence as set forth in SEO ID NO:10.

10-14. (canceled)

15. The method of claim 2, wherein the co-formulation comprises an anti-PD-1 antibody or antigen binding fragment thereof and an anti-TIGIT antibody or antigen binding fragment thereof, and wherein the anti-PD-1 antibody or antigen binding fragment thereof is pembrolizumab.

16-17. (canceled)

18. The method of claim 2, wherein the co-formulation comprises an anti-PD-1 antibody or antigen binding fragment thereof and an anti-TIGIT antibody or antigen binding fragment thereof, and wherein the anti-TIGIT antibody or antigen binding fragment thereof comprises
(a) a V_{L} CDR1, a V_{L} CDR2, and a V_{L} CDR3 comprising amino acid sequences as set forth in SEQ ID NOs:21, 22, and 23, respectively, and a V_{H} CDR1, a V_{H} CDR2, and a V_{H} CDR3 comprising amino acid sequences as set forth in SEQ ID NOs:26, 27, and 28, respectively;
(b) a V_{L} region comprising an amino acid sequence as set forth in SEO ID NO:24, and a V_{H} region comprising an amino acid sequence as set forth in SEO ID NO:29; or
(c) a light chain comprising or consisting of an amino acid sequence as set forth in SEO ID NO:25 and a heavy chain comprising or consisting of an amino acid sequence as set forth in SEO ID NO:30.

19-20. (canceled)

21. The method of claim 2, wherein the co-formulation comprises an anti-PD-1 antibody or antigen binding fragment thereof and an anti-TIGIT antibody or antigen binding fragment thereof, and wherein:
(i) (a) the anti-PD-1 antibody or antigen binding fragment thereof comprises a V_{L} CDR1, a V_{L} CDR2, and a V_{L} CDR3 comprising amino acid sequences as set forth in SEQ ID Nos:1, 2, and 3, respectively, and a V_{H} CDR1, a V_{H} CDR2, and a V_{H} CDR3 comprising amino acid sequences as set forth in SEQ ID Nos:6, 7, and 8, respectively; and
(b) the anti-TIGIT antibody or antigen binding fragment thereof comprises a V_{L} CDR1, a V_{L} CDR2, and a V_{L} CDR3 comprising amino acid sequences as set forth in SEQ ID Nos:21, 22, and 23, respectively, and a V_{H} CDR1, a V_{H} CDR2, and a V_{H} CDR3 comprising amino acid sequences as set forth in SEQ ID Nos:26, 27, and 28, respectively;
(ii) (a) the anti-PD-1 antibody or antigen binding fragment thereof comprises a V_{L} region comprising an amino acid sequence as set forth in SEO ID NO:4, and a V_{H} region comprising an amino acid sequence as set forth in SEO ID NO:9; and
(b) the anti-TIGIT antibody or antigen binding fragment thereof comprises a V_{L} region comprising an amino acid sequence as set forth in SEO ID NO:24, and a V_{H} region comprising an amino acid sequence as set forth in SEO ID NO:29: or
(iii) (a) the anti-PD-1 antibody or antigen binding fragment thereof comprises a light chain comprising or consisting of an amino acid sequence as set forth in SEO ID NO:5 and a heavy chain comprising or consisting of an amino acid sequence as set forth in SEO ID NO:10; and
(b) the anti-TIGIT antibody or antigen binding fragment thereof comprises a light chain comprising or consisting of an amino acid sequence as set forth in SEO ID NO:25 and a heavy chain comprising or consisting of an amino acid sequence as set forth in SEO ID NO:30.

22-23. (canceled)

24. The method of claim 2, wherein the co-formulation comprises an anti-PD-1 antibody or antigen binding fragment thereof and an anti-TIGIT antibody or antigen binding fragment thereof, and wherein a ratio of the anti-PD-1 antibody or antigen binding fragment thereof to the anti-TIGIT antibody or antigen binding fragment thereof is within a range of about 2:1 to 1:2.

25. The method of claim 1, wherein the analytical method excludes hydrophobic interaction chromatography (HIC) and reverse phase liquid chromatography (RPLC).

26. The method of claim 25, wherein the analytical method is a liquid chromatography-mass spectrometry (LC-MS) technique, optionally wherein the LC-MS technique comprises the use of a quadruple Dalton mass detector.

27. (canceled)

28. The method of claim 26, wherein the step of preparing the sample comprises digesting the two or more different types of antibodies by mixing a protease with the sample.

29. The method of claim 28, wherein the method comprises one or more features selected from:
(a) the protease is selected from the group consisting of Arg-C, Asp-N, chymotrypsin, elastase, endo H, Glu-C, IdeS Protease, IdeZ Protease, Lys-C, Lys-N, pepsin, PNGase F, rAsp-N, rLys-C, thermolysin, trypsin, and combinations thereof;
(b) the protease comprises Lys-C and a concentration of Lys-C in the sample is within a range of about 0.005 to 0.01 g/L; and
(c) the protease is mixed with the sample for about 60 mins to 70 mins at a temperature within a range of about 35° C. to 40° C.

30-32. (canceled)

33. The method of claim 28, wherein the step of digesting comprises mixing a reducing agent solution with the sample.

34. The method of claim 33, wherein;
the reducing agent solution comprises dithiothreitol and/or tris(2-carboxyethyl)phosphine; and/or
the reducing agent solution is mixed with the sample for about 25 mins to 35 mins at a temperature within a range of about 35° C. to 40° C.

35. (canceled)

36. The method of claim 28, wherein the step of digesting comprises mixing an alkylating agent with the sample.

37. The method of claim 36, wherein:
the alkylating agent comprises iodoacetamide; and/or
the alkylating agent is mixed with the sample for about 25 mins to 35 mins at a temperature within a range of about 35° C. to 40° C.

38. (canceled)

39. The method of claim 26, wherein the analytical method comprises (i) applying the co-formulation to a chromatography material; and (ii) eluting with a solution comprising a mobile phase A and a mobile phase B.

40. The method of claim 39, wherein the method comprises one or more features selected from:
(a) the mobile phase A comprises formic acid or trifluoracetic acid;
(b) the mobile phase B comprises formic acid or trifluoracetic acid;
(c) the mobile phase A comprises acetic acid in water;
(d) the mobile phase B comprises acetic acid in acetonitrile;
(e) an initial ratio of the mobile phase B to the mobile phase A is within a range of about 10% to 20% with a flow rate within a range of about 0.1 mL/min to 1 mL/min;
(f) the chromatography is conducted at a temperature within a range of a range of about 60° C. to 100° C.;
(g) the chromatography is ultra-performance liquid chromatography (UPLC); and
(h) the elution is a gradient elution.

41-47. (canceled)

48. The method of claim 26, wherein the LC-MS technique comprises the use of a quadruple Dalton mass detector, and wherein an electrospray ionization probe of the quadruple Dalton mass detector is at a temperature within a range of about 350° C. to 450° C.; and/or a capillary voltage of the quadruple Dalton mass detector is at a temperature within a range of about 1 to 2 kV.

49-142. (canceled)

143. The method of claim 1, wherein the critical quality attribute is selected from the group consisting of oxidation, isomerization, deamidation, disulfide bond modification, and glycosylation.

144-146. (canceled)

147. A method for determining a critical quality attribute of a co-formulation comprising an anti-PD-1 antibody and an anti-TIGIT antibody, the method comprising:
(i) preparing a sample of the co-formulation; and
(ii) performing a liquid chromatography-mass spectrometry (LC-MS) technique on the sample, wherein the LC-MS measures the critical quality attribute,
optionally wherein the critical quality attribute of the anti-PD-1 antibody is an oxidation at an amino acid corresponding to M105 in SEO ID NO: 8, 9 or 10 and/or the critical quality attribute of the anti-TIGIT antibody is an oxidation at an amino acid corresponding to W104 in SEO ID NO: 28, 29 or 30.

148-153. (canceled)

154. A method for determining a critical quality attribute of a co-formulation comprising an anti-PD-1 antibody and an anti-LAG3 antibody, the method comprising:
(i) preparing a sample of the co-formulation; and
(ii) performing a liquid chromatography-mass spectrometry (LC-MS) technique on the sample, wherein the LC-MS measures the critical quality attribute,
optionally wherein the critical quality attribute of the anti-PD-1 antibody is an oxidation at an amino acid corresponding to M105 in SEO ID NO: 8, 9 or 10 and/or the critical quality attribute of the anti-LAG3 antibody is an oxidation at an amino acid corresponding to W102 in SEO ID NO: 18, 19 or 20.

155-160. (canceled)

161. A method for determining a critical quality attribute of a co-formulation comprising an anti-PD-1 antibody and an anti-ILT4 antibody, the method comprising:
(i) preparing a sample of the co-formulation; and
(ii) performing a liquid chromatography-mass spectrometry (LC-MS) technique on the sample, wherein the LC-MS measures the critical quality attribute,
optionally wherein the critical quality attribute of the anti-PD-1 antibody is an oxidation at an amino acid corresponding to M105 in SEO ID NO: 8, 9, or 10 and/or the critical quality attribute of the anti-ILT4 antibody is an oxidation at an amino acid corresponding to W102 in SEO ID NO: 112, 113 or 114.

162-181. (canceled)


SEQUENCE LISTING (116 sequences)

SEQ ID NO: 1 - protein, 15 aa, synthetic construct
RASKGVSTSGYSYLH

SEQ ID NO: 2 - protein, 7 aa, synthetic construct
LASYLES

SEQ ID NO: 3 - protein, 9 aa, synthetic construct
QHSRDLPLT

SEQ ID NO: 4 - protein, 111 aa, synthetic construct [sequence omitted]

SEQ ID NO: 5 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, synthetic construct
NYYMY

SEQ ID NO: 7 - protein, 17 aa, synthetic construct
GINPSNGGTNFNEKFKN

SEQ ID NO: 8 - protein, 11 aa, synthetic construct
RDYRFDMGFDY

SEQ ID NO: 9 - protein, 120 aa, synthetic construct [sequence omitted]

SEQ ID NO: 10 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 15 aa, synthetic construct
KASQSLDYEGDSDMN

SEQ ID NO: 12 - protein, 7 aa, synthetic construct
GASNLES

SEQ ID NO: 13 - protein, 9 aa, synthetic construct
QQSTEDPRT

SEQ ID NO: 14 - protein, 111 aa, synthetic construct
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIK

SEQ ID NO: 15 - protein, 218 aa, synthetic construct
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, synthetic construct
DYNVD

SEQ ID NO: 17 - protein, 17 aa, synthetic construct
DINPNDGGTIYAQKFQE

SEQ ID NO: 18 - protein, 10 aa, synthetic construct
NYRWFGAMDH

SEQ ID NO: 19 - protein, 119 aa, synthetic construct
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSS

SEQ ID NO: 20 - protein, 446 aa, synthetic construct
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 11 aa, synthetic construct
RASEHIYSYLS

SEQ ID NO: 22 - protein, 7 aa, synthetic construct
NAKTLAE

SEQ ID NO: 23 - protein, 9 aa, synthetic construct
QHHFGSPLT

SEQ ID NO: 24 - protein, 107 aa, synthetic construct
DIQMTQSPSSLSASVGDRVTITCRASEHIYSYLSWYQQKPGKVPKLLIYNAKTLAEGVPSRFSGSGSGTDFTLTISSLQPEDVATYYCQHHFGSPLTFGQGTRLEIK

SEQ ID NO: 25 - protein, 214 aa, synthetic construct
DIQMTQSPSSLSASVGDRVTITCRASEHIYSYLSWYQQKPGKVPKLLIYNAKTLAEGVPSRFSGSGSGTDFTLTISSLQPEDVATYYCQHHFGSPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 5 aa, synthetic construct
SYVMH

SEQ ID NO: 27 - protein, 17 aa, synthetic construct
YIDPYNDGAKYAQKFQG

SEQ ID NO: 28 - protein, 10 aa, synthetic construct
GGPYGWYFDV

SEQ ID NO: 29 - protein, 119 aa, synthetic construct
EVQLVQSGAEVKKPGSSVKVSCKASGYTFSSYVMHWVRQAPGQGLEWIGYIDPYNDGAKYAQKFQGRVTLTSDKSTSTAYMELSSLRSEDTAVYYCARGGPYGWYFDVWGQGTTVTVSS

SEQ ID NO: 30 - protein, 449 aa, synthetic construct
EVQLVQSGAEVKKPGSSVKVSCKASGYTFSSYVMHWVRQAPGQGLEWIGYIDPYNDGAKYAQKFQGRVTLTSDKSTSTAYMELSSLRSEDTAVYYCARGGPYGWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 11 aa, synthetic construct
RASQSVSSYLA

SEQ ID NO: 32 - protein, 7 aa, synthetic construct
DASNRAT

SEQ ID NO: 33 - protein, 9 aa, synthetic construct
QQSSNWPRT

SEQ ID NO: 34 - protein, 107 aa, synthetic construct [sequence omitted]

SEQ ID NO: 35 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 5 aa, synthetic construct
NSGMH

SEQ ID NO: 37 - protein, 17 aa, synthetic construct
VIWYDGSKRYYADSVKG

SEQ ID NO: 38 - protein, 4 aa, synthetic construct
NDDY

SEQ ID NO: 39 - protein, 113 aa, synthetic construct [sequence omitted]

SEQ ID NO: 40 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 17 aa, synthetic construct
DINPNGGGTIYAQKFQE

SEQ ID NO: 42 - protein, 17 aa, synthetic construct
DINPNQGGTIYAQKFQE

SEQ ID NO: 43 - protein, 17 aa, synthetic construct
DINPNSGGTIYAQKFQE

SEQ ID NO: 44 - protein, 17 aa, synthetic construct
DINPNNGGTIYAQKFQE

SEQ ID NO: 45 - protein, 119 aa, synthetic construct
QMQLVQSGPEVKKPGTSVKVSCKASGYTFTDYNVDWVRQARGQRLEWIGDINPNGGGTIYAQKFQERVTITVDKSTSTAYMELSSLRSEDTAVYYCARNYRWFGAMDHWGQGTTVTVSS

SEQ ID NO: 46 - protein, 119 aa, synthetic construct
QMQLVQSGPEVKKPGTSVKVSCKASGYTFTDYNVDWVRQARGQRLEWIGDINPNQGGTIYAQKFQERVTITVDKSTSTAYMELSSLRSEDTAVYYCARNYRWFGAMDHWGQGTTVTVSS

SEQ ID NO: 47 - protein, 119 aa, synthetic construct
QMQLVQSGPEVKKPGTSVKVSCKASGYTFTDYNVDWVRQARGQRLEWIGDINPNSGGTIYAQKFQERVTITVDKSTSTAYMELSSLRSEDTAVYYCARNYRWFGAMDHWGQGTTVTVSS

SEQ ID NO: 48 - protein, 119 aa, synthetic construct
QMQLVQSGPEVKKPGTSVKVSCKASGYTFTDYNVDWVRQARGQRLEWIGDINPNNGGTIYAQKFQERVTITVDKSTSTAYMELSSLRSEDTAVYYCARNYRWFGAMDHWGQGTTVTVSS

SEQ ID NO: 49 - protein, 449 aa, synthetic construct
QMQLVQSGPEVKKPGTSVKVSCKASGYTFTDYNVDWVRQARGQRLEWIGDINPNGGGTIYAQKFQERVTITVDKSTSTAYMELSSLRSEDTAVYYCARNYRWFGAMDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 449 aa, synthetic construct
QMQLVQSGPEVKKPGTSVKVSCKASGYTFTDYNVDWVRQARGQRLEWIGDINPNQGGTIYAQKFQERVTITVDKSTSTAYMELSSLRSEDTAVYYCARNYRWFGAMDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 449 aa, synthetic construct
QMQLVQSGPEVKKPGTSVKVSCKASGYTFTDYNVDWVRQARGQRLEWIGDINPNSGGTIYAQKFQERVTITVDKSTSTAYMELSSLRSEDTAVYYCARNYRWFGAMDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 449 aa, synthetic construct
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 449 aa, synthetic construct
QMQLVQSGPEVKKPGTSVKVSCKASGYTFTDYNVDWVRQARGQRLEWIGDINPNNGGTIYAQKFQERVTITVDKSTSTAYMELSSLRSEDTAVYYCARNYRWFGAMDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 446 aa, synthetic construct
QMQLVQSGPEVKKPGTSVKVSCKASGYTFTDYNVDWVRQARGQRLEWIGDINPNGGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - protein, 446 aa, synthetic construct
QMQLVQSGPEVKKPGTSVKVSCKASGYTFTDYNVDWVRQARGQRLEWIGDINPNQGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 56 - protein, 446 aa, synthetic construct
QMQLVQSGPEVKKPGTSVKVSCKASGYTFTDYNVDWVRQARGQRLEWIGDINPNSGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 446 aa, synthetic construct
QMQLVQSGPEVKKPGTSVKVSCKASGYTFTDYNVDWVRQARGQRLEWIGDINPNNGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 58 - protein, 17 aa, synthetic construct
YIDPYNDGAKYNEKFKG

SEQ ID NO: 59 - protein, 16 aa, synthetic construct
YIDPYNRGAKYNEKFG

SEQ ID NO: 60 - protein, 16 aa, synthetic construct
YIDPYNLGAKYNEKFG

SEQ ID NO: 61 - protein, 16 aa, synthetic construct
YIDPYNKGAKYNEKFG

SEQ ID NO: 62 - protein, 16 aa, synthetic construct
YIDPYNFGAKYNEKFG

SEQ ID NO: 63 - protein, 16 aa, synthetic construct
YIDPYNSGAKYNEKFG

SEQ ID NO: 64 - protein, 16 aa, synthetic construct
YIDPYNYGAKYNEKFG

SEQ ID NO: 65 - protein, 16 aa, synthetic construct
YIDPYNVGAKYNEKFG

SEQ ID NO: 66 - protein, 17 aa, synthetic construct
YIDPYNDRAKYNEKFKG

SEQ ID NO: 67 - protein, 17 aa, synthetic construct
YIDPYNDNAKYNEKFKG

SEQ ID NO: 68 - protein, 17 aa, synthetic construct
YIDPYNDQAKYNEKFKG

SEQ ID NO: 69 - protein, 17 aa, synthetic construct
YIDPYNDEAKYNEKFKG

SEQ ID NO: 70 - protein, 17 aa, synthetic construct
YIDPYNDLAKYNEKFKG

SEQ ID NO: 71 - protein, 17 aa, synthetic construct
YIDPYNDKAKYNEKFKG

SEQ ID NO: 72 - protein, 17 aa, synthetic construct
YIDPYNDSAKYNEKFKG

SEQ ID NO: 73 - protein, 17 aa, synthetic construct
YIDPYNDYAKYNEKFKG

SEQ ID NO: 74 - protein, 17 aa, synthetic construct
YIDPYNDVAKYNEKFKG

SEQ ID NO: 75 - protein, 17 aa, synthetic construct
YIDPYNDGAKYSQKFQG

SEQ ID NO: 76 - protein, 17 aa, synthetic construct
  VARIANT 7: note = D, R, L, K, F, S, Y or V
  VARIANT 8: note = G, R, N, Q, E, L, K, S, Y or V
  VARIANT 12: note = N, A or S
  VARIANT 13: note = E or Q
  VARIANT 16: note = K or Q
YIDPYNXXAKYXXKFXG

SEQ ID NO: 77 - protein, 10 aa, synthetic construct
GGPYGAYFDV

SEQ ID NO: 78 - protein, 10 aa, synthetic construct
GGPYGDYFDV

SEQ ID NO: 79 - protein, 10 aa, synthetic construct
GGPYGEYFDV

SEQ ID NO: 80 - protein, 10 aa, synthetic construct
GGPYGFYFDV

SEQ ID NO: 81 - protein, 10 aa, synthetic construct
GGPYGGYFDV

SEQ ID NO: 82 - protein, 10 aa, synthetic construct
GGPYGIYFDV

SEQ ID NO: 83 - protein, 10 aa, synthetic construct
GGPYGKYFDV

SEQ ID NO: 84 - protein, 10 aa, synthetic construct
GGPYGNYFDV

SEQ ID NO: 85 - protein, 10 aa, synthetic construct
GGPYGQYFDV

SEQ ID NO: 86 - protein, 10 aa, synthetic construct
GGPYGRYFDV

SEQ ID NO: 87 - protein, 10 aa, synthetic construct
GGPYGSYFDV

SEQ ID NO: 88 - protein, 10 aa, synthetic construct
GGPYGTYFDV

SEQ ID NO: 89 - protein, 10 aa, synthetic construct
GGPYGVYFDV

SEQ ID NO: 90 - protein, 10 aa, synthetic construct
GGPYGYYFDV

SEQ ID NO: 91 - protein, 10 aa, synthetic construct
  VARIANT 6: note = W, A, D, E, F, G, I, K, N, Q, R, S, T, V or Y
GGPYGXYFDV

SEQ ID NO: 92 - protein, 7 aa, synthetic construct
AAKTLAE

SEQ ID NO: 93 - protein, 7 aa, synthetic construct
YAKTLAE

SEQ ID NO: 94 - protein, 7 aa, synthetic construct
WAKTLAE

SEQ ID NO: 95 - protein, 7 aa, synthetic construct
SAKTLAE

SEQ ID NO: 96 - protein, 7 aa, synthetic construct
TAKTLAE

SEQ ID NO: 97 - protein, 7 aa, synthetic construct
IAKTLAE

SEQ ID NO: 98 - protein, 7 aa, synthetic construct
VAKTLAE

SEQ ID NO: 99 - protein, 7 aa, synthetic construct
NNKTLAE

SEQ ID NO: 100 - protein, 7 aa, synthetic construct
NIKTLAE

SEQ ID NO: 101 - protein, 7 aa, synthetic construct
NLLTLAE

SEQ ID NO: 102 - protein, 7 aa, synthetic construct
NTKTLAE

SEQ ID NO: 103 - protein, 7 aa, synthetic construct
NVKTLAE

SEQ ID NO: 104 - protein, 7 aa, synthetic construct
  VARIANT 1: note = N, A, V, W, S, T, R, H, G, I or V
  VARIANT 2: note = A, N, I, L, T or V
XXKTLAE

SEQ ID NO: 105 - protein, 14 aa, synthetic construct
TGSSSNIGAGYDVH

SEQ ID NO: 106 - protein, 7 aa, synthetic construct
GDSNRPS

SEQ ID NO: 107 - protein, 11 aa, synthetic construct
QSFDNSLSAYV

SEQ ID NO: 108 - protein, 111 aa, synthetic construct
ESVLTQPPSVSGAPGQRVTISCTGSSSNIGAGYDVHWYQQLPGTAPKLLIYGDSNRPSGVPDRFSVSKSGASASLAITGLQAEDEADYYCQSFDNSLSAYVFGGGTQLTVL

SEQ ID NO: 109 - protein, 217 aa, synthetic construct
ESVLTQPPSVSGAPGQRVTISCTGSSSNIGAGYDVHWYQQLPGTAPKLLIYGDSNRPSGVPDRFSVSKSGASASLAITGLQAEDEADYYCQSFDNSLSAYVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 110 - protein, 5 aa, synthetic construct
GYYWS

SEQ ID NO: 111 - protein, 16 aa, synthetic construct
EINHAGSTNYNPSLKS

SEQ ID NO: 112 - protein, 13 aa, synthetic construct
LPTRWVTTRYFDL

SEQ ID NO: 113 - protein, 121 aa, synthetic construct
EVQLQQWGAGLLKPSETLSLTCAVYGGSFSGYYWSWIRQPPGKGLEWIGEINHAGSTNYNPSLKSRVTISVDTSKNQFSLKLSSVTAADTAVYYCARLPTRWVTTRYFDLWGRGTLVTVSS

SEQ ID NO: 114 - protein, 448 aa, synthetic construct
EVQLQQWGAGLLKPSETLSLTCAVYGGSFSGYYWSWIRQPPGKGLEWIGEINHAGSTNYNPSLKSRVTISVDTSKNQFSLKLSSVTAADTAVYYCARLPTRWVTTRYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 115 - protein, 50 aa, synthetic construct
SGLNLMFEIKLIKWSNFLGEFNWLGILSKESLGIKWLFENNGLSWEFLIK

SEQ ID NO: 116 - protein, 50 aa, synthetic construct
KIFNEWGLLSELSWNIFKGLWENKLGWFLILSKEIFGNLWFNILGKLEWS